(19) United States
(12) Patent Application Publication
(10) Pub. No.: US 2021/0315915 A1
(43) Pub. Date: Oct. 14, 2021

(54) PHARMACEUTICAL COMPOSITIONS OF RIBAVIRIN

(71) Applicants: GlaxoSmithKline Intellectual Property (No.2) Limited, Brentford, Middlesex (GB); Liquidia Technologies, Inc., Morrisville, NC (US)

(72) Inventors: Philip Charles DELL'ORCO, King of Prussia, PA (US); Brian T FARRER, Morrisville, NC (US); Zhi HONG, Durham, NC (US); Christopher D ROBERTS, Belmont, MA (US); John Robert SAVAGE, Morrisville, NC (US); Jacob J SPRAGUE, Morrisville, NC (US); Yongcai WANG, Morrisville, NC (US)

(21) Appl. No.: 15/776,140

(22) PCT Filed: Nov. 18, 2016
(86) PCT No.: PCT/IB2016/056981

§ 371 Date: May 15, 2018

Related U.S. Application Data
(60) Provisional application No. 62/256,868, filed on Nov. 18, 2015.

(51) Int. Cl. A61K 31/7056; A61K 9/00; A61K 9/16

(52) U.S. Cl. CPC A61K 31/7056; A61K 9/1688; A61K 9/0075

(57) ABSTRACT

Disclosed are inhalable antiviral pharmaceutical compositions of ribavirin, methods of producing the same, and the use of such compositions in the treatment of virally associated respiratory infections, and associated diseases and conditions.

### FIELD OF THE INVENTION

The present invention relates to non-aqueous, inhalable pharmaceutical compositions of ribavirin, methods of producing the same, and the use of such compositions in the treatment of virally associated respiratory infections, and associated diseases and conditions.

### BACKGROUND OF THE INVENTION

Acute exacerbations of chronic obstructive pulmonary disease (COPD), asthma, and cystic fibrosis are major causes of morbidity and mortality. Kurai, et al., “Virus-Induced exacerbations in asthma and COPD”, Front. Microbiol., 4: 293 (2013). A growing body of clinical observations are consistent with a causal relationship between viral infection (human rhinovirus (HRV)/influenza virus/respiratory syncytial virus (RSV)/human metapneumovirus (hMPV)/adenovirus/parainfluenza virus (PIV)) and a significant percentage of exacerbations (i.e., 20-60%). See id. Additionally, virally induced airway damage is increasingly thought to prime secondary bacterial infections, which themselves account for 25-40% of exacerbations. Typically, the disease course of viral induced exacerbations is an initial upper respiratory tract (URT) infection followed by progression to the lower respiratory tract (LRT) over 4-6 days. Viral replication in the LRT is then sustained in COPD patients relative to otherwise healthy individuals (i.e., up to 21 days relative to <5 days).

These observations suggest that the preemptive administration of an antiviral agent to the LRT at the onset of URT symptoms could be a sound intervention strategy. The ideal antiviral agent would have broad spectrum activity to address the diversity of viral pathogens capable of inducing exacerbations. It is likely that intervention as early as possible will improve outcomes. Thus, there is an ongoing need for an inhaled antiviral to be available to COPD patients with a frequent exacerbation phenotype, to provide a rapid start of therapy upon URT symptoms.

Ribavirin is a marketed broad-spectrum antiviral compound. In the United States, ribavirin containing dosage forms include oral tablets (e.g., sold under the COPEGUS® brand name), oral capsules (e.g., REBETOL®, RIBASPHE®), oral solutions (FDA tentative approval), as an injectable solution (in combination with Pegainterferin Alfa-2 under the brand name PEGINTERFERON®, and PEGINTRON/REBETOL COMBO PACK®), as well as in the form of an inhaled solution (VIRAZOLE®). The approved therapeutic indications for the oral capsule, tablet, solution and injectable form products collectively include treatment of Chronic Hepatitis C (CHC) when prescribed in combination with (pegylated and nonpegylated) interferon alfa-2b, and the treatment of Chronic Hepatitis B (CHB). In the nebulized inhaled form, ribavirin has been approved for the treatment of severe respiratory syncytial virus (RSV) infections.

One drawback to ribavirin is that some patients taking ribavirin may experience significant drops in red blood cell count, and develop associated anemia. Further, ribavirin may also produce testicular lesions in rodents, and is considered teratogenic and/or produces embryocidal effects in certain animal species which have been studied.

The FDA approved labeling, for example, with Rebotol® (ribavirin in combination with Peglntron™), indicates that ribavirin has a multiple-dose half-life of 12-days, and it may persist in nonplasma compartments for as long as 6 months. Because of this, Rebetol® therapy is contraindicated in women who are pregnant and in the male partners of women who are pregnant. Great care must be taken to avoid pregnancy during therapy and for 6 months after completion of treatment in both female patients and in female partners of male patients who are taking Rebetol® therapy.

As an inhaled formulation, ribavirin is available in the United States as a nebulized solution, and is sold under the brand name Virazole®. Virazole® is indicated for the treatment of hospitalized infants and young children with severe lower respiratory tract infections due to RSV. The indicated administration for Virazole® takes place over a very long period of time and under very specific conditions. Virazole® is administered as an aqueous solution and is given in a hospital setting with a special nebulizer attached to an oxygen tent, a face mask, or a ventilator. According to label dosage and administration instructions, the recommended treatment regimen is 20 mg/mL Virazole® as the starting solution in the drug reservoir of the Valeant Small Particle Aerosol Generator SPAG-2 unit, with continuous aerosol administration for 12-18 hours per day for 3 to 7 days. With the recommended drug concentration of 20 mg/mL, the average aerosol concentration for a 12 hour delivery period would be 190 micrograms/liter of air.

The administration of Virazole® in non-mechanically ventilated infants, involves delivering Virazole® to an infant oxygen hood from the SPAG-2 aerosol generator. Administration by face mask or oxygen tent may be necessary if a hood cannot be employed. Further, because the volume and condensation area are larger in a tent, the setting may alter delivery dynamics of the drug.

The recommended dose and administration schedule for infants who require mechanical ventilation is the same as for those who do not. Either a pressure or volume cycle ventilator may be used in conjunction with the SPAG-2. In either case, it is recommended that patients should have their endotracheal tubes suctioned every 1-2 hours, and their pulmonary pressures monitored frequently (every 2-4 hours). For both pressure and volume ventilators, heated wire connective tubing and bacteria filters in series in the expiratory limb of the system (which must be changed frequently, i.e., every 4 hours) must be used to minimize the risk of Virazole® precipitation in the system and the subsequent risk of ventilator dysfunction.

As can be appreciated, the aerosolization of ribavirin in a tent or as a facemask may pose risks to anyone in proximity to the patient being treated, including medical staff and family members of the patient, who may breathe or otherwise be exposed to ribavirin in the air and many of whom will be of child bearing age. Thus, a major limitation of ribavirin use in RSV infected infants is the labeled indication for nebulized administration that results in high doses (6 grams/day), long (12-18 hours) dose administration, and high patient-to-patient variability. In addition, aerosolized ribavirin has been reported to cause moderate and long term bronchospasms, aggravating the clinical evolution of the disease. See Ventura, F., et al., “Is the use of ribavirin aerosols in respiratory syncytial virus infections justified? Clinical and economic evaluation” Arch Pediatr. February; 5(2):123-31 (1998). The prescribing information for Virazole® warns of pulmonary deterioration in COPD and asthma patients, and minor abnormalities in pulmonary function in healthy volunteers. Additional data is provided in the Virazole® NDA. Thus treatment can cause bronchospasm in COPD patients, which is believed to be linked to the mass of hypotonic particles being inhaled from an aqueous formulation. See, Walsh, B. et al., “Characterization of Ribavirin Aerosol With Small Particle Aerosol Generator and Vibrating Mesh Micropump Aerosol Technologies” Respir Care 2016, 61, 577-585.

Recent advances in inhaled drug delivery technology provide an opportunity to improve the efficiency of ribavirin administration and exploit its broad spectrum antiviral activity. Alternatives to aqueous nebulized formulations include metered dose inhalers, which deliver active pharmaceutical ingredients as a solution or suspension via a pressurized liquid propellant; and dry powder formulations, such as milled sized-reduced active pharmaceutical particles, spray dried inhalable dry powders, etc.

Such inhaled preparations have been discussed in the art. For example, descriptions of inhaled formulations, which are alternatives to nebulized solutions, include WO 2009/095681 and WO 2009/143011. Despite such disclosures, ribavirin has not been marketed in the United States in such alternative forms. Thus, there is a need to improve upon the formulation options available.

It is a goal of the present invention to provide an inhalable form of ribavirin which addresses one or more of these such shortcomings, and which allows the efficient delivery of ribavirin to and throughout the pulmonary system, with sufficient drug loading in a formulation to permit reduced drug delivery times when compared to a nebulized formulation, while reducing the release of the compound into the general environment, thus reducing the risk of exposure to those in proximity to the patient being treated with the compound, and avoid adverse reactions, e.g., bronchospasm.

### SUMMARY OF THE INVENTION

The present invention, in one aspect, provides a pharmaceutical composition comprising fabricated particles comprising ribavirin and optionally one or more excipients, which are described herein. In certain embodiments, the fabricated particles have a mass median aerodynamic diameter (MMAD) ranging from about 0.5 μm to about 6 μm. In certain aspects, the ribavirin is in a substantially crystalline form.

In a further aspect of the invention, the fabricated particles are formed by molding the ribavirin and optional excipient in mold cavities. In some embodiments, after the molding process, the fabricated particles have a substantially uniform shape, and are non-spherical. For example, the fabricated particles may comprise two substantially parallel surfaces, with in some instances, such parallel surfaces having substantially equal linear dimensions. The bulk density of the pharmaceutical compositions of the present invention are generally less than about 3.0 g/cm³, for example less than 2.5 g/cm³, less than 2.0 g/cm³, less than 1.5 g/cm³, and less than 1.0 g/cm³.

A further aspect of the invention provides fabricated particles wherein the excipient comprises a carbohydrate, an amino acid, a polypeptide, a synthetic polymer, a natural polymer, or mixtures thereof.

A further aspect of the invention provides fabricated particles where each particle has a substantially similar volume and substantially similar three-dimensional shape, which, in part, contribute to the percent or quantity of particles that reach the lung. In some embodiments in rat models, in vivo lung Cmax values are at least 5 times higher, and greater than 10 times higher, than micronized drug with the same drug dose. In other embodiments, in vivo lung Cmax values are at least 10, 15, 20, or 25 times higher than particles made through micronization with the same drug dose.

The respiratory tract may be thought of as containing nose, mouth, thoracic, bronchiolar, and alveolar compartments. Each compartment had two sub-compartments, the epithelial lining fluid (mucus) and the epithelial cells. Dissolved ribavirin concentration levels in the bronchiolar epithelial lining fluid (BELF) may be an useful indication of a ribavirin formulation ability achieving a Cmax capable of preventing or reducing viral replication in the epithelial cells thus reduce the frequency and severity of acute exacerbations triggered by viruses such as Respiratory Syncytial virus (RSV), human rhinovirus (HRV), MERS, SARS, influenza virus, parainfluenza, human metapneumovirus, adenovirus, coronavirus, and/or picornavirus.

Sampling of BELF allows dissolved RBV Cmax to be determined. The dissolved RBV present in bronchiolar epithelial lining fluid may be determined from analysis of a retrieved BELF lavage. For example, lavage may be performed within 60 minutes after delivery of the inhaled composition by inhalation, under sedation or local anesthesia, using saline which is flushed and retrieved (e.g. 4×50 mL washes) from the bronchiolar compartment. Cmax of dissolved RBV in the BELF prior to lavage may be determined/derived from the measured total ribavirin quantity in the retrieved lavage fluid (whose RBV total contains both the previously dissolved and undissolved BELF amounts) by physiologically based pharmacokinetic modelling (PBPK).

Thus, a still further aspect of the invention relates to a powder ribavirin composition which achieves a determined Cmax of dissolved ribavirin in bronchial epithelial lung fluid from 10 μM to 10mM, such as in on embodiment 100 μM to 1mM, in another 10 μM to 1mM, in a further 10 μM to 500 μM, in a still further 50 μM to 500 μM, and still further 50 μM to 100 μM.

In some embodiments, the shape factor of the particles includes a solid wafer shaped “pollen” particle with two substantially planar, substantially parallel surfaces comprising a substantial majority of the surface area of the particle. In some embodiments, the particle shape includes a cylindrical solid volume.

The invention also relates to dose containment of the fabricated particles, and to inhaler devices for delivery of the pharmaceutical composition.

These and other objects and features of the invention will become more fully apparent when the following detailed description is read in conjunction with the accompanying figures and examples,

Abbreviations:

ΔHm: melting enthalpy

μM: microMolar

mM: milliMolar

Amb: ambient

DSC: differential scanning calorimetry

PC: polycarbonate

PES: polyethersulfone

PTFE: polytetrafluoroethylene

PVDF: polyvinylidene fluoride

PVOH: polyvinyl alcohol

RBV: ribavirin

RH: relative humidity

Tm: melting temperature

WFI: water for injection

XRPD: X-Ray Powder Diffraction

### BRIEF DESCRIPTION OF THE DRAWINGS

FIGS. 1A, 1B, 2A and 2B are scanning electron microscopy (SEM) images of “pollen” shaped particles.

FIG. 3A is an SEM image of cylindrically-shaped particles.

FIG. 3B is a line drawing of a cylindrical particle.

FIG. 4 is a diagram showing a fabrication process according to the present invention.

FIG. 5 is a further process diagram relating to production of crystalline particles.

FIG. 6 is next generation impactor (NGI) characterization of ribavirin fabricated particles.

FIG. 7 is a shows comparative ribavirin concentration measurement in rat lung homogenates following a single inhaled dose of representative ribavirin containing fabricated particles versus a micronized lactose formulation.

FIG. 8 shows composite mean plasma and lung concentrations of crystalline ribavirin.

FIG. 9 shows composite mean plasma and lung concentrations of ribavirin with lactose.

FIG. 10 depicts the mass median aerodynamic diameter (MMAD) of stored crystalline ribavirin PRINT® particles.

FIG. 11 depicts the fine particle fraction of stored crystalline ribavirin PRINT® particles.

FIG. 12 depicts the deposition summary for Ribavirin:Trehalose:Trileucine PRINT® particles.

FIG. 13 depicts the deposition summary for Dextran:Ribavirin:PVOH PRINT® particles.

FIG. 14 depicts the deposition pattern for crystalline ribavirin PRINT® particles at 6 months.

FIG. 15 depicts the deposition pattern for crystalline ribavirin PRINT® particles at 2 months.

FIG. 16A depicts XRPD data for crystalline ribavirin PRINT® particles.

FIG. 16B depicts DSC data for crystalline ribavirin PRINT® particles stored under various conditions for 1 month.

FIG. 17 depicts the ribavirin dissolution profiles for various ribavirin materials.

FIG. 18 is an XRPD plot for RBV polymorphic Form I.

FIG. 19 is an XRPD plot for RBV polymorphic Form II.

FIG. 20 shows the XRPD patterns for 99:1 w:w RBV:PVOH material tested at initial testing.

FIG. 21 shows the XRPD patterns for 99:1 w:w RBV:PVOH material tested at 6 months.

FIG. 22 depicts a Variability Plot showing Capsule & Device Deposition, Fine Particle Mass and Emitted Dose by NGI Stability data up to DY28 for 30mg RBV Capsules using the Monodose RS01 Device at 60 L/min for 4 seconds (utilizing the crystalline 99:1 w:w RBV:PVOH, 0.9×1 μm cylinder Liquidia PRINT formulation).

FIG. 23 depicts a Variability Plot showing Fine Particle Mass Stability data up to DY28 for 30mg RBV Capsules using the Monodose RS01 Device at 60 L/min for 4 seconds (utilizing the crystalline 99:1 w:w RBV:PVOH, 0.9×1 μm cylinder PRINT formulation).

FIG. 24 shows a Variability Plot showing Fine Particle Fraction Stability data up to DY28 for 30mg RBV Capsules using the Monodose RS01 Device at 60 L/min for 4 seconds (utilizing the crystalline 99:1 w:w RBV:PVOH, 0.9×1 μm cylinder PRINT formulation).

FIG. 25 depicts a Variability Plot showing Emitted Dose Stability data up to DY28 for 30mg RBV Capsules using the Monodose RS01 Device at 60 L/min for 4 seconds (utilizing the crystalline 99:1 w:w RBV:PVOH, 0.9×1 μm cylinder Liquidia PRINT formulation).

FIG. 26 depicts Stage Deposition Plot showing deposition difference for a crystalline RBV PRINT formulation (99:1 w:w RBV:PVOH, 0.9×1 μm cylinder) after storage of capsules naked (without overwrap) at 5° C./Amb and 25° C./60% RH when tested by the same Monodose RS01 device type and flow rate (60 L/min for 4 seconds).

FIG. 27 is a Variability Plot showing Capsule & Device Deposition, Fine Particle Mass and Emitted Dose by NGI Stability data up to DY28 for 30mg RBV Capsules using the Monodose RS01 Device at 60 L/min for 4 seconds (utilizing the crystalline 99:1 w:w RBV:PVOH, 0.9×1 μm cylinder Liquidia PRINT formulation).

FIG. 28 is a Stage Deposition Plot showing difference between crystalline (99:1 w:w RBV:PVOH, 0.9×1 μm cylinder) and amorphous (35:55:10 w:w:w RBV:Trehalose:Trileucine, 0.9×1 μm cylinder) Liquidia PRINT formulations when tested by the same Monodose RS01 device type and flow rate (60 L/min for 4 seconds).

FIG. 29 shows NGI deposition data for 99:1 w:w RBV:PVOH (crystalline) after 2 months of storage at various conditions (bars from left to right in the graph for each NGI stage represent samples stored for 2 months at (1) −20 degrees C.; (2) 25° C./60% RH in a closed container; (3) 25° C./60% RH in an open container, and; (4) 40° C. N=2, except for −20° C. condition.

FIG. 30 depicts DSC data for 99:1 w:w Ribavirin:PVOH (crystalline) on 2 month stability. This data shows no change in crystalline form after 2 months regardless of tested storage conditions (−20° C.; 25° C./60% RH in a closed container; 25° C./60% RH in an open container; and 40° C.).

FIG. 31 shows NGI deposition data for 97:3 w:w RBV:PVOH after 6 months of storage under various conditions (bars from left to right in the graph for each NGI stage represent samples stored for 2 months at (1) −20° C. in a closed container; (2) 25° C./60% RH in a closed container; (3) 25° C./60% RH in an open container, and; (4) 40° C. closed. N=2, except for -20° C. condition.

FIG. 32 depicts DSC data for 97:3 w:w Ribavirin:PVOH (crystalline) material after 6 month stability under which samples stored at (1) −20° C. in a closed container; (2) 25° C./60% RH in a closed container; (3) 25° C./60% RH in an open container, or (4) 40° C. closed.

FIG. 33 depicts the Composite Mean Plasma and Lung Concentrations of Ribavirin Following a Single Inhalation Administration of a Spray Dried Formulation of Ribavirin at a Target Inhaled Dose of 2 mg/kg to Male Rats.

FIG. 34 depicts a comparative lung concentration profiles (normalized by doses determined gravimetrically) in Winstar Hannover rats in a study of various RBV PRINT formulations, vs. Micronized RBV and Spray dried RBV materials from data presented in Table 14.

FIG. 35 depicts comparative plasma concentration profiles in Winstar Hannover rats (normalized by doses determined gravimetrically) in a study of various RBV PRINT formulations, vs. Micronized RBV and Spray dried RBV materials from data in Table 14.

FIG. 36 depicts the fold change in dose-normalized rat lung Cmax and AUC compared to micronized RBV blended in lactose carrier from data presented in Table 14.

### DETAILED DESCRIPTION OF THE INVENTION

The present inventions now will be described more fully hereinafter with reference to the accompanying drawings, in which some, but not all embodiments of the inventions are illustrated. Indeed, these inventions may be embodied in many different forms and should not be construed as limited to the embodiments set forth herein. As used in the specification, and in the appended claims, the singular forms “a”, “an”, “the”, include plural referents unless the context clearly dictates otherwise.

The following terms, as used herein, have the meanings indicated: “Amino acid” refers to α-amino acids such as glycine, alanine, valine, leucine, isoleucine, methionine, proline, phenylalanine, trytophan, serine, threonine, cysteine, tyrosine, asparagine, glutamic acid, lysine, arginine, histidine, norleucine, and modified forms thereof.

“Amorphous” means disordered arrangements of molecules without a distinguishable crystal lattice.

“AUC” or “the area under the curve” is the area under the curve in a plot of concentration of drug in a given fluid, e.g., blood, plasma, lung homogenate or bronchiolar epithelial lining fluid, against time.

“BID” means 2 times a day.

“Bulk Density” refers to the ratio of the mass of an untapped powder sample to its volume including the contribution of the interparticulate void volume. Hence, the bulk density depends on both the density of powder particles and the spatial arrangement of particles in the powder bed. The bulk density is expressed in grams per milliliter (g/mL) although the international unit is kilogram per cubic meter (1 g/mL=1000 kg/m³). It may also be expressed in grams per cubic centimeter (g/cm³).

“Cmax” means the maximum (or peak) concentration of a drug in a given fluid (e.g., plasma) that is achieved in a specified compartment or test area of the body after the drug has been administrated and prior to the administration of a second dose.

“Crystals” possess different arrangements and/or conformations of the molecules in a crystal lattice.

“Dry powder” refers to a powder composition that typically contains less than about 20% moisture, preferably less than 10% moisture, more preferably contains less than about 5% moisture, and most preferably contains less than about 3% moisture, depending upon the particular formulation.

“Dry powder inhaler” or “DPI” means a device containing one or more doses of the pharmaceutical composition of the present invention in dry powder form, a mechanism for exposing a dose of the dry powder into an air flow, and an outlet, in the form of a mouthpiece, through which a user may inhale the pharmaceutical composition.

“ELF” means “epithelial lining fluid” (i.e., lung mucus).

“Emitted Dose” or “ED” provides an indication of the delivery of a drug formulation from a suitable inhaler device after a firing or dispersion event. More specifically, for dry powder formulations, the ED is a measure of the percentage of powder which is drawn out of a unit dose package and which exits the mouthpiece of an inhaler device. The ED is defined as the ratio of the dose delivered by an inhaler device to the nominal dose, i.e., the mass of powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered Dose Uniformity, Sampling the Delivered Dose from Dry Powder Inhalers, United States

Pharmacopeia convention, Rockville, Md., 13th Revision, 222-225, 2007. This method utilizes an in vitro device set up to mimic patient dosing.

“Fabricated particle” means an intentionally molded particle formed of an active pharmaceutical ingredient and optionally further comprising one or more excipients.

“Fine particle fraction” or “FPF” is the mass of drug entering the respiratory tract during inhalation that is contained in aerosol particles between two chosen aerodynamic diameters. Inhaled fine particle fraction is the ratio of inhaled fine particle mass to mass of drug inhaled as aerosol.

“Fine Particle Mass” or “FPM” when referring to a Next Generation Impactor (NGI) is the sum of stages 3, 4 & 5 at 60 L/min that incorporates the 0.94-4.46 μm range.

“IAD”=“immediately after dosing”,

“Leucine”, whether present as a single amino acid or as an amino acid component of a peptide, refers to the amino acid leucine, which may be a racemic mixture or in either its D- or L-form.

“Lung”, in reference to a sample, refers to whole lung homogenate and epithelial lining fluid.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated.

“Metered dose inhaler” or “MDI” means a unit comprising a can, a secured cap covering the can and a formulation metering valve situated in the cap. MDI system includes a suitable channeling device. Suitable channeling devices comprise for example, a valve actuator and a cylindrical or cone-like passage through which medicament may be delivered from the filled canister via the metering valve to the nose or mouth of a patient such as a mouthpiece actuator.

“Next Generation Impactor (NGI)” is a cascade impactor for classifying aerosol particles into size fractions, containing seven impaction stages plus a final micro-orifice collector, which is commercially available, for example, from MSP Corporation, Minn., USA. The impactor is described, for example in U.S. Pat. 6,453,758, 6,543,301, and 6,595,368; UK Patent GB2351155, GB2371001, and GB2371247.

“Non-spherical” refers to a shape, which is other than a sphere or spheroid.

“Pharmaceutically acceptable carrier/diluent” refers to materials that may optionally be included in the compositions of the invention, and taken into the lungs with no significant adverse toxicological effects to the subject, and particularly to the lungs of the subject. Pharmaceutically acceptable carrier/diluent may include one or more materials, which improve the chemical or physical stability of the composition.

“Pharmacologically effective amount” or “physiologically effective amount of a bioactive agent” is the amount of an active agent present in an aerosolizable composition as described herein that is needed to provide a desired level of active agent in the bloodstream or at the site of action (e.g., the lungs) of a subject to be treated to give an anticipated physiological response when such composition is administered to the lungs. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein.

“Polymorphic forms” or “Polymorph” means different crystalline forms as well as solvates and hydrates.

“Polypeptide(s)” refers to multiple bonded amino acids, which may include dimers or trimers of bonded amino acids or even longer chains of bonded amino acids. For example, in some embodiments, for di-leucyl containing trimers, the third amino acid component of the trimer is one of the following leucine (leu), valine (val), isoleucine (isoleu), tryptophan (try) alanine (ala), methionine (met), phenylalanine (phe), tyrosine (tyr), histidine (his), or proline (pro).

“Ribavirin” means, 1-β-D-ribofuranosyl-1H-1,2,4-triazole-3-carboxamide or 1-((2R,3R,4S,5R)-3,4-dihydroxy-5-(hydroxymethyl)tetrahydrofuran-2-yl)-1H-1,2,4-triazole-3-carboxamide, and has the following structural formula:

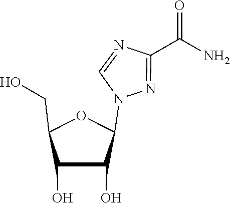

Ribavirin has been designated as CAS Number 36791-04-5. Its empirical formula (Hill Notation) is C₈H₁₂N₄O₅, and it has a molecular weight of 244.20 g/mole. Ribavirin is identified as MDL number MFCD00058564 and is commercially available from Sigma-Aldrich, as compound no. R9644. Ribavirin synthesis is described in Witkowski J.T., et al., Design, synthesis, and broad spectrum antiviral activity of 1-β-D-ribofuranosyl-1,2,4-triazole-3-carboxamide and related nucleosides. J. Med. Chem. 1972 Nov; 15(11):1150-1154.

“Solvates” means crystal forms containing either stoichiometric or nonstoichiometric amounts of solvents. If the incorporated solvent is water, it is referred to as a “hydrate”.

“Tapped Density” means bulk density of a powder (or granular solid) after consolidation/compression prescribed in terms of “tapping” the container of powder a measured number of times, usually from a predetermined height. The method of “tapping” is best described as “lifting and dropping”. Tapping in this context is not to be confused with tamping, sideways hitting or vibration.

“TID” means three times a day, and “QID” means 4 times a day.

“Tmax” means the time after administration of a drug when the maximum concentration is reached in a particular fluid, e.g., blood, plasma, ELF, or lung.

As described herein, applicants have invented a novel pharmaceutical composition comprising a plurality of solid-phase fabricated particles comprising ribavirin and optionally one or more excipients.

Generally, the particles of the present invention are fabricated through the PRINT® Technology (Liquidia Technologies, Inc., Morrisville, N.C., USA). In particular, the PRINT® (Particle Replication In Non-Wetting Templates) particles are made by molding the materials intended to make up the particles in mold cavities. The molds can be polymer-based molds and the mold cavities can be configured to have desired shapes and dimensions. Uniquely, as the particles are formed in the cavities of the mold, the particles are highly uniform with respect to shape, size and composition. The methods and materials for fabricating the particles of the present invention are further described and disclosed in the following issued patents and co-pending patent applications, each of which are incorporated herein by reference in their entirety: U.S. Pat. Nos. 8,944,804, 8,465,775; 8,263,129; 8,158,728; 8,128,393; 7,976,759; 8,444,907; and U.S. Pat. Application Publications Nos. 2013-0249138; US 2012-0114554; and US 2009-0250588, and Rolland J., et al. “Direct Fabrication and Harvesting of Monodisperse, Shape-Specific Nanobiomaterials” J. Am. Chem. Soc. 2005, 127, 10096-10100; each of which are incorporated herein by reference in their entirety.

The fabricated particles are generally non-spherical, having an engineered shape corresponding to a mold in which the particles are formed. The fabricated particles are therefore substantially uniform in shape, substantially equal size, or substantially uniform in shape and substantially equal size. The uniformity is advantageously monodisperse in comparison with other milled, or spray dried materials which will possess a variety of aerodynamically sized and shaped particles.

A further attribute of the fabricated particles of the invention is that in their physical structure, they may be generally homogeneously solid throughout. Thus, they lack hollow cavities or large porous structures which may be created in droplet creation and liquid phase evaporative processes, for example spray drying. This homogeneity allows for densification of the materials within the particles, which may provide such benefits such as compositional rigor, increased drug loading per fabricated particle, and the like.

In one or more embodiments of this aspect of the invention, the fabricated particles may be substantially non-porous.

Molding potentially allows the fabricated particles to be formed in a wide variety of shapes, including but not limited to: those having two substantially parallel surfaces; two substantially parallel surfaces with each substantially parallel surface having substantially equal linear dimensions; two substantially parallel surfaces and one or more substantially non-parallel surfaces. Other shapes include those with one or more angle, edge, arc, vertex, and/or point, and any combination thereof. Other shapes when viewed in 2-dimensions, in a particular orientation, may include triangles, trapezoids, rectangles, arrows, quadrilaterals, polygons, circles, and the like. In 3-dimensions, the shapes may include cones, cubes, cuboidals, prisms, pyramids, polyhedrons, and cylinders, whether right, truncated, frustum, or oblique.

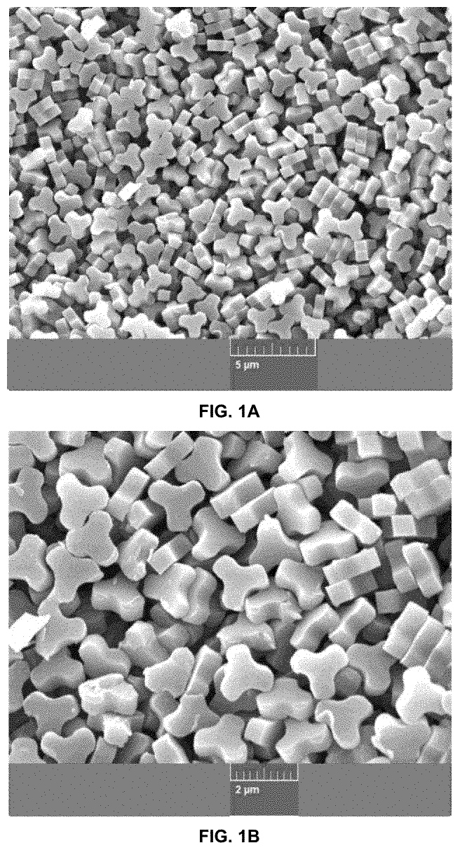

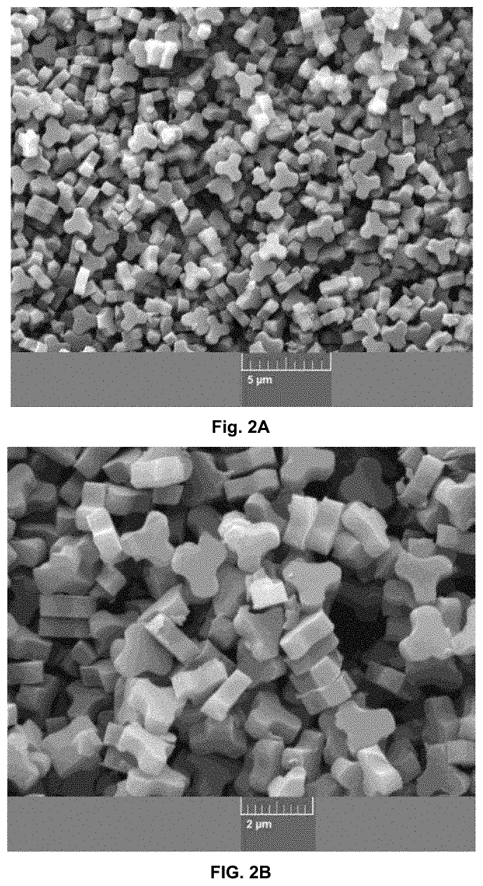

In some embodiments, the shape may comprise when viewed from a top-plan view, a multi-spoked particle, where a plurality of the spokes radiate generally in a single plane from a central point, each spoke forming an arm. For example, see FIG. 1A, FIG. 1B, FIG. 2A and FIG. 2B which depict fabricated particles of the instant invention having a “pollen” configuration, wherein the particle, when viewed in top-plan view, comprises a three spoked particle, where the spokes are arms each emanating from a central point, and extending in a common plane. In particular embodiments the arms of the particle are equidistant from one another, i.e. distributed symmetrically about the central point. The pollen shape also provides a shape wherein a majority of the surface area of the particle comprises substantially planar and substantially parallel surfaces. In other embodiments, the shape may comprise a cylindrical shape having two substantially parallel surfaces; i.e., top and a bottom substantially parallel surfaces.

The “pollen”-shaped particles depicted in FIG. 1A, FIG. 1B, FIG. 2A and FIG. 2B, have a broadest dimension of about 3 μm, and if viewed in side-view, wherein the top surface and bottom surface are generally parallel, the height of such a particle is less than 1 μm, such as between 0.55 μm and 0.65 μm.

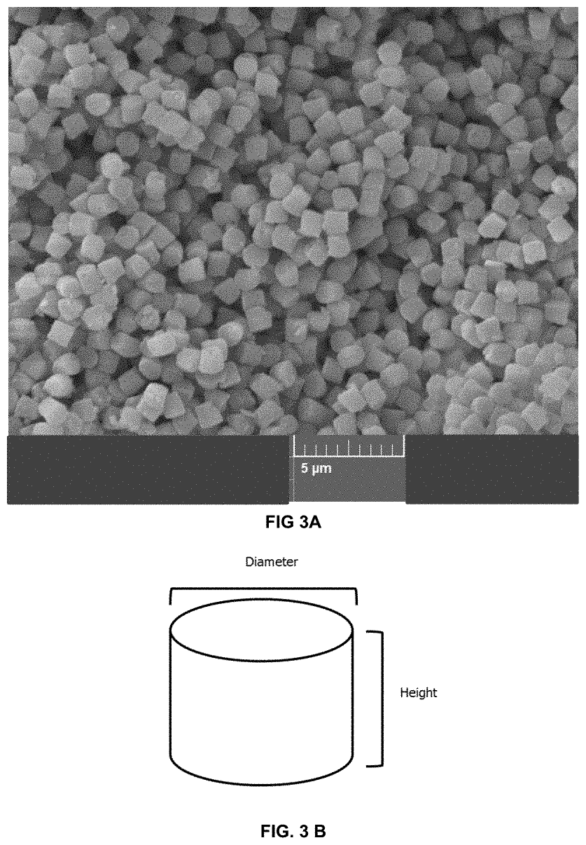

In some embodiments, the fabricated particles are substantially cylindrical shaped. Cylindrical shaped particles are depicted in FIG. 3A and FIG. 3B. As shown in FIG. 3B, the cylinder may be described in terms of two dimensions: the diameter and the height. In some embodiments, the diameter is greater than the height. In other embodiments, the diameter is less than the height. The diameter of the cylindrical particle may be about 5 μm or less, such as 4.5 μm, 4.0 μm, 3.5 μm, 3.0 μm, 2.5 μm, 2.0 μm, 1.5 μm, 1.0 μm, 0.9 μm, 0.8 μm, 0.7 μm, 0.6 μm, 0.5 μm, 0.4 μm, 0.3 μm, or 0.2 μm. In particular embodiments, the diameter of the cylindrical particle is approximately 0.9 μm. The height of the cylindrical particle may be about 5 μm or less, such as 4.5 μm, 4.0 μm, 3.5 μm, 3.0 μm, 2.5 μm, 2.0 μm, 1.5 μm, 1.0 μm, 0.9 μm, 0.8 μm, 0.7 μm, 0.6 μm, 0.5 μm, 0.4 μm, 0.3 μm, or 0.2 μm. In particular embodiments, the height of the cylindrical particle is approximately 1.02 μm.

Thus, the fabricated particles comprise, in cross section, two substantially parallel surfaces (sides), wherein thickness of the particles (between the parallel surfaces (sides)) is about 5.0 μm or less, such as 4.5 μm, 4.0 μm, 3.5 μm, 3.0 μm, 2.5 μm, 2.0 μm, 1.5 μm, 1.0 μm, 0.75 μm, 0.5 μm to approximately 0.25 μm. In some embodiments, fabricated particles of the invention include those having a shape which, when observed in side-view, has a width which is greater than the height of the particle, the height of the shape being defined between the top surface and the bottom surface of the particle, the top and bottom surfaces being substantially parallel to each other, and wherein thickness of the particles between the top and bottom surfaces is less than or equal to about 6 μm, such as less than or equal to about, independently, 4.5 μm, 4.0 μm, 3.5 μm, 3.0 μm, 2.5 μm, 2.0 μm, 1.5 μm, 1.0 μm, 0.75 μm, 0.5 μm or 0.25 μm. In some embodiments, the height of the particle is about 2 μm or less. In some embodiments, the height of the particle is less than 1 μm, such as, between about 0.25 μm and about 0.75 μm.

In other embodiments, fabricated particles of the invention include those having a shape which, when observed in side-view, has a height which is greater than the width of the particle, the height of the shape being defined between the top surface and the bottom surface of the particle, the top and bottom surfaces being substantially parallel to each other, and wherein thickness of the particles between the top and bottom surfaces is less than or equal to about 6 μm, such as less than or equal to about, independently, 4.5 μm, 4.0 μm, 3.5 μm, 3.0 μm, 2.5 μm, 2.0 μm, 1.5 μm, 1.0 μm, 0.75 μm, 0.5 μm or 0.25 μm. In some embodiments, the height of the particle is about 2 μm or less. In some embodiments, the height of the particle is approximately 1 μm, such as, between about 0.90 μm and about 1.1 μm.

As will be appreciated, aerosolized particles deposit in the lung dependent upon aerodynamic factors, as well as on other factors such as density, air flow velocity and directionality, among others. Aerodynamically, the fabricated particles of the present invention are designed to be less than about 7 μm, but larger than about 0.5 μm in size. Thus, they are designed to deposit in the central and/or peripheral airways. Thus, fabricated particles may be molded to have an aerodynamic size, independently, of less than 7 μm, 6 μm, 5 μm, 4 μm, 3 μm, 2 μm, or 1 μm.

The pharmaceutical compositions of the present invention are intended for delivery to the lung and will possess a mass median aerodynamic diameter (MMAD) of less than about 7 μm, for example, from about 6 μm to about 0.5 μm. Thus, compositions of such fabricated particles may have a MMAD of less than, independently, 7 μm, 6 μm, 5 μm, 4 μm, 3 μm, 2 μm, or 1 μm.

In some embodiments, each of the fabricated particles of the composition comprises an amount of ribavirin ranging from about 0.04 picograms to about 4.5 picograms, such as, from about 0.40 picograms to about 0.45 picograms.

The percentage of ribavirin present in the fabricated particles is dependent on a number of factors, such as the amount of ribavirin which is desired to be administered, the given volume of the composition which is required to be delivered, and the particle composition. However, in one or more embodiments of the invention, the percentage loading of ribavirin ranging from about 1% w/w to greater than 99% w/w of the fabricated particles. Representative of this are fabricated particles comprising a percentage loading of ribavirin 5% w/w to about 99.5% w/w; 10% w/w to about 99.5% w/w; from about 10% w/w to about 55% w/w; from about 20% w/w to about 50% w/w; or a range of from about 30% w/w to about 40% w/w.

Thus, in various embodiments of the invention, the percentage loading of ribavirin in the fabricated particles is greater than about 1.0% w/w, such as greater than 5.0% w/w, such as greater than about 10%, 15%, 20%, 25% 30%, 35%, 40%, 45%, 50%, 55%, 60%, 65%, 70%, 75%, 80%, 85%, 90%, 95%, 96%, 97%, 98%, and 99% w/w fabricated particles.

In certain embodiments, the percentage loading of ribavirin in the fabricated particles is greater than or equal to 95% w/w. In certain embodiments, the percentage loading of ribavirin in the fabricated particles is greater than or equal to 97% w/w. In certain other embodiments, the percentage loading of ribavirin in the fabricated particles is greater than or equal to 99% w/w.

Thus, the range of ribavirin to excipient in the present invention may be from about 1% to 99.5% ribavirin, to from 99% to 0.5% excipient.

In one or more embodiments of the invention, the ribavirin in the fabricated particles is amorphous or substantially amorphous. In other embodiments of the invention, the ribavirin in the fabricated particles is crystalline or substantially crystalline.

Ribavirin is known to have two polymorphic crystalline forms. See Prusiner, P., et al., “The crystal and molecular structures of two polymorphic crystalline forms of virazole (1-β-D-ribofuranosyl-1,2,4-triazole-3-carboxamide). A new synthetic broad spectrum antiviral agent”, Acta Cryst. (1976), B32, 419-426. Crystalline ribavirin is a white powder freely soluble in water and slightly soluble in ethanol (96%). It has a specific optical rotation between −33.0° and −37.0° (dried basis 10 mg/ml, t=20° C.), pH 4.0-6.5. Ribavirin may exist in two polymorphic crystalline forms, II and I that are distinguishable by their melting range. One form crystallized from aqueous ethanol, Form II which is thermodynamically stable at room temperature, melts at about 166˜168° C. (thermostable Form II), while the other form (Form I) crystallized from ethanol, is metastable at room temperature, and has a melting range of 174˜176° C. In embodiments, crystalline Form II is preferred.

In one embodiment, the present invention incorporates a percentage of Form II ribavirin within the particles plus, optionally, one or more excipients described herein, or alternatively, 90% or greater, 95% or greater, 96%, 97%, 98%, 99%, or 100% of the entire particle is made of Form II ribavirin. In certain embodiments, the particles are 99% Form II ribavirin with the remaining 1% of the particle comprising polyvinyl alcohol (PVOH). In other embodiments, the particles are 100% Form II ribavirin.

In another embodiment, the present invention incorporates a percentage of Form I ribavirin within the particles plus one or more excipients described herein, or io alternatively, 90% or greater, 95% or greater, 96%, 97%, 98%, 99%, or 100% of the entire particle is made of Form I ribavirin. In certain embodiments, the particles are 99% Form I ribavirin with the remaining 1% of the particle comprising polyvinyl alcohol (PVOH). In other embodiments, the particles are 100% Form I ribavirin.

The fabricated particles of the present invention may also comprise varying mixtures (or ratios) of crystalline Form I ribavirin, crystalline Form II ribavirin, and/or amorphous ribavirin and optionally also comprise one or more excipients described herein or may be substantially free of any excipients. For example, the fabricated particles of the present invention in one embodiment may substantially comprise Form II ribavirin, wherein less than 10%, less than 5%, or less than 1% of the ribavirin is Form I and/or amorphous ribavirin.

### Crystalline Polymorph Control

A material may exist in different crystalline forms, each being a polymorph. A material may exist in a single crystalline form, or may have two, three, four, or more polymorphs. Although polymorphs have the same chemical composition, polymorphs potentially have different physical and/or chemical properties. For example, the polymorphs may have different physical and/or chemical properties. Polymorphs may have different solubility, dissolution rates, physical stability, chemical stability, melting points, colors, density, flow characteristics, safety, efficacy, and/or bioavailability. Additionally, polymorphs may convert from one form to another during manufacturing and/or storage, for example from the metastable form to the thermostable form. This form conversion may or may not be desirable. Consequently, polymorphs impact pharmaceutical CMC (Chemistry, Manufacturing, and Controls) and regulatory requirements. Therefore, there is a desire to produce a specific polymorph form or forms.

There are various techniques available to achieve and control polymorph formation and growth. While some are described herein, it will be readily recognized to those skilled in the art that alternatives to those discussed are available, and are considered within the scope of the present invention. Some non-limiting examples include use of a saturated or supersaturated solution and temperature control. A saturated or supersaturated solution may provide multiple points of nucleation leading to crystal formation. Crystals formed may or may not be the desired polymorph. If a specific polymorph is desired, use of crystal seeds (seeding) with the desired polymorph form will encourage the formation of crystals having the desired form. If a less desirable polymorph is formed, the crystals may be further treated to convert the undesirable polymorph to the desired polymorph.

When producing particles within molds, such as in PRINT® Technology, it may be useful and in some instances important to have crystals (seeds) present in some or each mold cavity or within communication with the mold cavities to initiate the crystallization process in the cavities. In some embodiments, the mold cavities are left in communication with each other through the use of a flash layer of the substance to be molded interconnecting the cavities. In alternative embodiments seeds are transferred to the mold cavities when the stock solution used to prepare the particles is dispensed into the mold. The stock solution may be filtered using a filter having a pore size sufficiently large to allow the passage of crystals of the active agent (i.e. such as a drug like Ribavirin). Alternatively, the stock solution may be used for fabrication of particles without filtering. In further embodiments, the stock solution is filtered before addition of the active agent, then utilized in PRINT® Technology molding processes, described herein, before the active agent fully dissolves such that crystals of the active agent remain in the stock solution introduced into the PRINT® Technology molding process.

Temperature may be varied during the crystallization process. The temperature may be varied during the formation of crystals or after the crystals are formed. For example, the temperature may be cycled, involve a specific temperature exposure schedule, or comprise exposure of the material for a specified temperature range for a specified time interval. Additional conditions during temperature control may also be critical to polymorph formation. For example, during or after crystallization, crystals may annealed when stored under specific temperature and relative humidity conditions for specific time duration.

Annealing may be employed to promote crystalline form formation and growth. Annealing may be conducted in an annealing chamber. Annealing chamber conditions can be highly influential on the time to crystallize. Annealing chamber conditions may also impact particle surface quality. Dependent on annealing conditions selected, a potential may exist for the formation of polycrystalline domains in the particles. Polycrystalline domains may lead to fracture which in turn impacts surface quality and therefore aerosolization performance.

Differential scanning calorimetry (DSC) has shown useful in understanding the presence of crystalline forms. Regarding ribavirin, DSC has been useful in understanding the level of Form I and/or Form II in manufactured materials. The ratio of enthalpies for the melting endotherms of Form I and Form II (ΔHm, Form I/ΔHm, Form II) is an indication of the form purity for a particular sample. Utilizing PRINT® Technology, samples comprising ΔHm, Form I/ΔHm, Form II less than 5% have been produced. In preferred embodiments, ΔHm, Form I/ΔHm, Form II is less than 1%.

Compositions of particles of the present invention may be characterized in terms of bulk density. The bulk density of the pharmaceutical compositions of the present invention are generally less than about 3.0 g/cm³, for example less than 2.5 g/cm³, less than or equal to 2.0 g/cm³, less than 1.5 g/cm³, and less than 1.0 g/cm³.

Ribavirin containing particles of the present invention may also comprise one or more excipients. Suitable excipients of the fabricated particles may include one or more carbohydrate, amino acid/polypeptide, natural polymers, or synthetic polymers, alone, or in any combination. With certain embodiments, the carbohydrate excipients may include for example:
monosaccharides, such as dextrose (anhydrous and monohydrate), fructose, galactose, maltose, D-mannose, sorbose, and the like; disaccharides, such as lactose, maltose, sucrose, trehalose, cellobiose, and the like;
polysaccharides, such as raffinose, melezitose, maltodextrins, dextrans, starches, and the like; and
alditols, such as mannitol, xylitol, maltitol, lactitol, xylitol, sorbitol (glucitol), pyranosyl sorbitol, myoinositol and the like.

In some embodiments, the disaccharide is lactose or trehalose or a combination of lactose and trehalose. In some embodiments, the disaccharide is trehalose.

In some embodiments, the polysaccharide is dextran having a molecular weight between about 1,000 g/mole and 20,000 g/mole. One particular polysaccharide is dextran having a molecular weight of about 6,000 g/mole.

Non-limiting examples, of amino acid/polypeptide components which are suitable for use in the present invention include, but are not limited to, alanine, glycine, arginine, betaine, histidine, glutamic acid, aspartic acid, cysteine, lysine, leucine, isoleucine, valine, methionine, phenylalanine, aspartame, tyrosine, tryptophan, and the like. In certain embodiments, the amino acids are hydrophobic amino acids, such as leucine, valine, isoleucine, tryptophan, alanine, methionine, phenylalanine, tyrosine, histidine and proline. One suitable amino acid is the amino acid leucine. Leucine, when used in the formulations described herein includes D-leucine, L-leucine, and mixtures thereof, including racemic leucine.

Polypeptides including dimers, trimers, tetramers, and pentamers composed of hydrophobic amino acid components such as those described above are also suitable for use. Examples include di-leucine, di-valine, di-isoleucine, di-tryptophan, di-alanine, and the like; trileucine, trivaline, triisoleucine, tritryptophan etc.; mixed di- and tri-peptides, such as leucine-valine, isoleucine-leucine, tryptophan-alanine, leucine-tryptophan, etc., and leucine-valine-leucine, valine-isoleucine-tryptophan, alanine-leucine-valine, and the like and homo-tetramers or pentamers such as tetra-alanine and penta-alanine. In one embodiment, the polypeptide is trileucine.

Synthetic polymers include, but are not limited to, polyvinyl alcohol (PVOH), polyethylene, polyester, polyethylene terephthalate, polypropylene, low-density polyethylene and high-density polyethylene. In some embodiments the synthetic polymer is PVOH with a molecular weight between about 6,000 g/mole and 30,000 g/mole and has a percent hydrolysis of between about 75 and 90%. A preferred synthetic polymer is PVOH with a molecular weight of about 6,000 g/mole and a percent hydrolysis of 78%. A preferred synthetic polymer is PVOH with a viscosity of 4.8-5.8 mPa·sec (4% aqueous solution at 20° C.) and a degree of hydrolysis of 86.5-89.0%.

Natural polymers include, but are not limited to, collagen, chitosan, gelatin, starch, chitin, pectin, DNA, cellulose and proteins.

The amounts of the various excipients will be dependent of the amount of ribavirin present, as well as the particular excipients used.

As will be described below, in various embodiments of the invention the fabricated particles comprise ribavirin and excipients, e.g., an amino acid and a carbohydrate, representative examples including but not limited to: trileucine and trehalose; or a polysaccharide and/or a synthetic polymer, e.g., dextran and/or PVOH.

In one or more embodiments, the excipient is selected from a group consisting of trehalose, trileucine, dextran, and lactose, alone or in any combination.

In one or more embodiments, the excipient is lactose. In such embodiments the percentage of lactose is between 40 and 80 percent of the particles, e.g. 65 percent.

In one or more still further embodiments, the excipient includes or is trehalose. For example, in such embodiments, the percentage of trehalose in such particles is between 40 and 80 percent, e.g., 65 percent.

In still further embodiments, the excipient includes or is trileucine. In certain such embodiments, the percentage of trileucine is between about 5 and about 20 percent, e.g., 15 percent.

In one or more embodiments, the excipient is PVOH. For example, in such embodiments, the percentage of PVOH in such particles is between about 0 and about 5 percent, e.g. 1 percent.

In certain embodiments, in this aspect of the invention, the percentage of ribavirin is between 25 and 100 percent of the particles, e.g., 95-99 percent.

In one or more particular embodiments, the particles comprise ribavirin, trehalose and trileucine. In one or more of such embodiments, the composition comprises about 55 percent trehalose and about 10 percent trileucine by weight based on solid materials. In one or more embodiments, the excipient is PVOH. In other particular embodiments, the particles comprise ribavirin and PVOH. In one or more of such embodiments, the composition comprises about 95 percent ribavirin and about 5 percent PVOH or about 96 percent ribavirin and about 4 percent PVOH or about 97 percent ribavirin and about 3 percent PVOH or about 98 percent ribavirin and about 2 percent PVOH or about 99 percent ribavirin and about 1 percent PVOH by weight based on solid materials.

Exemplary compositional ranges are contained in the tables below.

TABLE 1
Dextran System

| Component | Weight Percent (Based on Solids) |
|---|---|
| Dextran | 25-99 |
| Ribavirin | 1-75 |
| PVOH | 0.1-1 |

TABLE 2
Trileucine/Trehalose System

| Component | Weight Percent (Based on Solids) |
|---|---|
| Trileucine | 5-15 |
| Trehalose dihydrate | 50-94 |
| Ribavirin | 1-35 |

TABLE 3
Leucine/Lactose System

| Component | Weight Percent (Based on Solids) |
|---|---|
| Leucine | 5-15 |
| Lactose | 50-94 |
| Ribavirin | 1-35 |

TABLE 4
Crystalline System

| Component | Weight Percent (Based on Solids) |
|---|---|
| Ribavirin | 95-99.5 |
| PVOH | 0.5-5.0 |

### Particle Fabrication

Generally, the particles of the present invention are fabricated through PRINT® Technology (Liquidia Technologies, Inc.). In particular, PRINT® Technology uses the following steps: stock solution preparation, particle fabrication, optional annealing, and harvesting. After harvesting, the particles may be packaged and labeled.
1. Stock Solution Preparation: Particle stock solution or particle precursor is prepared. The particle stock solution or particle precursor contains active agent(s) and other materials such as excipients.
2. Particle Fabrication: The particles are made by molding the materials intended to make up the particles (i.e. particle stock solution or particle precursor) in mold cavities. The material to be molded is first cast onto a PET film into controlled thickness and dried. The dried thin film is then married with the PRINT® mold, nipped in a heated nip to urge the composition into the mold cavities. In some embodiments, a flash layer of the particle material remains between the mold and the PET film, thereby connecting each mold cavity in crystallization communication. Thereafter, in some embodiments, the PET film remains in place to effectively close the cavities of the mold for further processing. The molds can be polymer-based molds and the mold cavities can be formed into desired shapes and dimensions. Uniquely, as the particles are formed in the cavities of the mold, the particles are highly uniform with respect to shape, size, and composition.
3. Annealing: The particles may, optionally, be annealed. Annealing involves, for example, storage at a specific temperature and relative humidity for specific time duration. Generally, particles are stored in the mold during the annealing process.
4. Harvesting: When harvested, the particles are removed from the mold. Removal includes separating the PET film that effectively delivered the thin film and closed the mold cavities during annealing. Removal of the PET film generally removes the particles from the mold cavities, wherein the particles are more tightly coupled with the PET film than to the cavities of the mold. Next, the particles are removed from the film. Removal of the particles from the film generally includes scraping, brushing or other process that disrupts the particles from coupling with the film. After harvesting, the particles may be further processed prior to packaging. For example, the particles may be sieved.
5. Labeling and packaging: Once harvested, the particles may be packaged and labeled for bulk storage, packaged in final form for consumer use, or packaged in some intermediate configuration.

The methods and materials for fabricating the particles of the present invention are further described and disclosed in the following issued patents and co-pending patent applications, each of which are incorporated herein by reference in their entirety: U.S. Pat. Nos. 8,944,804; 8,465,775; 8,263,129; 8,158,728; 8,128,393; 7,976,759; 8,444,907; and U.S. Pat. Application Publications Nos. 2013-0249138; and US 2012-0114554.

### Mold Information

Particles of various shape/size configurations can be molded using stock solutions. PRINT® molds used to fabricate these particles are described in the references incorporated herein.

In one approach to this process, the fabricated particles are produced by a process comprising:
a. creating an admixture of ribavirin and optionally one or more excipients;
b. casting the admixture into a film on a backing sheet;
c. nipping the film with a mold in a nip roller, wherein the mold defines mold cavities;
d. flowing the film into the mold cavities where it remains on the backing sheet in isolated fabricated particles; and
e. removing the isolated fabricated particles from the backing sheet.

In another approach to this process, the fabricated particles are produced by a process comprising:
a. creating an admixture of ribavirin and optionally one or more excipients;
b. casting the admixture into a film on a backing sheet;
c. nipping the film with a mold in a nip roller, wherein the mold defines mold cavities;
d. flowing the film into the mold cavities and retaining a flash layer of the film composition interconnecting the mold cavities;
e. annealing the composition in the mold cavities to convert the composition from an amorphous solid to crystalline solid particles mimicking the shape and volume of the mold cavities;
f. separating the backing sheet from the mold and thereby removing the particles from the mold cavities where they remain on the backing sheet in isolated fabricated particles; and
g. removing the isolated fabricated particles from the backing sheet.

In certain embodiments, the fabricated particles containing ribavirin according to the present invention may be formed by a process comprising:
a. mixing a solution of ribavirin and optionally one or more excipients in a solvent;
b. casting the solution into a film on a backing sheet;
c. drying the film on the backing sheet;
d. nipping the dried film with a mold in a heated nip roller, wherein the mold defines mold cavities;
e. flowing the dried film into the mold cavities where it remains on the backing sheet in isolated fabricated particles;
f. removing the isolated fabricated particles from the backing sheet; and
g. collecting the isolated fabricated particles.

In other embodiments, the fabricated particles containing ribavirin according to the present invention may be formed by a process comprising:
a. mixing a solution of ribavirin and optionally one or more excipients in a solvent;
b. casting the solution into a film on a backing sheet;
c. drying the film on the backing sheet;
d. nipping the dried film with a mold in a heated nip roller, wherein the mold defines mold cavities;
e. flowing the dried film into the mold cavities and retaining a flash layer of the film composition interconnecting the mold cavities;
f. annealing the composition in the mold cavities to convert the composition from an amorphous solid to crystalline solid particles mimicking the shape and volume of the mold cavities;
g. separating the backing sheet from the mold and thereby removing the particles from the mold cavities where they remain on the backing sheet in isolated fabricated particles;
h. removing the isolated fabricated particles from the backing sheet; and
i. collecting the isolated fabricated particles.

In certain embodiments, the fabricated particles containing ribavirin according to the present invention may be formed by a process comprising:
a. preparing a particle precursor solution of ribavirin and optionally one or more excipients in a solvent;
b. casting the particle precursor solution into a film on a sheet;
c. drying the film by removing the solvent, retaining the particle precursor on the sheet;
d. nipping the sheet containing the dried particle precursor with a mold in a heated nip roller, wherein the mold defines mold cavities;
e. flowing and solidifying the particle precursor into the mold cavities to create isolated uniform shaped particles of the particle precursor in the mold cavities;
f. removing the sheet from the mold wherein the isolated uniform shaped particles remain on the sheet and are removed from the mold cavities;
g. removing the isolated uniform shaped particles from the sheet; and
h. collecting the isolated uniform shaped particles.

In certain further embodiments, the fabricated particles containing ribavirin according to the present invention may be formed by a process comprising:
a. preparing a particle precursor solution of ribavirin and optionally one or more excipients in a solvent;
b. casting the particle precursor solution into a film on a sheet;
c. drying the film by removing the solvent, retaining the particle precursor on the sheet;
d. nipping the sheet containing the dried particle precursor with a mold in a heated nip roller, wherein the mold defines mold cavities;
e. flowing and solidifying the particle precursor into the mold cavities and retaining a flash layer of the film composition interconnecting the mold cavities;
f. annealing the composition in the mold cavities to convert the composition from an amorphous solid to crystalline solid particles mimicking the shape and volume of the mold cavities;
g. removing the sheet from the mold wherein the isolated uniform shaped particles remain on the sheet and are removed from the mold cavities;
h. removing the isolated uniform shaped particles from the sheet; and
i. collecting the isolated uniform shaped particles.

In certain embodiments, the fabricated particles containing from 1% to 100% of ribavirin according to the present invention may be formed by a process comprising:
a. preparing a particle precursor solution of ribavirin and optionally one or more excipients in a solvent;
b. casting the particle precursor solution into a film on a sheet;
c. drying the film by removing the solvent, retaining the particle precursor on the sheet;
d. nipping the sheet containing the dried particle precursor with a mold in a heated nip roller, wherein the mold defines mold cavities;
e. flowing and solidifying the particle precursor into the mold cavities to create isolated uniform shaped particles of the particle precursor in the mold cavities;
f. removing the sheet from the mold wherein the isolated uniform shaped particles remain on the sheet and are removed from the mold cavities;
g. removing the isolated uniform shaped particles from the sheet; and
h. collecting the isolated uniform shaped particles.

In certain other embodiments, the fabricated particles containing from 1% to 100% of ribavirin according to the present invention may be formed by a process comprising:
a. preparing a particle precursor solution of ribavirin and optionally one or more excipients in a solvent;
b. casting the particle precursor solution into a film on a sheet;
c. drying the film by removing the solvent, retaining the particle precursor on the sheet;
d. nipping the sheet containing the dried particle precursor with a mold in a heated nip roller, wherein the mold defines mold cavities;
e. flowing and solidifying the particle precursor into the mold cavities and retaining a flash layer of the film composition interconnecting the mold cavities;
f. annealing the composition in the mold cavities to convert the composition from an amorphous solid to crystalline solid particles mimicking the shape and volume of the mold cavities;
g. removing the sheet from the mold wherein the isolated uniform shaped particles remain on the sheet and are removed from the mold cavities;
h. removing the isolated uniform shaped particles from the sheet; and
i. collecting the isolated uniform shaped particles.

In other embodiments, the crystalline particles containing from about 95% to 100%, of ribavirin according to the present invention may be formed by a process comprising:
a. preparing a particle precursor solution of ribavirin in a solvent;
b. casting the particle precursor solution onto a sheet;
c. nipping the sheet containing the particle precursor with a mold in a heated nip roller, wherein the mold defines mold cavities;
d. flowing the particle precursor into the mold cavities;
e. crystallizing the particle precursor in the mold cavities to create isolated uniform shaped crystalized particles of the particle precursor in the mold cavities;
f. removing the sheet from the mold to provide isolated uniform shaped crystalized particles of between about 95% and 100% of ribavirin.

In still other embodiments, the crystalline particles containing from about 95% to 100%, of ribavirin according to the present invention may be formed by a process comprising:
c. preparing a particle precursor solution of ribavirin in a solvent;
d. casting the particle precursor solution onto a sheet;
c. nipping the sheet containing the particle precursor with a mold in a heated nip roller, wherein the mold defines mold cavities;
d. flowing the particle precursor into the mold cavities and retaining a flash layer of the film composition interconnecting the mold cavities;
e. crystallizing the particle precursor in the mold cavities to create isolated uniform shaped crystalized particles of the particle precursor in the mold cavities;
f. removing the sheet from the mold to provide isolated uniform shaped crystalized particles of between about 95% and 100% of ribavirin.

In certain embodiments, the temperature of the nip ranges from about 20° C. to about 300° C. In other embodiments, the pressure of the roller ranges from about 5 psi to about 80 psi. In still other embodiments, speed of the roller is greater than 0 ft/min and ranges up to about 25 ft/min.

In some embodiments, crystalline particle fabrication may be achieved as follows:
1. An aqueous solution is prepared by dissolving a wetting agent into water. The appropriate wetting agent is selected for pharmaceutical applications, such as for example PVOH. After the aqueous solution is prepared, the active pharmaceutical ingredient is added. In some embodiments, the active pharmaceutical ingredient (API) is added in powder form, preferably in a crystalline polymorph desired to be achieved in the particles. More particularly, the powder API may be selected from ribavirin in crystalline polymorph Form II having median size 16 micrometers (Aurobindo Pharma).
2. The crystalline API is allowed to incompletely dissolve in the aqueous solution prior to use in the molding process to form particles of the present invention. Importantly the mixture retains at least some Form II not fully dissolved to retain the initial crystalline polymorph form factor. In a particular embodiment of the present invention, the ribavirin powder is in crystalline polymorph Form II when added to the aqueous solution and allowed to incompletely dissolve to retain seeding for polymorph Form II later in processing. Additionally, Form II seed may be added to saturated, filtered stock solutions to control form.

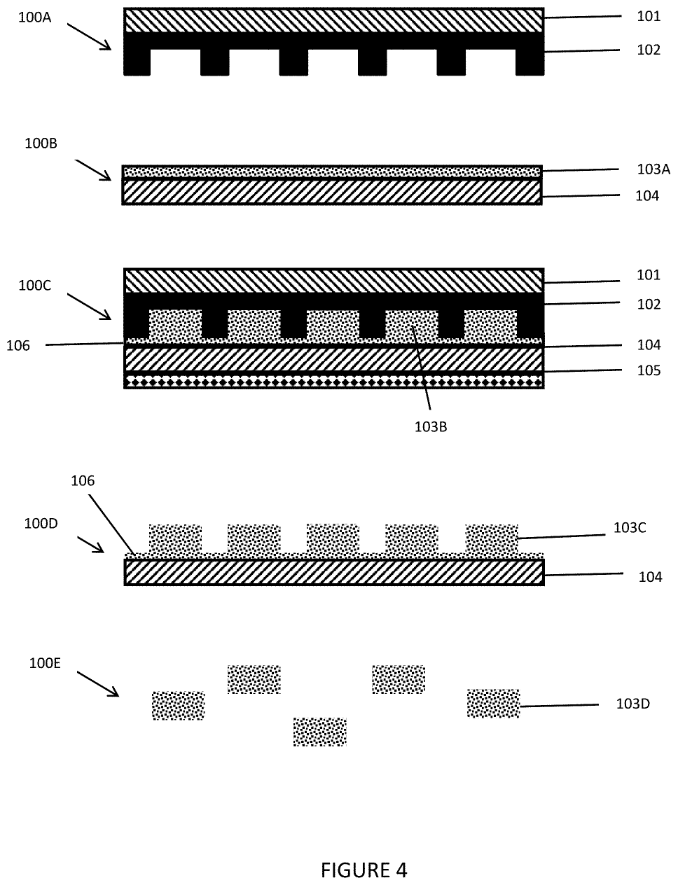

As shown in 1008 of FIG. 4, the aqueous solution with added API, prior to the API being fully dissolved, is dispersed onto delivery sheet 104 and allowed to dry into a thin film 103A. In some embodiments, the thin film is between 500 and 700 nanometers thick. In a preferred embodiment, the thin film is between about 550 nanometers and 650 nanometers thick. Next, the thin film dried on the deliver sheet is married up with the PRINT® mold, shown in 100A, comprising a mold 102 and a mold backing/web 101, to bring the thin film into contact with the mold as the combination passes through a nip point. In some embodiments the nip point is heated and the thin film enters into the cavities in the PRINT® mold. The delivery sheet and mold are generally in contact with the thin film dispersed into and filling the cavities of the mold. In some embodiments, the volume of thin film 103A is determined to be more than the total volume of the mold cavities such that a residual amount, or flash layer 106, of thin film 103A remains between delivery sheet 104 and mold 102. The flash layer 106 can be between 5 nanometers to 75 nanometers thick. In a more preferred embodiment, flash layer 106 can be between 10 nanometers to 50 nanometers thick. During annealing, described elsewhere herein, flash layer 106 provides communication between individual mold cavities 103B for crystallization propagation.

As shown in 100C, the delivery sheet is then overlaid with an interleaf layer 105 and together the delivery sheet, interleaf layer and mold are rolled. The roll is then held under annealing conditions, whereby the material of the thin film now in the mold cavities 103B and flash layer 106 transforms from predominately amorphous composition to the crystalline polymorph form of the seeded API material. After annealing, the mold is separated from the delivery sheet as shown in 100D, thereby leaving particles 103C that mimic the size and shape of the mold cavity and comprise the single polymorph form of API that was incompletely dissolved in the aqueous solution attached to the delivery sheet 104. As shown in 100E, the particles may then be removed from the harvest sheet producing independent, free-standing particles 103D. In preferred embodiments, flash layer 106 is sufficiently thin (i.e. 10 nm to 50 nm) such that upon mold 102 separation from delivery sheet 104 and particle 103C removal from delivery sheet 104 (alternatively called harvest sheet at this stage in the process) flash layer 106 is disrupted and does not interconnect or bind the particles from being free-standing independent particles 103D.

In some embodiments the wetting agent comprises between about 5% and 0.5% of the aqueous solution. In other embodiments, the wetting agent comprises about 1% of the aqueous solution. In a preferred embodiment the wetting agent is PVOH and comprises about 1% of the aqueous solution.

In some embodiments the mold comprises a cavity having a shape corresponding to a cylinder. In some embodiments the cylindrical shape of the mold cavity comprise a diameter of about 1 micrometer and a height of about 1 micrometer. In a preferred embodiment, the diameter of the cylindrical mold cavity is about 0.9 micrometers and the height of the cylindrical mold cavity is about 1 micrometer. According to a particular embodiment of the invention, each of the fabricated particles has a substantially cylindrical shape being between about 0.9 micrometers and 3 micrometers in diameter and between about 1 micrometer and 3 micrometers in height, thus giving the particle an aspect ratio of width to length of between about 0.9:3 and about 3:1.

In some embodiments the interleaf layer provides exposure of the annealing conditions to, or more directly or evenly to, the cavities in the mold to facilitate crystallization in the mold cavities. In some embodiments, the interleaf layer comprises mesh with between about 60% to 70% open area defined by holes between about 0.05 inch by 0.05 inch to about 0.15 inch by 0.18 inch and a thickness of between about 0.019 inch and about 0.035 inch. In a preferred embodiment, the interleaf layer comprises a mesh with about 65% open area defined by holes approximately 0.054 inch by 0.080 inch and a thickness of approximately 0.019 inch.

In some embodiments, the annealing conditions are between about 20 degrees to 60 degrees Celsius and 40-60 percent relative humidity. In other embodiments, the annealing conditions are between about 25 degrees to 40 degrees Celsius and 40-50 percent relative humidity. In more preferred embodiments, the annealing conditions are about 30 degrees Celsius (+/−2 degrees) and 48% relative humidity (+/−5%).

According to a particular embodiment where the particle composition is about 99% ribavirin and 1% PVOH; the mold cavities are cylinders about 0.9 micrometers in diameter and about 1 micrometer in depth; and the interleaf layer defines a mesh with about 65% open area defined by holes approximately 0.054 inch×0.080 inch and a thickness of approximately 0.019 inch, the annealing conditions are about 30 degrees Celsius (+/−2 degrees) and 48 relative humidity (+/−5%) for at least 14 days. Under such conditions the particles resulting in the mold cavities are about 99% ribavirin of which is about 99% crystalline polymorph form II.

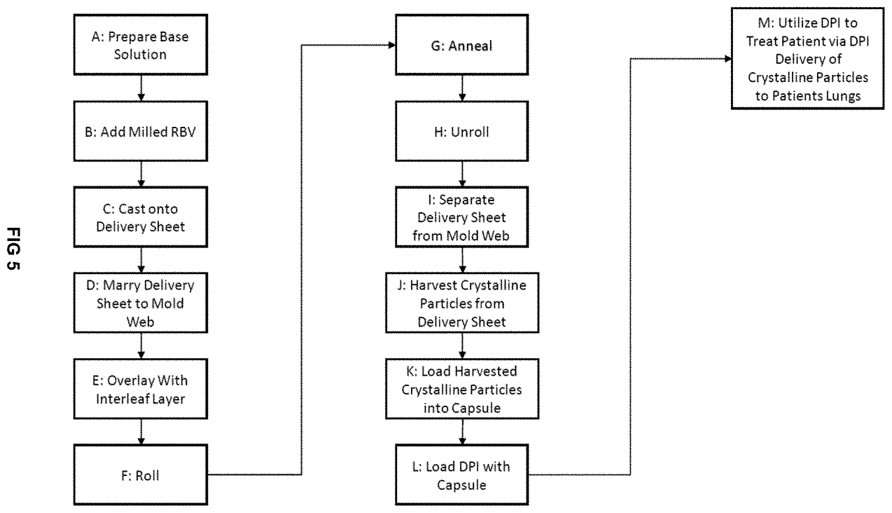

In other embodiments, the particles are fabricated in 1.5 micrometer by 1.5 micrometer cylindrical PRINT® molds. According to such embodiments, a PVOH stock solution is made according to Example 1C having about 0.1 wt % PVOH and between about 10.0 to 11.5 wt % ribavirin. In a more particular embodiment, a stock solution was made according to Example 1C with 0.1075 wt % PVOH and 10.75 wt % ribavirin. Promptly following addition of the ribavirin to the PVOH solution, a thin film is cast on a PET web to a thickness of between about 750 nanometers to about 950 nanometers. The thin film is then mated with the PRINT® mold and the ribavirin solution in the mold cavities is allowed to anneal at 30 (+/−2) degrees C., 48% (+/−5%) relative humidity for about 2 weeks, also as described herein and in Example 1C and FIG. 5 to yield particles mimicking the 1.5 micron by 1.5 micron cylindrical shape mold cavities with a composition that is about 99% crystalline ribavirin polymorph form II.

In further embodiments the particles are fabricated in 3 micrometer by 3 micrometer cylindrical PRINT® molds. According to such embodiments, a PVOH stock solution is made according to Example 1C having about 0.1 wt % PVOH and between about 10.0 to 15 wt % ribavirin. In a more particular embodiment, a stock solution was made according to Example 1C with 0.13 wt % PVOH and 13 wt % ribavirin. Promptly following addition of the ribavirin to the PVOH solution, a thin film is cast on a PET web to a thickness of between about 950 nanometers to about 1350 nanometers. The thin film is then mated with the PRINT® mold and the ribavirin solution in the mold cavities is allowed to anneal at 30 degrees C. (+/−2), 48% relative humidity (+/−5%) for about 2 weeks, also as described herein and in Example 1C and FIG. 5 to yield particles mimicking the 3 micron by 3 micron cylindrical shape mold cavities with a composition that is about 99% crystalline ribavirin polymorph form II.

In certain embodiments of the present invention, fabrication conditions are important to the resulting polymorph form control in the particles. In a particular embodiment, as shown in FIG. 5, Step B includes the conditions of adding powdered crystalline ribavirin in polymorph form II (Aurobindo Pharma) having median particulate size of 16 micrometers, in the amount between about 10-20 percent by weight of the PVOH stock solution (Example 1C), to the PVOH stock solution and stirring for between about 15 to 30 minutes at about 65 degrees C. The mixture from Step B is then promptly transferred onto the delivery sheet and cast into the thin film and dried, Step C. Following casting and drying the thin film, the thin film on the delivery sheet is married with a PRINT® mold (Liquidia Technologies, Inc., Morrisville N.C.) and passed through a nip point to transfer the composition of the thin film into mold cavities of the mold while retaining a flash layer of the solution in crystallization communication with the mold cavities, Step D.

Importantly, Steps B-C of FIG. 5 are accomplished in less than 12 hours, more preferably in less than 5 hours, less than 2 hours and preferably in less than about 90 minutes to yield particles mimicking the shape of the PRINT® mold cavities and having greater than 95% ribavirin polymorph form II. In some embodiments, conditions for processing Steps C and D include ambient room temperature and humidity. In more preferred embodiments, conditions for processing Steps C and D include temperature between about 15-25 degrees C. and relative humidity about 30% (+/−20%).

In another embodiment, it is important that Steps B-F of FIG. 5 are accomplished in less than about 12 hours, more preferably in less than about 10 hours, and more preferably in less than about 3 hours of adding the ribavirin to the PVOH stock solution to yield particles that mimic the shape of the PRINT® mold cavities and have greater than 95% ribavirin polymorph form II.

According to an embodiment of the present invention, Step G occurs in at least 14 days.

According to an embodiment of the present invention, precise shape controlled particles mimicking the shape and size of PRINT® mold cavities comprised of greater than 95% ribavirin, of which is greater than 95% polymorph Form II, are formed through an amorphous solid to crystalline solid crystallization process in less than 15 days. According to more preferred embodiments of the present invention, precise shape controlled particles mimicking the shape and size of PRINT® mold cavities comprised of greater than 97% ribavirin, of which is greater than 99% polymorph Form II, are formed through an amorphous solid to crystalline solid crystallization process in less than 15 days. According to more preferred embodiments of the present invention, precise shape controlled particles mimicking the shape and size of PRINT® mold cavities comprised of greater than 99% ribavirin, of which is greater than 99% polymorph Form II, are formed through an amorphous solid to crystalline solid crystallization process in less than 15 days. In embodiments, the amorphous solid to crystalline solid crystallization process does not include a liquid transition.

The present invention provides method of fabricating a plurality of particles having substantially uniform size and shape and being comprised of greater than 95% ribavirin, of which is greater than 95% polymorph form II, in less than 15 days. More preferably, the present invention provides method of fabricating a plurality of particles having substantially uniform size and shape and being comprised of greater than 95% ribavirin, of which is greater than 99% polymorph form II, in less than 15 days. More preferably, the present invention provides method of fabricating a plurality of particles having substantially uniform size and shape and being comprised of greater than 98% ribavirin, of which is greater than 99% polymorph form II, in less than 15 days.

According to an embodiment of the present invention, a method is provided for retaining crystalline polymorph form of a composition while reducing particulate size by greater than 5 times. According to another embodiment of the present invention, a method is provided for retaining crystalline polymorph form of a composition while reducing particulate size by greater than 15 times. According to an embodiment of the present invention, a method is provided for retaining crystalline polymorph form of a composition while transferring the physical form factor of the composition from random shape and sized particulates to uniform particles with a reduced particulate size of greater than 5 times. According to an embodiment of the present invention, a method is provided for retaining crystalline polymorph form of a composition while transferring the physical form factor of the composition from random shape and sized particulates to uniform particles with a reduced particulate size of greater than 15 times.

### Dose Containment

The fabricated particles described herein may be metered as individual doses, and delivered in a number of ways. Additional aspects of the invention relate to dosage forms and inhalers for delivering metered quantities of the compositions of the present invention. In such aspects, the composition of the present invention is in the form of a dry powder composition deliverable from a dry powder inhaler or as a pressurized liquid propellant suspension formulation delivered from a pressurized metered dose inhaler.

Thus, in one or more embodiments, the invention is directed to a dosage form adapted for administration to a patient by inhalation as a dry powder.

### Unit Dose and Multidose Dry Powder Inhalers (DPIs)

The pharmaceutical composition of the present invention may be contained within a dose container containing a single dose of the pharmaceutical composition. In one or more embodiments, the dose container may be a capsule, for example, the capsule may comprise hydroxypropyl methylcellulose, gelatin or plastic, or the like.

In a still further aspect of the present invention, the invention relates to a dry powdered inhaler which contains one or more pre-metered doses of the compositions of the present invention. The containers may be rupturable, peelable or otherwise openable one-at-a-time and the doses of the dry powder composition may be administered by inhalation on a mouthpiece of the inhalation device, as known in the art.

Thus, compositions of the present invention may be presented in capsules or cartridges (one dose per capsule/cartridge) which are then loaded into an inhalation device, typically by the patient on demand. The device has means to rupture, pierce or otherwise open the capsule so that the dose is able to be entrained into the patient's lung when they inhale at the device mouthpiece. As marketed examples of such devices there may be mentioned ROTAHALER™ of GlaxoSmithKline (described for example in US 4,353,365), the HANDIHALER™ of Boehringer Ingelheim. or the BREEZHALER™ of Novartis, and MONODOSE™ of Plastiape S.p.a. (Osnago (Lecco), Italy).

Multi-dose dry powder forms containing the pharmaceutical composition described herein may take a number of different forms. For instance, the multi-dose may comprise a series of sealed blisters with the composition sealingly contained in a blister pocket, and may be arranged as a disk-shape or an elongated strip. Representative inhalation devices which use such multi-dose forms include devices such as the DISKHALER™, DISKUS™ and ELLIPTA™ inhalers marketed by GlaxoSmithKline. DISKHALER™ is described for example in U.S. Pat. No. 4,627,432 and U.S. Pat. No. 4,811,731. The DISKUS™ inhalation device is, for example, described in U.S. Pat. No. 5,873,360 (GB 2242134A). The ELLIPTA inhaler is described for example in U.S. Pat. No. 8,511,304, U.S. Pat. No. 8,161,968, and U.S. Pat. No. 8,746,242.

Alternatively, compositions of the present invention may be administered via a dry powder reservoir based, meter-in-device dry powder inhaler, wherein the composition of the present invention is provided as a bulk in a reservoir of the inhaler. The inhaler includes a metering mechanism for metering an individual dose of the composition from the reservoir, which is exposed to an inhalation channel, where the metered dose is able to be inhaled by a patient inhaling at a mouthpiece of the device. Exemplary marketed devices of this type are the TURBUHALER™ of AstraZeneca, TWISTHALER™ of Merck and CLICKHALER™ of Innovata.

In addition to delivery from passive devices, compositions of the present invention may be delivered from active devices, which utilize energy not derived from the patient's inspiratory effort to deliver and deagglomerate the dose of the composition.

The dry powder pharmaceutical composition may be delivered solely in the form of the fabricated particles of ribavirin and excipient.

Alternatively, the fabricated particles may be admixed with a pharmaceutically acceptable carrier/diluent, such as lactose or mannitol, with or without further excipients materials, such as lubricants, amino acids, polypeptides, or other excipients noted to have beneficial properties in such pharmaceutically acceptable carrier/diluent formulations, which combined form a finely divided powder.

In one or more embodiments of the present invention, the dry powder compositions of the invention have a moisture content below about 10% by weight water, such as a moisture content of about 9% or below; such as about 9, 8, 7, 6, 5, 4, 3, 2, or 1% or below by weight water. In one or more embodiments of the invention, the dry powder composition has a moisture content below about 1% by weight water.

### Metered Dose Inhalers (MDIs)

It is also considered within the scope of the present invention that the pharmaceutical composition described herein may be formulated in a suitable liquid pressurized liquid propellant, for use in a metered dose inhaler (MDI).

Thus, a further aspect of the invention is an inhaler, as well as a liquid propellant formulation for use therein. Such inhalers may be in the form of a metered dose inhaler (MDI) generally comprising a canister (e.g., an aluminium canister) closed with a valve io (e.g. a metering valve) and fitted to an actuator, provided with a mouthpiece, and filled with a liquid pressurized liquid propellant formulation containing the pharmaceutical compositions as described herein. Examples of suitable devices include metered dose inhalers, such as the Evohaler® (GSK), Modulite® (Chiesi), SkyeFine™ and SkyeDry™ (SkyeP harma).

When formulated for metered dose inhalers, the compositions in accordance with the present invention are formulated as a suspension in a pressurized liquid propellant. In one or more embodiments of the present invention, while the propellant used in the MDI may be CFC-11, and/or CFC-12, it is possible that the propellant be an ozone friendly, non-CFC propellant, such as 1,1,1,2-tetrafluoroethane (HFC 134a), 1,1,1,2,3,3,3-heptafluoro-n-propane (HFC-227), HCFC-22 (difluororchloromethane), or HFA-152 (difluoroethane and isobutene), either alone or in any combination.

Such formulations may be composed solely of propellant and the fabricated particles described herein, or alternatively may also include one or more surfactant materials, such as polyethylene glycol, diethylene glycol monoethyl ether, polyoxyethylene sorbitan monolaurate, polyoxyethylene sorbitan monooleate, propoxylated polyethylene glycol, polyoxyethylene lauryl ether, oleic acid, lecithin or an oligolactic acid derivative e.g. as described in WO94/21229 and WO98/34596, for suspending the composition therein, and may also include agents for solubilising (co-solvents may include, e.g. ethanol), wetting and emulsifying components of the formulation, and/or for lubricating the valve components of the MDI, to improve solubility, or to improve taste.

In one or more embodiments of the invention, the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

The stability of the suspension aerosol formulations according to the invention may be measured by conventional techniques, for example, by measuring flocculation size distribution using a back light scattering instrument or by measuring particle size distribution by cascade impaction, employing a cascade impactor, for example the Next Generation Impactor (NGI), or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above. In the context of his application, the NGI is used unless otherwise indicated.

MDI canisters generally comprise a container capable of withstanding the vapor pressure of the propellant used such as a plastic or plastic-coated glass bottle or preferably a metal can, for example, aluminum or an alloy thereof which may optionally be anodized, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

There is thus provided as a further aspect of the invention a pharmaceutical aerosol formulation comprising an amount of the fabricated particles as previously described and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

In a further embodiment, the invention is directed to a dosage form adapted for administration to a patient by inhalation via a metered dose inhaler.

The high solubility and low lipophilicity of ribavirin are both favorable characteristics in terms of lung delivery of the fabricated particles of the present invention. In some embodiments, the clinical dosing regimen of the compositions of the present invention is io 1-100 mg QD, or 1-50 mg BID, or in some embodiments, 30 mg BID, in other embodiments, 60 mg BID.

The aerosol formulations of the present invention are preferably arranged so that each metered dose, either in dry powder or in a given “puff” from a MDI, contains from 1 mg to 100 mg, or from 3 mg to 75 mg, or from about 5 mg to 50 mg of ribavirin, or from 7.5 mg to 30 mg ribavirin. Administration may be once daily or several times daily, for example 2, 3, 4 or 8 times, giving for example 1, 2 or 3 doses each time. In certain embodiments, the overall daily dose of ribavirin with an aerosol will be within the range from 100 μg to 50 mg, preferably from 750 μg to 3500 μg. The overall daily dose and the metered dose delivered by capsules and cartridges in an inhaler or insufflator will generally be double that delivered with aerosol formulations.

In the case of suspension aerosol formulations, the particle size of the fabricated particles of ribavirin should be such as to permit inhalation of substantially all the drug into the lungs upon administration of the aerosol formulation and will thus be less than 100 μm, desirably less than 20 μm, and in particular in the range of from 1 to 10 μm, such as from 1 to 5 μm, more preferably from 2 to 3 μm.

Ratios of material to material using in the compositions described are to be understood to be weight:weight measures, e.g. RBV:Trehalose: Trileucine (35:55:10) is on w:w:w basis.

In one instance, a ribavirin dose administered to a subject, using a ribavirin composition of RBV: Trehalose: Trileucine (35:55:10), could be 30 mg given BID. This ribavirin dose could be delivered to the subject with a single unit inhaler provided with 4 capsules using a suitable inhaler, such as a Monodose™ or a Rotahaler™. The device design may be optimized to achieve desired product delivery characteristics, for example by reducing the air inlet dimensions to increase velocity, etc. . . . Each capsule would contain 7.5 mg ribavirin and the subject will perform 2 inhalations per capsule.

In another instance, a ribavirin dose administered to a subject using a ribavirin composition of RBV, and could be given 60 mg given BID. This ribavirin dose could be delivered to the subject with a suitable single unit inhaler, such as a Monodose™, provided with two (2) capsule(s). Each capsule would contain 30 mg ribavirin and the subject will perform 1 inhalations per capsule. In one embodiment, the RBV composition would comprise from 95-99.9% w:w of crystalline polymorph Form II RBV (e.g. about RBV:PVOH (95-99.9%: 5-0.1%), e.g. RBV:PVOH (99:1).

### EXAMPLES

The invention is now described in relation to a number of non-limiting examples:

### Particle Fabrication and Analysis

### Example 1A

### Preparation of Dextran/Ribavirin/PVOH Particles

### 1.A.1. Particle Precursor Solution

Prepare an aqueous particle precursor solution according to the following table. Weigh Water for Injection (WFI) into an appropriate sized vessel. Sequentially, with stirring, add PVOH, dextran, and ribavirin. After at least 45 minutes of stirring visually verify that components have dissolved. Once components have dissolved, sterile filter using a 0.22 micron filter. Store at 4° C. until use.

TABLE 5
Ribavirin:Dextran:PVOH

| Component | Weight Percent |
|---|---|
| Water, WFI | 90.5009 |
| Dextran, ~6,000 g/mole | 6.1133 |
| Ribavirin | 3.3250 |
| PVOH, ~6,000 g/mole, 78% hydrolyzed | 0.0608 |
| Total | 100.0000 |

Based on solids, the particle precursor contains the components and amounts detailed in Table 6 below.

TABLE 6
Solids in Stock Solution

| Component | Weight Percent (Solids) |
|---|---|
| Dextran, ~6,000 g/mole | 64.35 |
| Ribavirin | 35.00 |
| PVOH, ~6,000 g/mole, 78% hydrolyzed | 0.64 |

### 1.A.2. Particle Fabrication

a. Deposit the particle precursor solution on a sheet (for example, PET sheet) under ambient temperature (approximately 73° F.) and approximately 65%-75% relative humidity. Dry the particle precursor solution on the sheet to make a thin film containing the particle precursor. In the current embodiments the thin film of the above stock solutions is cast to be between 0.3-0.5 μm thick, more preferably about 0.4 μm thick.
b. The thin film is dried to drive off the water content in the particle precursor solution. In an example, about 95% or more of the water is dried off.
c. Next, the thin film containing the particle precursor on the sheet is mated with a PRINT® mold and passed through a heated nip (rollers) (330+/−10° F.) with pressure at the nip (60+/−5 psi) to form a laminate of web/mold with the particle precursor there between. The PRINT® mold includes the mold cavities, or features, facing the particle precursor in the laminate and upon the laminate passing through the heated nip point, the particle precursor flows into the mold cavities.
d. As the laminated mold leaves the nip roller the particle material in the cavities solidifies and assumes the three-dimensional shape of the mold cavity with a flash layer of the particle material remaining and thereby connecting each mold cavity in crystallization communication.
e. Separately, after annealing, the mold film and web film are separated and the particles are harvested.

### 1.A.3 Harvesting of Particles

Following separation of the mold film and web film, the particles are removed from the mold cavities and remain on the web until removed from the web and collected as a powder.

In the current Example, a 1 μm “pollen” shape was utilized, wherein the 1 μm is the maximum overall lateral dimension of the particle in top-plan view and the particle is between 0.55 μm and 0.60 μm in thickness.

FIG. 1A and FIG. 1B are scanning electron microscope (SEM) images of 35:64:1 w/w ribavirin:dextran:PVOH 1μm pollen PRINT° particles.

### Example 1B

### Preparation of Ribavirin/Trehalose/Trileucine Particles

### 1.B.1. Particle Precursor Solution

Prepare an aqueous particle precursor solution according to the following Table 7. Weigh WFI into an appropriate sized vessel. Sequentially, with stirring, add 10% HCl and trileucine. Stir overnight at ambient temperature to dissolve the trileucine. With stirring, add trehalose dihydrate and ribavirin. After at least 30 minutes of stirring visually verify that components have dissolved. Once components have dissolved, sterile filter using a 0.22 micron filter. Store at 4° C. until use.

TABLE 7
Ribavirin:Trileucine:Trehalose

| Component | Weight Percent |
|---|---|
| Water, WFI | 89.983 |
| 10% HCI | 0.908 |
| Trileucine | 0.913 |
| Trehalose dihydrate | 5.008 |
| Ribavirin | 3.188 |
| Total | 100.000 |

Based on solids, the particle precursor contains the components and amounts detailed in Table 8 below.

TABLE 8
Solids in Stock Solution

| Component | Weight Percent (Solids) |
|---|---|
| Trileucine | 10.0 |
| Trehalose dihydrate | 55.0 |
| Ribavirin | 35.0 |

### 1.B.2. Particle Fabrication

a. Deposit the particle precursor solution on a sheet (for example, PET sheet) under ambient temperature. Dry the particle precursor solution on the sheet to make a thin film containing the particle precursor. In the current embodiments the thin film of the above stock solutions is cast to be between 0.3-0.5 μm thick, more preferably about 0.4 μm thick.
b. The thin film is dried to drive off the water content in the particle precursor solution. In an example, about 95% or more of the water is dried off.
c. Next, the thin film containing the particle precursor on the sheet is mated with a PRINT® mold and passed through a heated nip (rollers) (330+/−10° F.) with pressure at the nip (60+/−5 psi) to form a laminate of web/mold with the particle precursor there between. The PRINT® mold includes the mold cavities, or features, facing the particle precursor in the laminate and upon the laminate passing through the heated nip point, the particle precursor flows into the mold cavities.
d. As the laminated mold leaves the nip roller the particle material in the cavities solidifies and assumes the three-dimensional shape of the mold cavity with a flash layer of the particle material remaining and thereby connecting each mold cavity in crystallization communication.
e. Separately, the mold film and web film are separated and the particles are harvested.

### 1.B.3 Harvesting of Particles

Following separation of the mold film and web film, the particles are removed from the mold cavities and remain on the web until removed from the web and collected as a powder.

In the current Example, a 1 μm “pollen” shape was utilized, wherein the 1 μm is the maximum overall lateral dimension of the particle in top-plan view and the particle is between 0.55 μm and 0.60 μm in thickness.

FIG. 2A and FIG. 2B are SEM images of 35:55:10 ribavirin:trehalose:trileucine 1 μm pollen PRINT° particles.

### Example 1C

### Preparation of Crystalline Controlled Ribavirin/PVOH Particles

### 1.C.1 Particle Precursor Solution

Particles containing ribavirin and PVOH were fabricated using the components in Table 9 below using the following methods.
a. An aqueous PVOH (viscosity=4.8-5.8 mPa·sec at 4% in aqueous at 20° C.) stock solution was prepared.
   1) WFI and PVOH (for example, for 500 g of solution, 499.185 g WFI and 0.8143 g PVOH were weighed) were weighed and combined in an appropriate vessel.
   2) The vessel containing the PVOH and WFI was heated on stir/hot plate set to 90° C. for at least 8 hours with stirring.
   3) The PVOH stock solution was filtered using a 0.2 μm PES filter.
   4) The filtered PVOH stock solution was returned to the 90° C. hot plate and was stirred.
b. RBV was combined with the filtered PVOH stock solution.
   1) The desired amount of filtered PVOH stock solution was weighed (for example, 393.1 g) into an appropriate vessel.
   2) RBV (for example, 64.0 g) was added to the vessel containing the weighed filtered PVOH stock solution.
   3) The vessel containing the RBV and filtered PVOH stock solution was placed onto a stir/hot plate set to 65° C. for at least 20 minutes with stirring.
   4) The RBV/PVOH stock solution was transferred to the fabrication line without further filtration.

TABLE 9
Ribavirin:PVOH

| Component | Stock Solution, g | wt % |
|---|---|---|
| Water, WFI | 392.46 | 85.86 |
| Ribavirin | 64.0 | 14.00 |
| PVOH | 0.64 | 0.14 |
| Total | 457.1 | 100 |

Based on solids, the particle stock solution contained the components and amounts detailed in Table 10 below.

TABLE 10
Solids in Stock Solution

| Component | % based on solids |
|---|---|
| Water, WFI | NA |
| Ribavirin | 99 |
| PVOH | 1 |
| Total | 100 |

### 1.C.2 Particle Fabrication

a. The RBV/PVOH stock solution was deposited on a sheet (for example PET sheet) under ambient temperature about 20 degrees C. and approximately 30% (+/−5%) relative humidity. The RBV/PVOH stock solution was dried on the sheet making a thin film containing RBV and PVOH (particle material). The thin film of the RBV/PVOH stock solution was cast to be between about 500-700 nanometers thick, more preferably between about 550-650 nanometers thick.
b. The thin film was dried to drive off the water. For example, about 95% or more of the water was driven off.
c. The thin film containing the particle material was mated with a PRINT® mold (for example, containing cylinder-shaped cavities about 900 nm in diameter and about 1020 nm in height) and passed through a heated nip (rollers) at approximately 230-250° F. and approximately 60+/−5 psi to form a laminate (PET sheet/particle material/mold). As the laminate passed through the nip, the particle material was urged into the cavities of the mold with a flash layer of the particle material remaining and connecting each mold cavity in crystallization communication.
d. As the laminate exited the nip, the particle material solidified within the mold cavities to form particles mimicking the three-dimensional shape of the mold cavities. Additionally, the particles were affixed with the sheet. The laminate (particles in mold cavities affixed with sheet) may be rolled if desired, for example for storage or further processing of the particles.

### 1.C.3. Annealing

a. If desired, the laminate roll containing particles was annealed. Annealing involves storage of particles and/or laminate rolls containing particles at a specific temperature and relative humidity for specific time duration.
b. Prior to annealing, the laminate roll containing particles may be interleafed with a mesh material to improve exposure of particles to the storage conditions.
c. For example, laminate rolls containing particles were interleafed with a polypropylene mesh (Industrial Netting, catalog XN3234: FDA compliant natural polypropylene mesh with 65% open area, holes approximately 0.054″ X 0.080″, thickness approximately 0.019″).
d. For example, laminate rolls containing particles were stored at 30+/−2° C. and 48+/−5% relative humidity for at least 14 days.

### 1.C.4. Harvesting

a. After fabrication and/or annealing, the particles were removed from the mold cavities. The laminate was separated into mold and sheet layers. The particles remained in contact with the sheet when removed from the mold cavities.
b. The particles were removed from the sheet mechanically by scraping.
c. The particles were sieved using a 250 micrometer mesh sieve.

### 1.C.5 Packaging and Labeling

a. After sieving, the particles were bulk packaged into a labeled, tared Tyvek pouch.
b. Tyvek pouches were placed into foil pouches with desiccant for storage.
c. Bulk packaged material was stored at −20° C. until use.

The 99:1 w/w ribavirin:PVOH (RBV:PVOH) material is made of discreet cylindrical shaped particles as depicted by electron microscopy (FIG. 3A).

### Experimental Characterization And Analysis

Next Generation Impaction Characterization: Ribavirin/Trehalose/Trileucine Particles

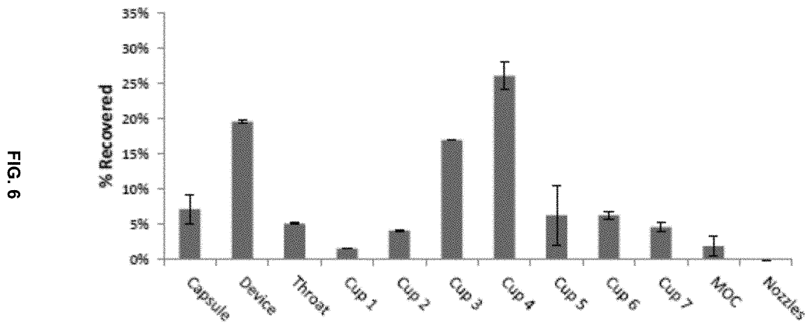

FIG. 6 depicts next generation impaction (NGI) characterization of 35:55:10 ribavirin:trehalose:trileucine 1 μm pollen PRINT® particles. (Testing Conditions: 95 LPM, 4 L test volume; Device used: Plastitape monodose model 8 DPI, 10 mg fill weight (n=2)).

The 35:55:10 w/w ribavirin:trehalose:trileucine (RBV:T:T) material is made of discreet pollen shaped particles as depicted by electron microscopy (FIG. 2A and FIG. 2B), and demonstrates desirable aerosol characteristics including a narrow aerodynamic particle size distribution (GSD<2), and a MMAD within the respirable range (e.g., 1-5 um), and high emitted dose and fine particle fractions.

Table 11 depicts Aerosol Parameters for Ribavirin/Trehalose/Trileucine Particles (35:55:10 ribavirin:trehalose:trileucine (1 μm pollen)).

TABLE 11
Aerosol parameters generated for 35:55:10
ribavirin:trehalose:trileucine

| MMAD in μm | GSD | ED (% rec) | FPF (% ED, <5 μm)* |
|---|---|---|---|
| (STDEV) | (stdev) | (stdev) | (stdev) |
| 1.84 (0.04) | 1.60 (0.01) | 73% (2%) | 88% (1%) |

*Fine particle fraction (FPF) calculation includes Cup 3 and smaller (including the MOC), plus the portion of Cup 2 attributed to particle sizes from 3.51 to 5.0 μm.

The NGI data in Table 11 was generated using a flow rate of 95 L/min with a 2.5 sec actuation for a 4 L induction volume. NGI stage cut points were as follows in Table 12:

TABLE 12
NGI Stage Sizing

| | Cut Diameter | Upper Limit of Range |
|---|---|---|
| Stage | (μm) | in Diameter (μm) |
| 1 | 6.29 | |
| 2 | 3.51 | 6.29 |
| 3 | 2.24 | 3.51 |
| 4 | 1.34 | 2.24 |
| 5 | 0.74 | 1.34 |
| 6 | 0.42 | 0.74 |
| 7 | 0.25 | 0.42 |
| MOC | | 0.25 |

RBV:Trileucine:Trehalose particles showed good maintenance of particle morphology following storage for 1 month under all conditions. Additionally, all chemical, physical, and aerodynamic properties were found to be intact following 1 month storage under nitrogen or dry conditions and the particles showed no crystallization.

### Example 2

### In Vivo Study in Wistar Hannover Rat

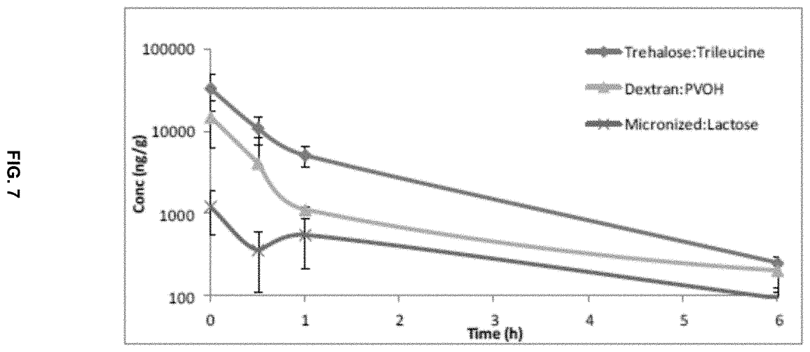

FIG. 7 depicts the comparative ribavirin concentration in Wistar Hannover (WH) rat lung homogenates following a single inhaled dose at 2 mg/kg for ribavirin:trehalose:trileucine (RBV:T:T) and ribavirin:dextran:polyvinyl alcohol (RBV:D:PVOH) PRINT® and micronized lactose solid formulations. An inhaled pharmacokinetic evaluation was performed using highly characterized RBV:T:T and RBV:D:PvOH PRINT® particles (1 μm; pollen shape; MMAD=1.84 μm and 2.29 μm, respectively). Exposures were compared with exposure following dosing of a micronized lactose formulation (MMAD=2.9 μm). RBV particles formulated with approximately 35% loading were delivered using a Wright Dust Feeder (WDF) and ADG inhalation tower to the WH rats at doses of approximately 2 mg/kg for RBV:T:T, RBV:D:PvOH and RBV: Lactose formulations for 15 minutes. The lowest RBV lung exposure as measured by Cmax and AUCo-t was observed with the lactose formulation. Table 14 represents the data summary from a single inhalation dose study for various PRINT® particles. Data was from the Han Wistar rat study following a single inhalation.

TABLE 13
Plasma vs. Lung - Tmax, Cmax and AUC values in animal models

| | | | Nominal Dose | | | Cmax | AUC | |
|---|---|---|---|---|---|---|---|---|
| | | | of ribavirin | Tmax (hr) | | (μg/mL) | (μg*hr/mL) | Source |
| Plasma | Rat | PRINT ® | 2 mg/kg | <0.5 | hr | 0.12-1.25 | 0.76-2.37 | Rat PK Study, |
| | | trehalose:Trileucine | | | | | | n = 6 |
| | | PRINT ® | 2 mg/kg | <0.5 | hr | 0.10-0.88 | 0.68-1.91 | Rat PK Study, |
| | | dextran:PVOH | | | | | | n = 6 |
| | | Lactose | 2 mg/kg | 1 | hr | 0.08-0.28 | 0.52-2.20 | Rat PK Study, |
| | | | | | | | | n = 6 |

| | | | | | | | |
|---|---|---|---|---|---|---|---|
| ELF | Rat | PRINT ® | 2 mg/kg | IAD | 1018-1567 | 325-2843 | Rat PK Study, |
| | | trehalose:trileucine | | | | | n= 3 |
| | | PRINT ® | 2 mg/kg | IAD | 790-1202 | 185-644 | Rat PK Study, |
| | | dextran:PVOH | | | | | n= 3 |
| | | Lactose | 2 mg/kg | IAD | N/A | N/A | Rat PK Study, |
| | | | | | | | n= 3 |
| Lung | Rat | PRINT ® | 2 mg/kg | IAD | 18.7-50.1 | 17.4-32.0 | Rat PK Study, |
| | | trehalose:Trileucine | | | | | n= 3 |
| | | PRINT ® | 2 mg/kg | IAD | 5.0-19.0 | 15.4-26.0 | Rat PK Study, |
| | | dextran:PVOH | | | | | n= 3 |
| | | Lactose | 2 mg/kg | IAD | 0.5-1.8 | 1.8-4.2 | Rat PK Study, |
| | | | | | | | n= 3 |

TABLE 14
Data summary from a single inhaiation dose study for PRINT ® particle

| | | | | | |
|---|---|---|---|---|---|
| | | Fold change in | | | |
| | Lung⁴ | Lung Cmax | Lung | Plasma | Plasma⁴ |
| MMAD | Cₘₐₓ | compared to | AUC(0-t) | Cₘₐₓ | AUC(0-t) |
| (±GSD) | (μg/g)/ | micronized blend | (μg*h/g)/ | (μg/mL)/ | (μg*h/mL)/ |
| μm | (mg/kg) | in lactose | (mg/kg) | (mg/kg) | (mg/kg) |

Fabricated particles of the Invention

| | | | | | | |
|---|---|---|---|---|---|---|
| RBV:PVOH (Crystalline)¹ | 2.1 | 16.4 | 24x | 10.6 | 0.0971 | 0.363 |
| | (2.5) | [[13.96]] | [[19X]] | [[9.06]] | [[0.083]] | [[0.309]] |
| RBV:trehalose:trileucine² | 0.9 | 17.6 | 26x | 13.8 | 0.199 | 0.502 |
| | (3.4)³ | [[18.17]] | [[25X]] | [[14.26]] | [[0.205]] | [[0.517]] |
| | 2.0 | | | | | |
| | (2.2) | | | | | |
| RBV:dextran/PVOH² | 0.7 | 6.77 | 10x | 4.50 | 0.116 | 0.355 |
| | (2.9)³ | [[6.11]] | [[9X]] | [[4.06]] | [[0.105]] | [[0.320]] |
| | 1.7 | | | | | |
| | (2.0) | | | | | |
| RBV:HPMC-AS² | 1.3 | 12.4 | 18x | 9.13 | 0.0665 | 0.293 |
| | (2.6)³ | [[12.16]] | [[17X]] | [[8.92]] | [[0.065]] | [[0.286]] |
| | 1.9 | | | | | |
| | (2.1) | | | | | |
| RBV: Lactose² | 1.1 | 4.99 | 7x | 5.78 | 0.0758 | 0.209 |
| | (3.4)³ | [[7.13]] | [[10X]] | [[8.26]] | [[0.108]] | [[0.296]] |
| | 2.4 | | | | | |
| | (2.1) | | | | | |

Control

| | | | | | | |
|---|---|---|---|---|---|---|
| micronized blend in | 2.9 | 0.683 | 1X | 1.14 | 0.0878 | 0.356 |
| lactose | (2.6) | [[0.718]] | [[1]] | [[1.20]] | [[0.092]] | [[0.374]] |
| Spray Dried RBV | 1.3 | 0.1.14 | 2X | 0.600 | 0.077 | 0.122 |
| | (3.02)⁵ | [[0.686]] | [[1X]] | [[0.363]] | [[0.0467]] | [[0.0737]] |
| | 1.2 | | | | | |
| | (3.5) | | | | | |

¹PRINT ® particle −1 μm × 0.9 μm cylinder
²PRINT ® particle −1 μm pollen
³Large amounts of powder on filter of cascade impactor (<0.25 μm) for pollen shaped particles; second value shows MMAD/GSD with filter value excluded for information.
⁴Dose-normalized Cmax and AUC, and actual doses were gravimetrically measured, except where double brackets border, i.e., [[ ]], indicating analytically determined values. Fold changes in lung dose-normalized Cmax are also calculated accordingly.
⁵Duplicate measurements.

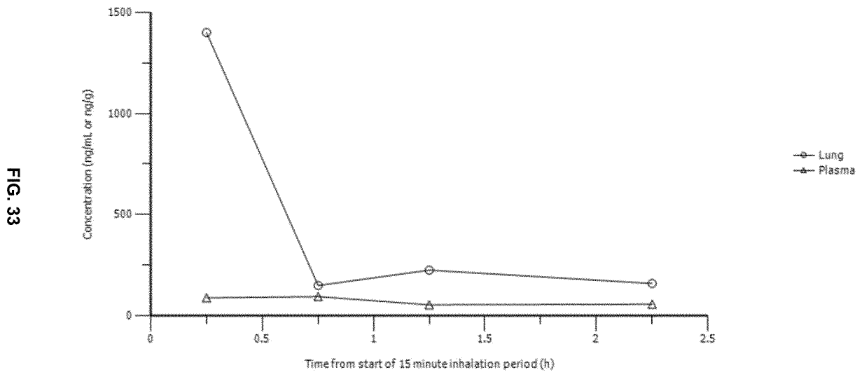

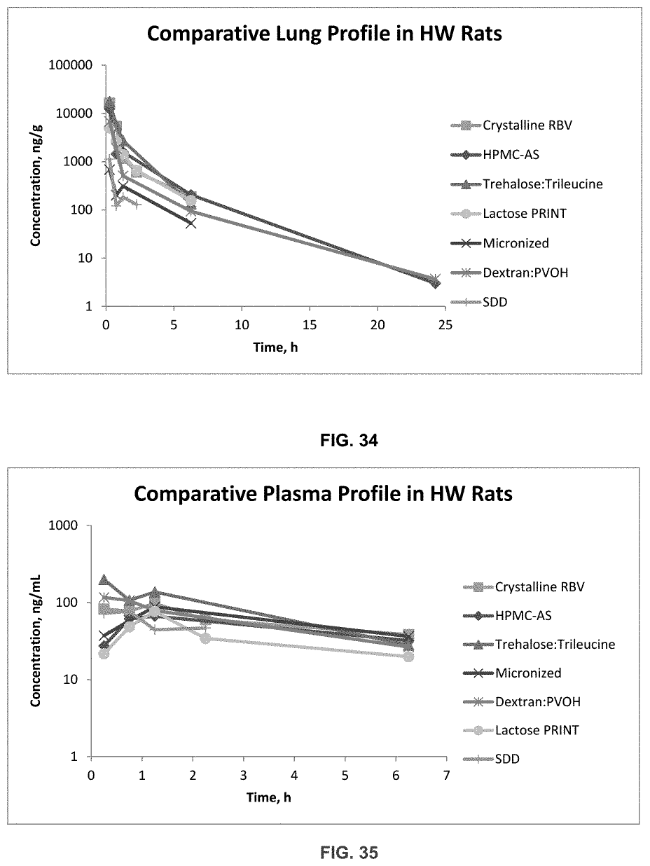

FIGS. 34, 35 and 36 depict the comparative ribavirin concentration in Wistar Hannover (WH) rat lung homogenates and plasmas following a single inhaled dose at 2 mg/kg for PRINT® ribavirin:trehalose:trileucine (Trehalose:Trileucine), PRINT® ribavirin:dextran:polyvinyl alcohol (Dextran:PVOH), PRINT° ribavirin:HPMC-AS (HPMC-AS), PRINT® ribavirin:PVOH (crystalline RBV), PRINT® ribavirin:lactose (Lactose PRINT) and spray dried dispersion ribavirin (SDD) formulations and micronized ribavirin:lactose (Micronized) solid formulations. Formulations were delivered using a Wright Dust Feeder (WDF) or a capsule based aerosol generator (CBAG), and ADG inhalation tower to the WH rats at doses of approximately 2 mg/kg for 15 minutes. The lowest RBV lung exposure as measured by Cₘₐₓ and AUC₀₋ₜ were observed with the lactose and spray dried dispersion formulations.

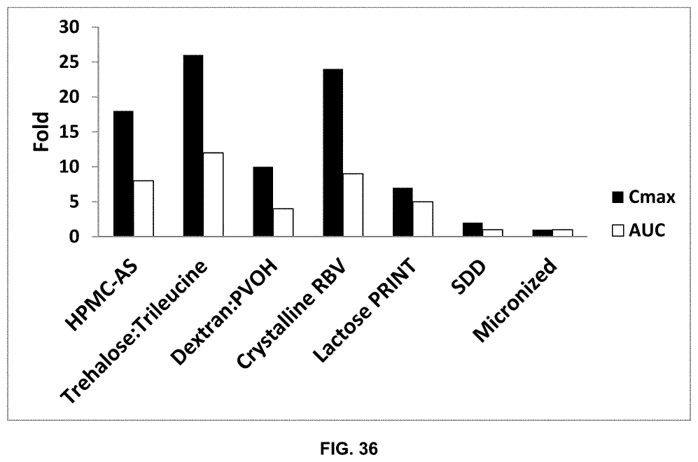

Data from Table 14 are represented in FIGS. 34, 35 and 36. Table 14 and FIG. 36 demonstrate RBV PRINT® formulations can deliver a lung Cₘₐₓ about 7-26 fold higher than micronized conventional RBV in a blend in lactose and spray dried dispersion formulations. This translates to a direct advantage for dosing the patient. For example, a 60 mg dose or ribavirin can be administered by inhaling two (2) capsules of 99% ribavirin in crystalline PRINT® particles compared to approximately 1,000 capsules of 5% ribavirin micronized blend in lactose, to theoretically achieve the same Cmax. (Statement based on calculations that 60 mg crystalline=2×30mg capsules; 60 mg of ribavirin at 5% blend in lactose =1,200 mg; 1,200 mg×26 (fold higher)=31,200 mg; 31,200 mg/30 mg per capsule=1040 capsules). As would be appreciated, 31.2 g of inhaled material would not be a practical inhaled therapy.

### Example 3

Table 15 depicts the toxicokinetics of ribavirin in the Wistar Han rat following snout-only inhalation of ribavirin during a single dose pharmacokinetic study. Briefly, ribavirin concentration analysis were rat plasma and lung homogenate (supernatant) samples analyzed for ribavirin using an analytical method based on protein precipitation followed by HPLC-MS/MS analysis. The lower limit of quantification (LLQ) was 20 ng/mL using a 25 μL aliquot of rat plasma or lung homogenate with a higher limit of quantification (HLQ) of 20000 ng/mL. Plasma samples were analyzed against a plasma calibration line. Lung homogenate (supernatant) samples were analyzed against a lung homogenate (supernatant) calibration line.

TABLE 15
Data summary from a snout-only inhalation of ribavirin
during a single dose pharmacokinetic study for PRINT ®
crystalline and lactose blended particles
Toxicokinetic Parameters:

| | Male | | | |
|---|---|---|---|---|
| | Estimated Inhaled Dose of | | | |
| | Ribavirin (mg/kg) | | | |
| | 1.72 mg/kg (PRINT ® | | 3.89 mg/kg (PRINT ® | |
| | Crystalline Ribavirin) | | Ribavirin with Lactose) | |
| Parameter | Plasma | Lung | Plasma | Lung |
| AUC₀₋ₜ | 624 | 18300 | 814 | 22700 |
| (ng.h/mL or g) | | | | |
| Cₘₐₓ | 167 | 28200 | 298 | 19600 |
| (ng/mL or g) | | | | |
| Tₘₐₓ | 1.25 | 0.25 | 1.25 | 0.25 |
| (h) | | | | |

Table 16 depicts plasma concentrations of ribavirin at each time point following io snout-only inhalation administration of PRINT® crystalline ribavirin at an estimated inhaled dose of 1.72 mg/kg to male rats.

TABLE 16
Data summary of plasma concentrations of ribavirin following snout-only
inhalation administration of PRINT ® crystalline ribavirin

| | | Plasma | Mean Plasma |
|---|---|---|---|
| Time ^{a} | | concentration | concentration |
| (h) | Rat ID | (ng/mL) | (ng/mL) |
| 0.25 | 1.1 | 264 | 141 |
| | 1.2 | 61.9 | |
| | 1.3 | 97.9 | |
| 0.75 | 2.1 | 141 | 133 |
| | 2.2 | 134 | |
| | 2.3 | 125 | |
| 1.25 | 3.1 | 127 | 167 |
| | 3.2 | 256 | |
| | 3.3 | 117 | |
| 2.25 | 4.1 | 66.3 | 103 |
| | 4.2 | 124 | |
| | 4.3 | 119 | |
| 6.25 | 5.1 | 63.2 | 65.6 |
| | 5.2 | 102 | |
| | 5.3 | 31.3 | |
| 24.25 | 6.1 | NQ | NQ |
| | 6.2 | NQ | |
| | 6.3 | NQ | |

^{a} Times are from the start of the 15 minute inhalation period.
NQ—Not Quantifiable

Table 17 depicts plasma concentrations of ribavirin at each time point following snout-only inhalation administration of PRINT® ribavirin with lactose at an estimated inhaled dose of 3.89 mg/kg to male rats.

TABLE 17
Data summary of plasma concentrations of ribavirin
following snout-only inhalation administration
of PRINT ® ribavirin with Lactose

| | | Plasma | Mean Plasma |
|---|---|---|---|
| Time ^{a} | | concentration | concentration |
| (h) | Rat ID | (ng/mL) | (ng/mL) |
| 0.25 | 1.1 | 130 | 83.9 |
| | 1.2 | 84.8 | |
| | 1.3 | 36.6 | |
| 0.75 | 2.1 | 119 | 188 |
| | 2.2 | 108 | |
| | 2.3 | 336 | |
| 1.25 | 3.1 | 358 | 298 |
| | 3.2 | 363 | |
| | 3.2 | 174 | |
| 2.25 | 4.1 | 131 | 133 |
| | 4.2 | 82.9 | |
| | 4.3 | 187 | |
| 6.25 | 5.1 | 83.0 | 77.2 |
| | 5.2 | 87.1 | |
| | 5.3 | 61.4 | |
| 24.25 | 6.1 | NQ | NQ |
| | 6.2 | NQ | |
| | 6.3 | NQ | |

^{a} Times are from the start of the 15 minute inhalation period.
NQ—Not Quantifiable

Table 18 depicts lung concentrations at each time point following snout-only inhalation administration of PRINT® crystalline ribavirin at an estimated inhaled dose of 1.72 mg/kg to male rats.

TABLE 18
Data summary of lung concentrations of ribavirin
following snout-only inhalation administration
of PRINT ® crystalline ribavirin

| | | Lung | Mean lung |
|---|---|---|---|
| Time ^{a} | | concentration | concentration |
| (h) | Rat ID | (ng/g) | (ng/g) |
| 0.25 | 1.1 | 40400 | 28200 |
| | 1.2 | 16800 | |
| | 1.3 | 27300 | |
| 0.75 | 2.1 | 2810 | 9240 |
| | 2.2 | 6560 | |
| | 2.3 | 18400 | |
| 1.25 | 3.1 | 1330 | 2020 |
| | 3.2 | 3800 | |
| | 3.2 | 940 | |
| 2.25 | 4.1 | 1130 | 1050 |
| | 4.2 | 1000 | |
| | 4.3 | 1000 | |
| 6.25 | 5.1 | 524 | 320 |
| | 5.2 | 330 | |
| | 5.3 | 106 | |
| 24.25 | 6.1 | NQ | NQ |
| | 6.2 | NQ | |
| | 6.3 | NQ | |

^{a} Times are from the start of the 15 minute inhalation period.
NQ—Not Quantifiable

Table 19 depicts lung concentrations at each time point following snout-only inhalation administration of PRINT® ribavirin with lactose at an estimated inhaled dose io of 3.89 mg/kg to male rats.

TABLE 19
Data summary of lung concentrations of ribavirin
following snout-only inhalation administration
of PRINT ® ribavirin with Lactose

| | | Lung | Mean lung |
|---|---|---|---|
| Time ^{a} | | concentration | concentration |
| (h) | Rat ID | (ng/g) | (ng/g) |
| 0.25 | 1.1 | 14400 | 19600 |
| | 1.2 | 40000 | |
| | 1.3 | 4320 | |
| 0.75 | 2.1 | 5600 | 10400 |
| | 2.2 | 5520 | |
| | 2.3 | 20100 | |
| 1.25 | 3.1 | 5560 | 5410 |
| | 3.2 | 3100 | |
| | 3.2 | 7560 | |
| 2.25 | 4.1 | 1330 | 2550 |
| | 4.2 | 1630 | |
| | 4.3 | 4680 | |
| 6.25 | 5.1 | 788 | 609 |
| | 5.2 | 488 | |
| | 5.3 | 552 | |
| 24.25 | 6.1 | NQ | NQ |
| | 6.2 | NQ | |
| | 6.3 | NQ | |

^{a} Times are from the start of the 15 minute inhalation period.
NQ—Not Quantifiable

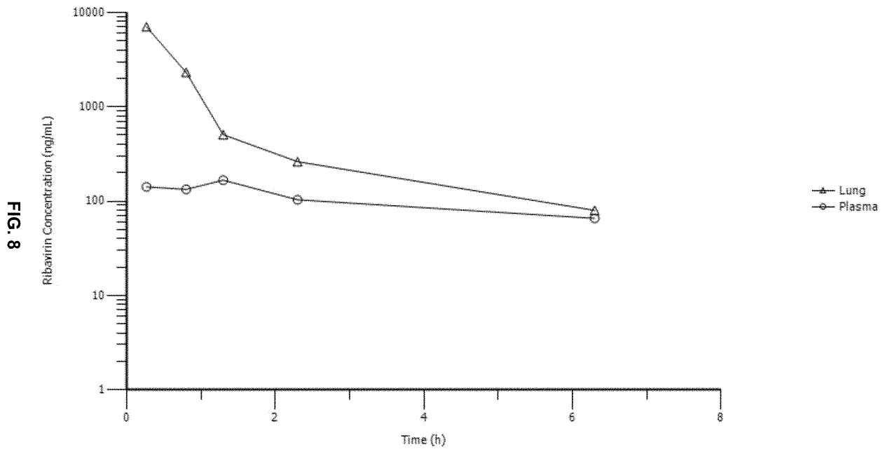

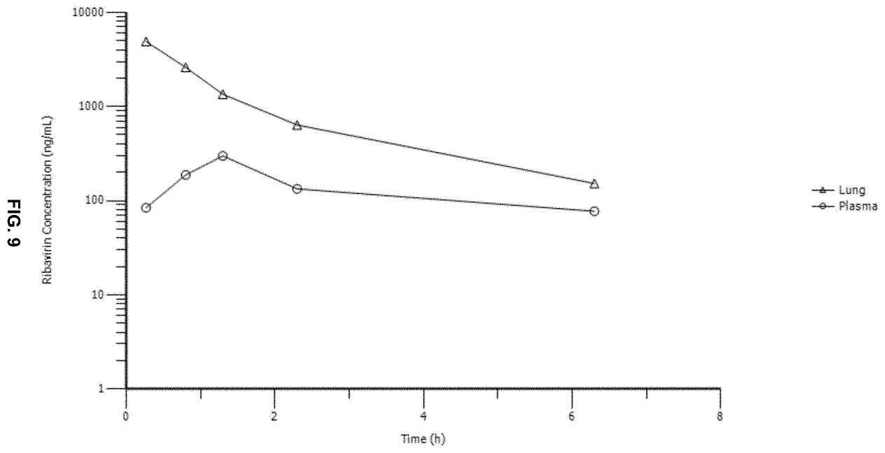

FIG. 8 shows composite mean plasma and lung concentrations of ribavirin following snout-only inhalation of PRINT® crystalline ribavirin at an estimated inhaled dose of 1.72 mg/kg to male rats. FIG. 9 shows composite mean plasma and lung concentrations of ribavirin following snout-only inhalation of PRINT® ribavirin with io lactose at an estimated inhaled dose of 3.93 mg/kg to male rats.

### Example 4

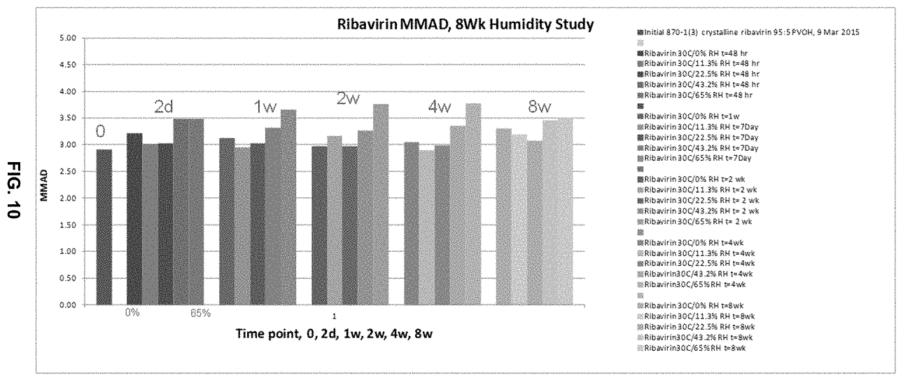

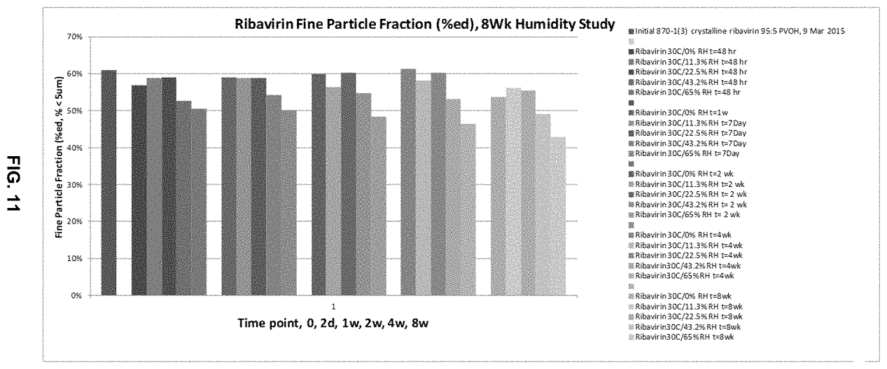

FIG. 10 depicts the MMAD of crystalline ribavirin PRINT® particles (RBV:PVOH (95:5)) stored at 30° C./0% RH, 30° C./11.3% RH, 30° C./22.5% RH, 30° C./43.2% RH, and 30° C./65% RH. FIG. 11 depicts the fine particle fraction of crystalline ribavirin PRINT® particles (RBV:PVOH (95:5)) stored at 30° C./0% RH, 30° C./11.3% RH, 30° C./22.5% RH, 30° C./43.2% RH, and 30° C./65% RH. Testing was conducted at various time intervals over a period of 8 weeks.

### Example 5

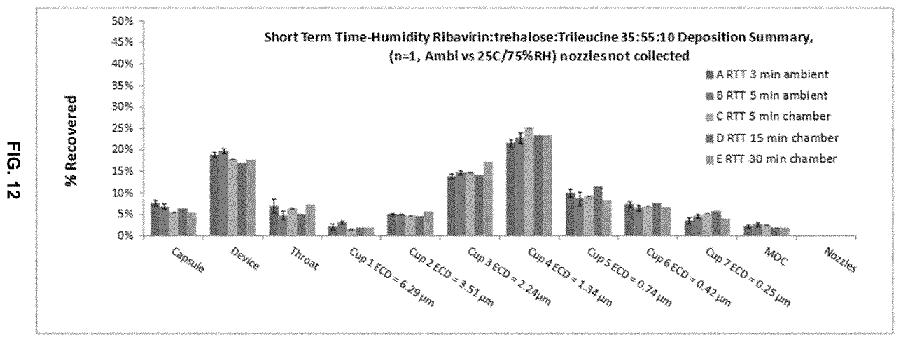

FIG. 12 depicts the deposition summary for Ribavirin:Trehalose:Trileucine (35:55:10) PRINT® particles stored in an ambient environment compared to a storage in a stability chamber. Testing was conducted at various time intervals up to 30 minutes.

### Example 6

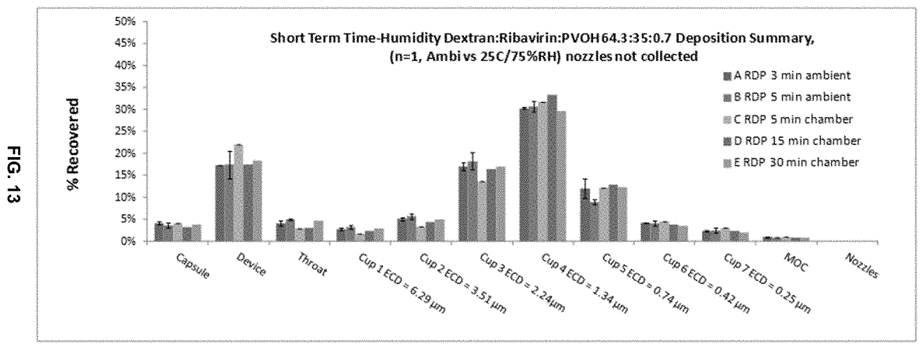

FIG. 13 depicts the deposition summary for Dextran:Ribavirin:PVOH (64.3:35:0.7) PRINT® particles stored in an ambient environment compared to storage in a stability chamber at 25° C./75% relative humidity. Testing was conducted at various time intervals up to 30 minutes.

### Example 7

Table 20 depicts the 1 month in vial stability of RBV:PVOH (97:3) (crystalline) when stored under various conditions.

TABLE 20
Data summary of 1 month in vial Stability of crystalline
RBV (RBV:PVOH (97:3) when exposed stored under various

| | | | | | | | | |
|---|---|---|---|---|---|---|---|---|
| | | | Total | | | | | |
| | | Content | Impurities | MMAD | | | | |
| sample | condition | % RBV | Area % | (μm) | GSD | ED(% rec) | FPF(% ED) | mDSC |

initial

| | | | | | | | | |
|---|---|---|---|---|---|---|---|---|
| 1 | Initial | 94.5 | ND | 1.89 (0.17) | 1.90(0.11) | 73%(1%) | 81%(5%) | Crystalline |
| | | | | | | | | Form II |

1 month

| | | | | | | | | |
|---|---|---|---|---|---|---|---|---|
| 1(2) | −20° C. | 94.1 | 0.09 | 1.88 | 1.87 | 70% | 82% | Crystalline |
| | | | | | | | | Form II |
| 2(2) | 25° C./60% | 94.3 | 0.09 | 1.97 | 1.89 | 74% | 80% | Crystalline |
| | RH closed | | | | | | | Form II |
| 3(2) | 25° C./60% | 93.5 | 0/08 | 2.37 | 2.04 | 78% | 71% | Crystalline |
| | RH open | | | | | | | Form II |
| 4(2) | 40° C./amb | 95.6 | 0.08 | 2.15 | 1.91 | 75% | 76% | Crystalline |
| | RH closed | | | | | | | Form II |

Table 21 depicts the 2 month in vial stability of crystalline RBV (97:3 RBV:PVOH) when stored under various conditions.

TABLE 21
Data summary of 2 month in vial Stability of crystalline
RBV (RBV:PVOH(97:3)) when stored under various conditions

| | | | | | | | | |
|---|---|---|---|---|---|---|---|---|
| | | Content % | Area % | MMAD | | | | |
| sample | condition | RBV | Parent | (μm) | GSD | ED(% rec) | FPF(% ED) | mDSC |

initial

| | | | | | | | | |
|---|---|---|---|---|---|---|---|---|
| 1 | Initial | TBD | TBD | 1.89 (0.17) | 1.90 (0.11) | 73% (1%) | 81% (5%) | Crystalline |

1 month

| | | | | | | | | |
|---|---|---|---|---|---|---|---|---|
| 1(2) | −20° C. | 94.1 | 99.9 | 1.88 | 1.87 | 70% | 82% | Crystalline |
| | | | | | | | | Form II |
| 2(2) | 25° C./60% | 94.3 | 99.9 | 1.97 | 1.89 | 74% | 80% | Crystalline |
| | RH closed | | | | | | | Form II |
| 3(2) | 25° C./60% | 93.5 | 99.9 | 2.37 | 2.04 | 78% | 71% | Crystalline |
| | RH open | | | | | | | Form II |
| 4(2) | 40° C./amb | 95.6 | 99.9 | 2.15 | 1.91 | 75% | 76% | Crystalline |
| | RH closed | | | | | | | Form II |

2 month

| | | | | | | | | |
|---|---|---|---|---|---|---|---|---|
| 1(2) | −20° C. | 93.4 | 99.9 | 1.77 (0.05) | 1.88 (0.01) | 82% (5%) | 86%(0%) | ND |
| 2(2) | 25° C./60% | 94.6 | 99.9 | 1.74 (0.01) | 1.92 (0.06) | 77% (6%) | 84%(1%) | ND |
| | RH closed | | | | | | | |
| 3(2) | 25° C./60% | 97.1 | 99.8 | TBD | TBD | TBD | TBD | ND |
| | RH open | | | | | | | |
| 4(2) | 40° C./amb | 94.0 | 99.8 | TBD | TBD | TBD | TBD | ND |
| | RH closed | | | | | | | |

Table 22 depicts the NGI data for bulk samples of crystalline RBV (RBV:PVOH (97:3)) at 6 months stored in a closed vial with −20° C. room air, in a closed vial with 25° C./60% air, in an open vial continuously exposed 25° C./60% RH air and in a closed vial with 40° C. air.

TABLE 22
Data for NGI data for crystalline RBV (RBV:PVOH (97:3))

| | | | Ribavirin | | | | | | |
|---|---|---|---|---|---|---|---|---|---|
| | | Ribavirin | LC Peak | MMAD | | ED | FPF | Morphology | Polymorph |
| sample | | content % | Purity % | (μm) | GSD | (% rec)** | (% ED) | by SEM | by DSC |
| 1 (2) −20° C. | avg | 96.2% | 99.49% | 1.55 | 1.90 | 100% | 88% | Good | Form II |
| | st dev | 2.0% | 0.61% | 0.03 | 0.00 | 0% | 0% | | |
| 2(2) | avg | 93.5% | 99.92% | 1.60 | 1.99 | 100% | 87% | Good | Form II |
| 25° C./60% | st dev | 4.5% | 0.00% | 0.14 | 0.02 | 0% | 2% | | |
| RH closed | | | | | | | | | |
| 3(2) | avg | 99.9% | 99.92% | 2.20 | 1.90 | 100% | 75% | Good | Form II |
| 25° C./60% | st dev | 1.4% | 0.00% | 0.01 | 0.01 | 0% | 1% | | |
| RH closed | | | | | | | | | |
| (4(2) 40° C. | avg | 95.8% | 99.90% | 1.72 | 1.97 | 100% | 85% | Good | Form II |
| | st dev | 1.5 | 0.00% | 0.04 | 0.01 | 0% | 1% | | |

### Example 8

Table 23 depicts the NGI data for crystalline RBV (RBV:PVOH (99:1)) at 2 months stored in a closed vial with -20° C. room air, in a closed vial with 25° C./60% air, in an open vial continuously exposed 25° C./60% RH air and in a closed vial with 40° C. air.

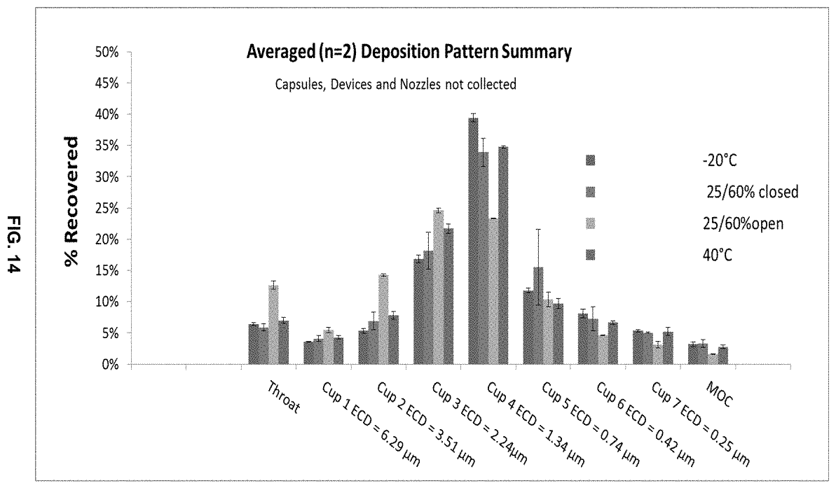

FIG. 14 depicts the NGI deposition pattern for crystalline RBV (RBV:PVOH (97:3)) at 6 months stored in a closed vial with -20° C. room air, in a closed vial with 25° C./60% air, in an open vial continuously exposed 25° C./60% RH air and in a closed vial with 40° C. air.

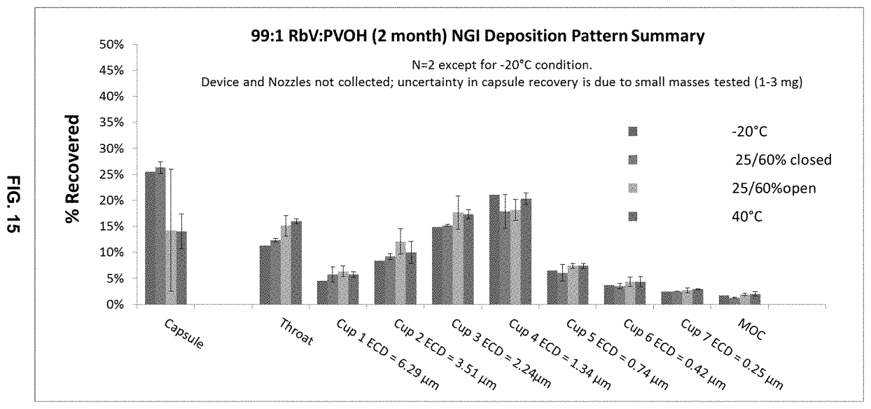

FIG. 15 depicts the deposition pattern for crystalline RBV (RBV:PVOH (99:1)) at 2 months stored in a closed vial with −20° C. room air, in a closed vial with 25° C./60% air, in an open vial continuously exposed 25° C./60% RH air and in a closed vial with 40° C. air.

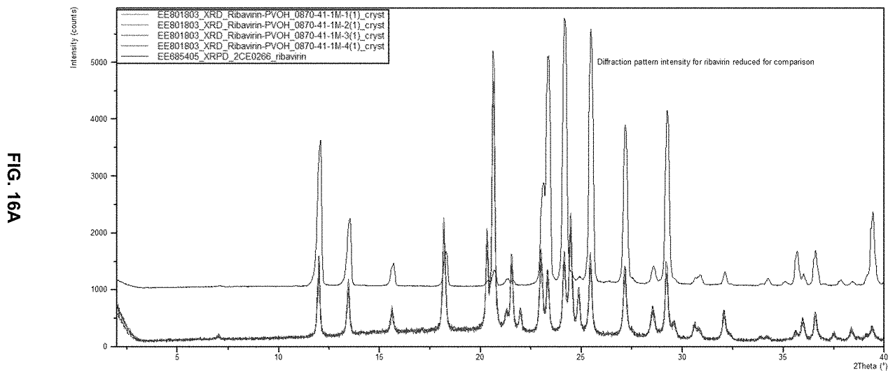

FIG. 16A depicts XRPD data for crystalline ribavirin PRINT particles listed in Table 20. FIG. 16B depicts DSC data for crystalline ribavirin PRINT particles listed in Table 20.

TABLE 23
NGI data for crystalline RBV (99:1 RBV:PVOH)

| | | | Ribavirin | | | | | | |
|---|---|---|---|---|---|---|---|---|---|
| | | Ribavirin | LC Peak | MMAD | | ED | FPF | Morphology | Polymorph |
| sample | | content % | Purity % | (μm) | GSD | (% rec)** | (% ED) | by SEM | by DSC |
| 1 −20° C. | avg | 102.44% | 99.89% | 2.18 | 2.67 | 75% | 74% | Good | Form II |
| | st dev | 1.1% | 0.00 | ** | ** | ** | ** | | |
| 2 | avg | 100.80% | 99.89% | 2.45 | 2.86 | 74% | 70% | Good | Form II |
| 25° C./60% | st dev | 2.4% | 0.00 | 0.07 | 0.21 | 1% | 2% | | |
| RH closed | | | | | | | | | |
| 3(2) | avg | 84.23% | 99.89% | 2.56 | 3.06 | 86% | 68% | Good | Form II |
| 25° C./60% | st dev | 25.6% | 0.01 | 0.21 | 0.19 | 12% | 1% | | |
| RH closed | | | | | | | | | |
| (4(2) 40° C. | avg | 96.96% | 99.90% | 2.32 | 2.88 | 86% | 69% | Good | Form II |
| | st dev | 0.6% | 0.00 | 0.21 | 0.01 | 3% | 1% | | |

### Example 9

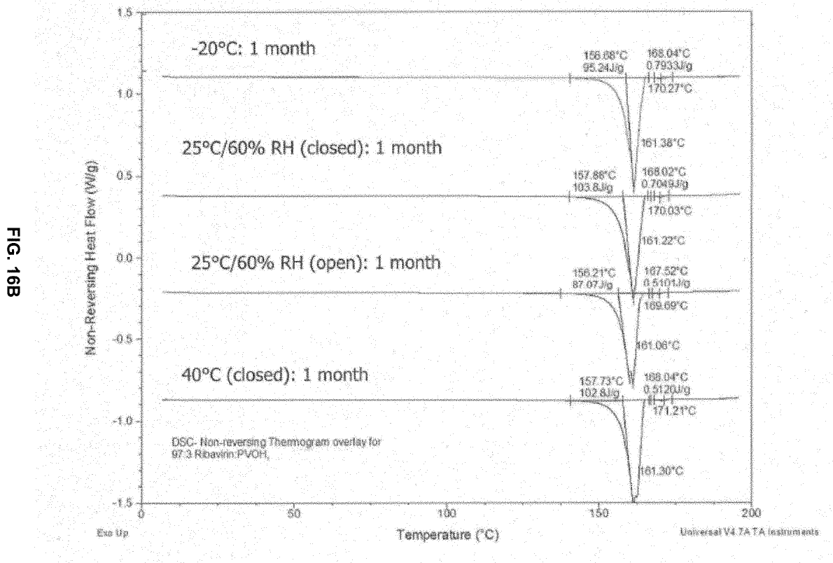

FIG. 17 depicts the ribavirin dissolution profiles for input ribavirin material, Ribavirin:Lactose:PVOH (35:64:1), Ribavirin:Dextran:PVOH (35:64:1), crystalline

Ribavirin:PVOH (97:3) and Ribavirin:Trehalose/Trileucine (35:65) formulations.

### Example 10

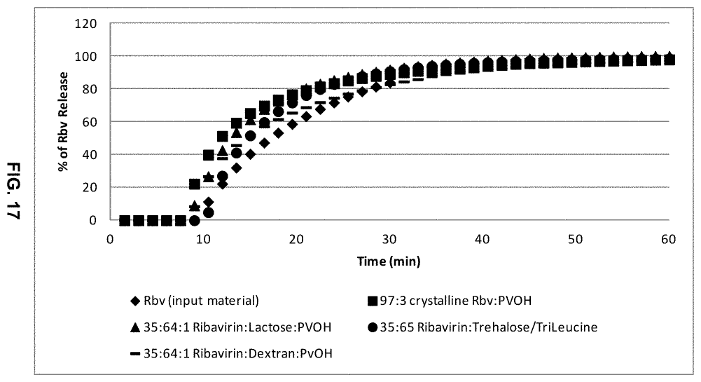

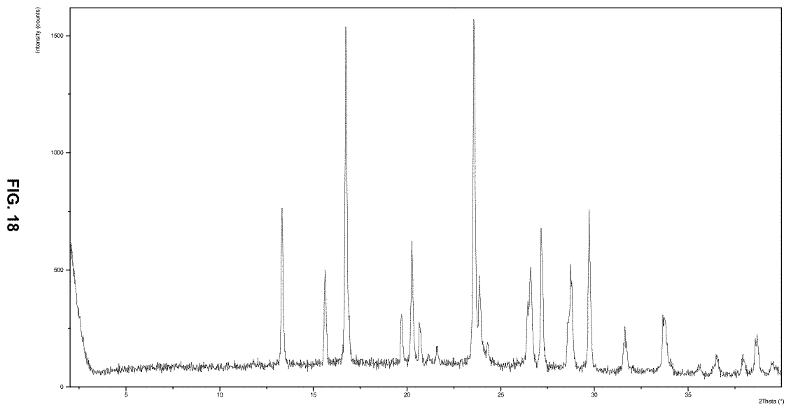

Two polymorphic forms of RBV have been identified, with the Form II polymorph being more stable. Polymorphic forms of RBV may be identified by X-Ray Powder Diffraction (XRPD). The XRPD plot for RBV polymorphic Form I.is depicted in FIG. 18. The XRPD plot for RBV polymorphic Form II is depicted in FIG. 19. X-Ray Powder Diffraction (XRPD) data were acquired on a PANalytical powder diffractometer using an X'Celerator detector. The acquisition conditions were: radiation: Cu Kα, generator tension: 40 kV, generator current: 40-45 mA, start angle: 2.0° 2θ, end angle: 40.0° 2θ, step size: 0.0167° 2θ, time per step: 31.75 seconds. The sample was prepared using front filled approach (A low volume recessed silicon well plate was employed). Characteristic XRPD angles that identify Form I and Form II are recorded in Table 24. The peaks highlighted (bold/*) in Table 24 are the most representative peaks for each polymorphic form. The peaks have been selected to distinguish between the two solid state forms. The margin of error is approximately ±0.15° 2θ for each of the peak assignments. Peak intensities may vary from sample to sample due to preferred orientation. Peak positions were measured using Highscore software.

TABLE 24
Characteristic XRPD peak positions and d-spacings
for Form I and Form II RBV polymorphs

| | |
|---|---|
| RBV Form I | RBV Form II |

| 2θ/° | d-spacings/Å | 2θ/° | d-spacings/Å |
|---|---|---|---|
| 13.3 | 6.6 | 7.1 | 12.5 |
| 15.6 | 5.7 | 12.0* | 7.4 |
| 16.7* | 5.3 | 13.5 | 6.6 |
| 19.7* | 4.5 | 15.6 | 5.7 |
| 20.2 | 4.4 | 18.3* | 4.9 |
| 20.7 | 4.3 | 20.3 | 4.4 |
| 23.6* | 3.8 | 20.7 | 4.3 |
| 23.9 | 3.7 | 21.3 | 4.2 |
| 26.6* | 3.4 | 21.6 | 4.1 |
| 27.2 | 3.3 | 22.0 | 4.0 |
| 28.8 | 3.1 | 23.0* | 3.9 |
| 29.7 | 3.0 | 23.3 | 3.8 |
| 31.6 | 2.8 | 24.1 | 3.7 |
| 33.7 | 2.7 | 24.5 | 3.6 |
| 36.5 | 2.5 | 24.9 | 3.6 |
| 38.0 | 2.4 | 25.4* | 3.5 |
| 38.6 | 2.3 | 27.2 | 3.3 |
| | | 27.5 | 3.2 |
| | | 28.5 | 3.1 |
| | | 29.2 | 3.1 |
| | | 30.6 | 2.9 |
| | | 30.8 | 2.9 |
| | | 32.1 | 2.8 |
| | | 34.2 | 2.6 |
| | | 35.1 | 2.6 |
| | | 35.6 | 2.5 |
| | | 36.0 | 2.5 |
| | | 36.5 | 2.5 |
| | | 37.8 | 2.4 |
| | | 38.4 | 2.3 |
| | | 39.4 | 2.3 |

Thus the most representative peaks for characterizing RBV polymorphic Form I are at about 16.7, 19.7, 23.6 and 26.6 degrees Theta, whereas the most representative peaks for characterizing RBV polymorphic Form II are at about 12.0, 18.3, 23.0 and 25.4 degrees Theta.

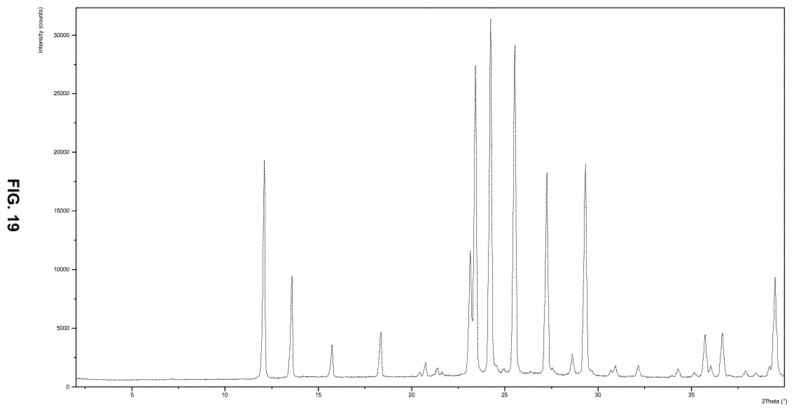

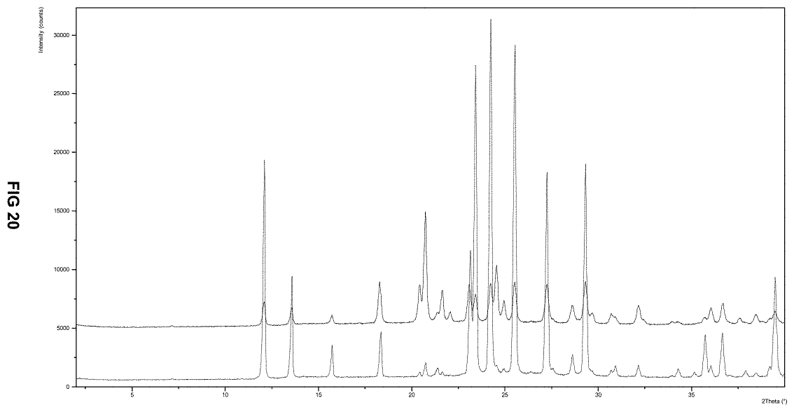

Solid state stability data of the Ribavirin:PVOH (99:1) 0.9×1.0μm Crystalline PRINT particles is depicted in FIG. 20 and FIG. 21. The depicted data demonstrates the solid state stability of 99:1 Ribavirin PVOH 0.9×1.0 μm Crystalline PRINT particles by XRPD.

FIG. 20 shows the XRPD patterns collected at the initial testing. FIG. 20 depicts the XRPD of Ribavirin PRINT particles at the initial time point, and the RBV Form II reference diffraction pattern. The lower pattern (from X-axis) shows the RBV Form II Reference, while the upper pattern shows Initial 99:1 Ribavirin PVOH 0.9×1.0μm Crystalline PRINT particles.

FIG. 21 depicts four XRPD patterns; three Ribavirin PRINT particles at the 6 month timepoint stored at different conditions and the Form II reference diffraction pattern. The pattern closest to and immediately above the x-axis (first pattern) depicts the RBV Form II reference. The pattern immediately above the first pattern (second pattern) depicts 99:1 Ribavirin PVOH 0.9×1.0μm Crystalline PRINT particles stored at 5° C./Amb. The pattern immediately above the second pattern (third pattern) depicts 99:1 Ribavirin PVOH 0.9×1.0μm Crystalline PRINT particles stored at 30° C./65% RH. The pattern immediately above the third pattern (fourth pattern) depicts 99:1 Ribavirin PVOH 0.9×1.0μm Crystalline PRINT particles stored at 40° C./not more than 25% RH. The XRPD patterns of FIG. 21 demonstrate that the diffraction patterns of the 99:1 Ribavirin PVOH 0.9×1.0μm Crystalline PRINT particles at the 6 month timepoint remain concordant with Form II diffraction pattern, indicating no change of solid state form compared to the initial timepoint.

### Example 11

Aerodynamic Particle Size Distribution (APSD) has all been generated by Next Generation Impaction (NGI) equipment utilizing no preseparator and a Throat fitted with rubber integrated mouthpieces. Single capsule APSD determinations of 30.3 mg nominal capsule fill weight using the Monodose RS01 device at a flow rate of 60 litres per minute (L/min) has been provided.

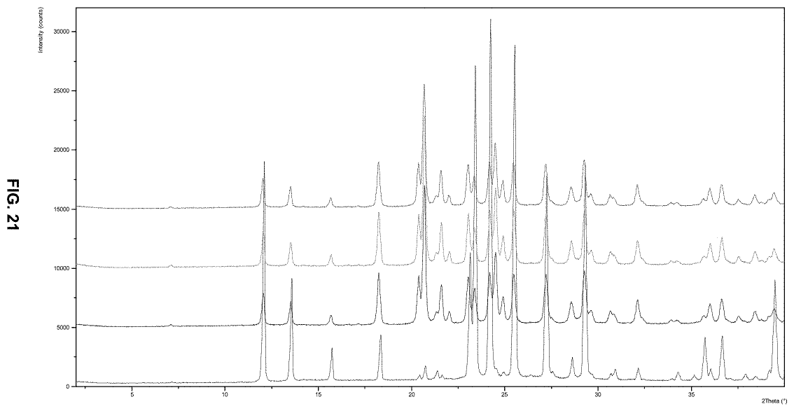

Stability data has been provided in FIGS. 22-26 to show effects of time and environmental conditions on the aerosol performance of the RBV product (as 30.3mg capsules of crystalline 99:1 RBV:PVOH, 0.9×1μm cylinder Liquidia PRINT particles) up to day 28 (DY28). Different pack types have been evaluated on stability:
No Overwrap (Naked)—Capsules stored in a plastic bottle with no further protection
Overwrapped (OW)—Capsules stored in a plastic bottle inside a sealed foil laminate overwrap pouch
Overwrapped & Desiccated (OW+D)—Capsules stored in a plastic bottle inside a sealed foil laminate overwrap pouch. The bottle contains a silica gel desiccant sachet with the capsules and a silica gel desiccant canister is held between the bottle and the foil overwrap. Silica gel desiccants typically yield relative humidity (RH) conditions of <15% RH.
Conditions tested: 5° C./Amb RH, 25° C./60% RH, 40° C./75% RH, 50° C./Amb RH, C-5:40° C. (the latter being a cycling condition between −5° C. and 40° C.). 50° C./Amb RH and C-5:40° C. stored samples are not usually tested for routine stability and have only been included in this study to justify conditions for distribution/shipping and any excursions that may occur.

All data is expressed as % Label Claim (%LC) to show the relative distributions in each stage or summary group.

The 30.3 mg nominal fill weight is equivalent to 30mg RBV for capsules filled with 99:1 RBV:PVOH formulation. Emitted Dose has been taken from NGI Totals (Sum of stages Throat, Stages 1-7, MOC, External Filter).

FIG. 22 depicts a Variability Plot showing Capsule & Device Deposition, Fine Particle Mass and Emitted Dose by NGI Stability data up to DY28 for 30mg RBV Capsules using the Monodose RS01 Device at 60 L/min for 4 seconds (utilizing the crystalline 99:1 RBV:PVOH, 0.9×1 μm cylinder Liquidia PRINT formulation). FIG. 22 shows all data generated at the 5° C./Amb RH, 25° C./60% RH, 40° C./75% RH conditions. Note that only 5° C./Amb RH, 25° C./60% RH is available for Naked storage. The plot shows that across the different pack types and/or conditions up to DY28, there are no significant differences shown for either Capsule deposition, Device deposition, Fine Particle Mass (FPM) or Emitted Dose (as NGI Totals).

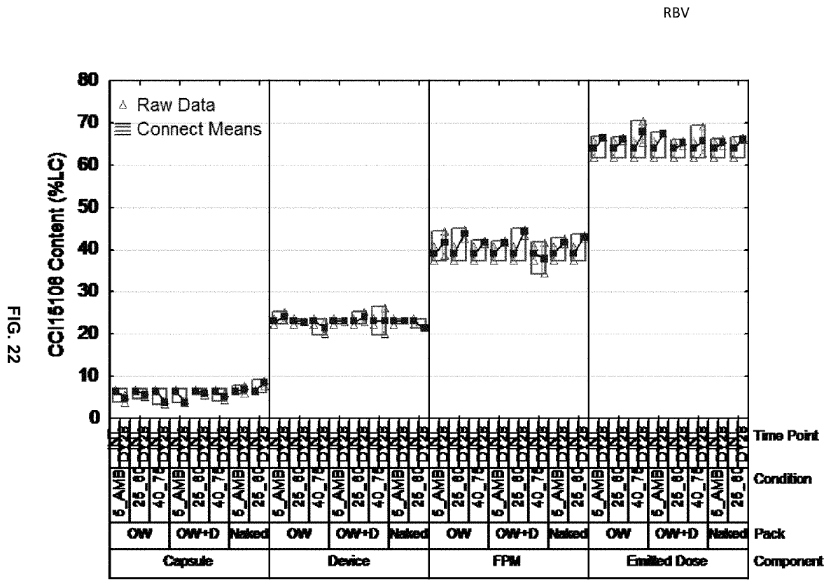

FIG. 23 depicts a Variability Plot showing Fine Particle Mass (FPM) Stability data up to DY28 for 30mg RBV Capsules using the Monodose RS01 Device at 60 L/min for 4 seconds (utilizing the crystalline 99:1 RBV: PVOH, 0.9×1 μm cylinder PRINT formulation). FIG. 23 shows FPM data generated at all conditions (5° C./Amb RH, 25° C./60% RH, 40° C./75% RH, 50° C./Amb RH, C-5° C./40). Note that only 5° C./Amb RH, 25° C./60% RH is available for Naked storage. Fine Particle Mass (FPM)=Sum of Stages 3, 4 & 5 at 60 L/min, equivalent to 0.94-4.46μm aerodynamic particle size distribution range expressed as % of 30 mg RBV per capsule. Mean results for all pack type/ storage conditions are within 39-49% Label Claim (% LC) up to 28 days. The plot shows that across the different pack types and/or conditions up to DY28, there are no significant differences shown for Fine Particle Mass (FPM).

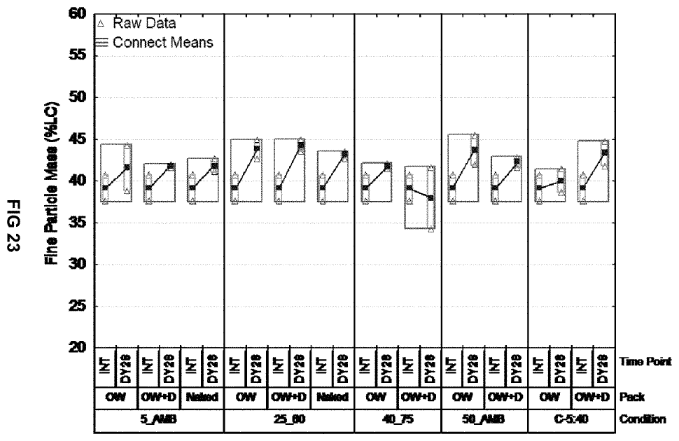

FIG. 24 shows a Variability Plot showing Fine Particle Fraction (FPF) Stability data up to DY28 for 30 mg RBV Capsules using the Monodose RS01 Device at 60 L/min for 4 seconds (utilizing the crystalline 99:1 RBV:PVOH, 0.9×1um cylinder PRINT formulation). FIG. 24 shows FPF data generated at all conditions (5° C./Amb RH, 25° C./60% RH, 40° C./75% RH, 50° C./Amb RH, C-5° C./40). Note that only 5° C./Amb RH, 25° C./60% RH is available for Naked storage. Fine Particle Fraction (FPF)=Particles<5 μm expressed as % of 30 mg RBV per capsule. Mean results for all pack type/storage conditions are within 43-54% Label Claim (% LC) up to 28 days. FIG. 24 demonstrates that across the different pack types and/or conditions up to DY28, there are no significant differences shown for Fine Particle Fraction (FPF).

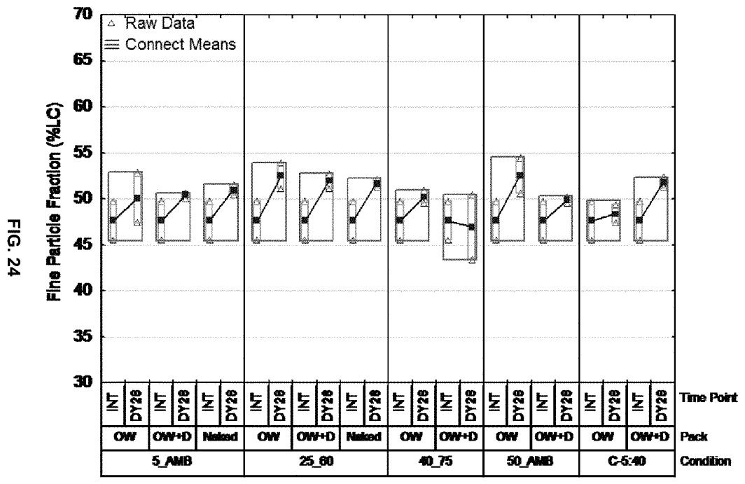

FIG. 25 depicts a Variability Plot showing Emitted Dose Stability data up to DY28 for 30mg RBV Capsules using the Monodose RS01 Device at 60 L/min for 4 seconds (utilizing the crystalline 99:1 RBV:PVOH, 0.9×1 μm cylinder Liquidia PRINT formulation). FIG. 25 shows Emitted Dose data generated at all conditions (5° C./Amb RH, 25° C./60% RH, 40° C./75% RH, 50° C./Amb RH, C-5° C./40). Note that only 5° C./Amb RH, 25° C./60% RH is available for Naked storage. Emitted Dose has been taken from NGI Totals (Sum of stages Throat, Stages 1-7, MOC, External Filter) expressed as % of 30mg RBV per capsule. Mean results for all pack type/storage conditions are within 64-68% Label Claim (% LC). This data demonstrates that across the different pack types and/or conditions up to DY28, there are no significant differences shown for Emitted Dose.

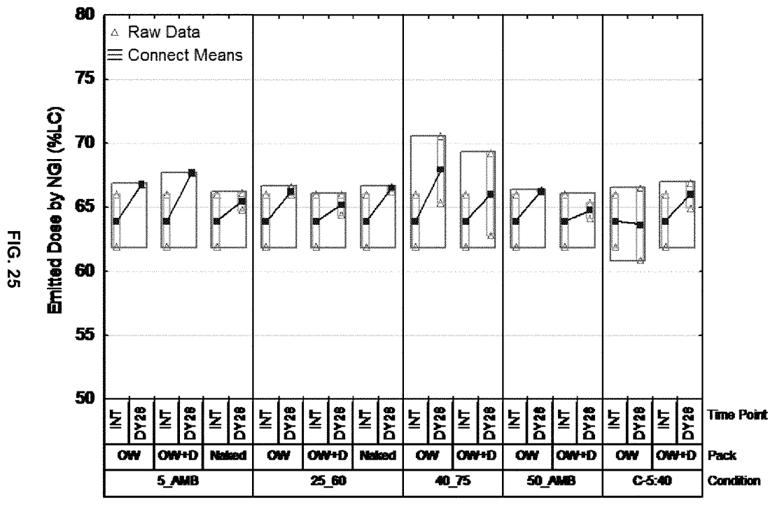

FIG. 26: depicts Stage Deposition Plot showing deposition difference for a crystalline RBV PRINT formulation (99:1 RBV:PVOH, 0.9×1 μm cylinder) after storage of capsules naked at 5° C./Amb and 25° C./60% RH when tested by the same Monodose RS01 device type and flow rate (60 L/min for 4 seconds). FIG. 26 shows stage deposition data generated at Initial (INT) and DY28 after capsule storage naked at 5° C./Amb RH and 25° C./60% RH. FIG. 26 shows that with the naked pack type, which is unprotected from the exposed environment of the testing chamber, there are no significant differences in deposition, thus highlighting the lack of agglomeration or other effects which may result in changes in aerosolisation efficiency are observed.

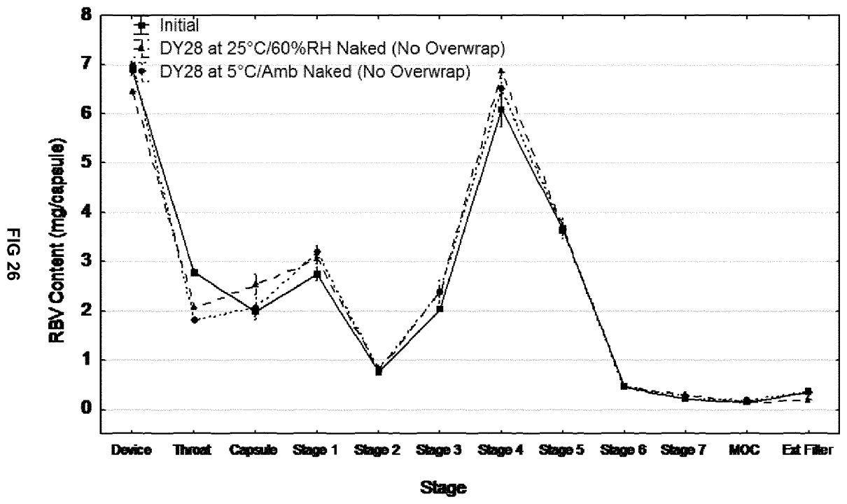

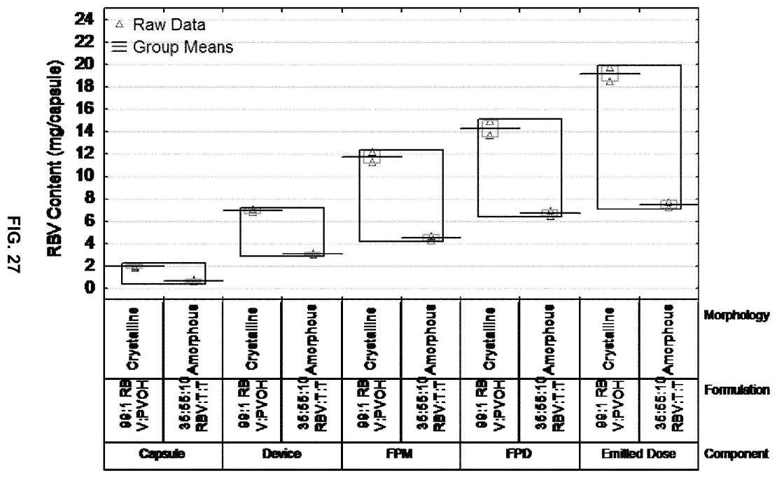

Data has been provided in FIG. 27 and FIG. 28 show the effects of formulation (amorphous RBV vs crystalline) on the aerosol performance and lung delivery of the RBV product (as 30.3 mg capsules of crystalline 99:1 RBV:PVOH, 0.9×1 μm cylinder RBV PRINT particles; and amorphous (35:55:10 RBV:Trehalose:Trileucine, 0.9×1μm cylinder) PRINT particles. All data is expressed as milligrams per capsule (mg/capsule) to show absolute distributions in each stage or summary group.

FIG. 27 is a Variability Plot showing Capsule & Device Deposition, Fine Particle Mass and Emitted Dose by NGI Stability data up to DY28 for 30 mg RBV Capsules using the Monodose RS01 Device at 60 L/min for 4 seconds (utilizing the crystalline 99:1 RBV:PVOH, 0.9×1 μm cylinder Liquidia PRINT formulation). FIG. 27 shows Capsule deposition, Device deposition, Fine Particle Mass, Fine Particle Dose and Emitted Dose data generated using capsules filled at the same nominal fill weight (30.3 mg) through the Monodose RS01 device for both Crystalline (99:1 RBV:PVOH) and Amorphous (35:55:10 RBV:Trehalsoe:Trileucine) PRINT formulations of the same 0.9×1 μm cylinder morphology. Data has been expressed as milligrams per capsule (mg/capsule) to show the absolute deposition comparison between the two formulations. Fine Particle Dose (FPD)=mg (of particles)<5 μm.

The data expressed in FIG. 27 indicate that for a single capsule filled with 30.3 mg of each formulation, the dose delivered to the lung is significantly higher for the crystalline 99:1 RBV:PVOH formulation when compared with the amorphous 35:55:10 RBV:Trehalose:Trileucine due to approximately 3x drug load increase possible in the crystalline formulation.

As will be appreciated, the implication of this loading advantage is that the volume of material required to deliver a dose of RBV with the crystalline formulation is approximately 1/3 of that required with the amorphous form. A single capsule of crystalline RBV in a 99% RBV formulation is the equivalent of approximately 3 capsules of the amorphous formulation.

FIG. 28. is a Stage Deposition Plot showing difference between crystalline (99:1 RBV:PVOH, 0.9×1 μm cylinder) and amorphous (35:55:10 RBV:Trehalose:Trileucine, 0.9×1 μm cylinder) Liquidia PRINT formulations when tested by the same Monodose RS01 device type and flow rate (60 L/min for 4 seconds). FIG. 28 shows stage deposition data generated using capsules filled at the same nominal fill weight (30.3mg) through the Monodose RS01 device for both Crystalline (99:1 RBV:PVOH) and Amorphous (35:55:10 RBV:Trehalsoe:Trileucine) Liquida PRINT formulations of the same 0.9×1 μm cylinder morphology. Data has been expressed as milligrams per capsule (mg/capsule) to show the absolute deposition in each stage.

FIG. 28 shows that RBV deposition is either the same or greater for crystalline PRINT particle formulations for all stages, but especially in the areas of the lung corresponding to Stages 3, 4 and 5 of the NGI. This allows easier patient administration as fewer capsules are required (with fewer inhalations). It also has the advantage of dosing the active pharmaceutical ingredient with minimal excipient dosing.

### Example 12

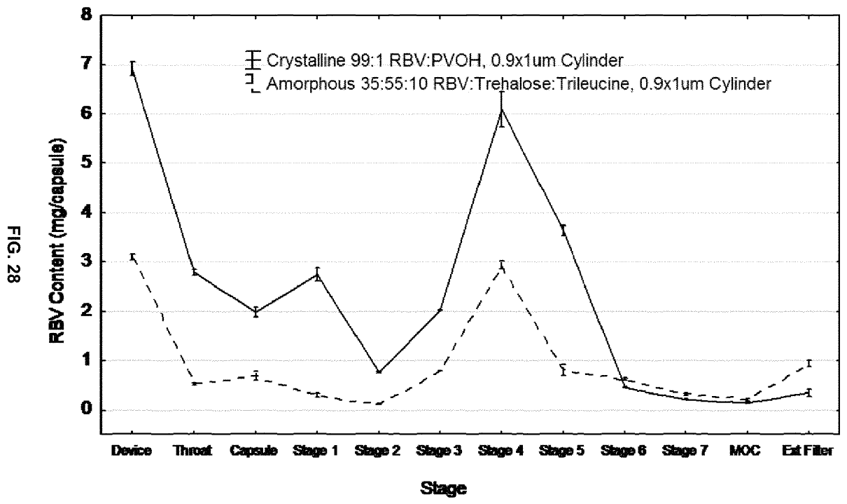

FIG. 29 shows NGI deposition data for 99:1 RBV:PVOH after 2 months of storage at various conditions (bars from left to right in the graph for each NGI stage represent samples stored for 2 months at (1) −20 degrees C.; (2) 25° C./60% RH in a closed container; (3) 25° C./60% RH in an open container, and; (4) 40° C. N=2, except for −20° C. condition. Various characterization data is shown in Tables 25-27 for the formulation, showing high RBV loading, peak purity, particle compositions were within respirable size (less than 5 μm), high emitted dose and Fine Particle Fractions for the 99:1 composition.

TABLE 25

| | RBV CONTENT | | RBV LC | MMAD | | | | ED | | FPF | |
|---|---|---|---|---|---|---|---|---|---|---|---|
| SAMPLE | (%) | | PEAK PURITY | (μM) | | GSG | | (% REC)** | | (% ED) | |
| −20° C. cl. | 102.4% | (1.1%) | 99.9% (0.0%) | 2.18 | (_)** | 2.67 | (—)** | 75% | (—)** | 74% | (—)** |
| 25/65 cl. | 100.8% | (2.4%) | 99.9% (0.0%) | 2.45 | (0.07) | 2.86 | (0.21%) | 74% | (1%) | 70% | (2%) |
| 25/65 o. | 84.2% | (25.6%)*** | 99.9% (0.0%) | 2.56 | (0.21) | 3.06 | (0.21) | 86% | (12%) | 68% | (1%) |
| 40° C. cl. | 97.0% | (0.6%) | 97.0% (0.6%) | 2.32 | (0.21) | 2.88 | (0.01) | 86% | (3%) | 69% | (1%) |

*NGI data obtained using a monodose device at 95 L/min (n = 2 unless otherwise stated)
**Second NGI sample for −20° C. did not actuate effectively
***One sample showed low recovery

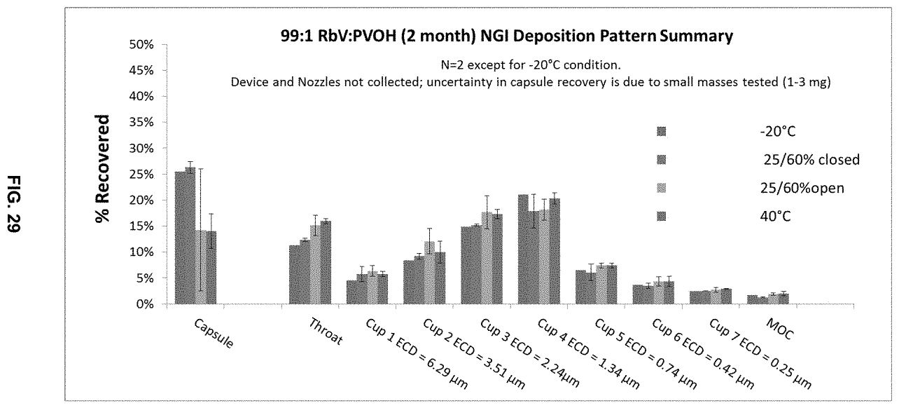

FIG. 30. depicts DSC data for 99:1 Ribavirin:PVOH (crystalline) on 2 month stability. This data shows no change in crystalline form after 2 months regardless of tested storage conditions (−20° C.; 25° C./60% RH in a closed container; 25° C./60% RH in an open container; and 40° C.). Tables 26 and 27 represent 99:1 Ribavirin:PVOH 3 and 6 month stability data.

TABLE 26

| | RBV content | RBV LC | MMAD | | ED | FPF |
|---|---|---|---|---|---|---|
| Sample | (%) | Peak Purity | (μm) | GSG | (% rec)** | (% ED) |
| −20° C. cl. | 96.2%^{G} | 99.9% (0.0%) | 2.8 (0.1) | 1.9 (0.0) | 73.5% (2.5%) | 65.5% (1.5%) |
| 25/65 cl. | 96.5%^{G} | 99.8% (0.0%) | 2.8 (0.1) | 1.9 (0.0) | 76.2% (1.2%) | 69.3% (3.1%) |
| 25/65 o. | 96.9%^{G} | 99.9% (0.0%) | 2.8 (0.1) | 1.9 (0.0) | 73.1% (1.2%) | 66.1% (0.2%) |
| 40° C. cl. | 96.4%^{G} | 97.0% (0.6%) | 2.7 (0.1) | 1.9 (0.0) | 77.7% (7.9%) | 69.6% (2.8%) |

TABLE 27

| | RBV content | | RBV LC | MMAD | | ED | FPF |
|---|---|---|---|---|---|---|---|
| Sample | (%) | | Peak Purity | (μm) | GSG | (% rec)** | (% ED) |
| −20° C. cl. | 102.4% | (1.1%) | 95.2%^{G} | 2.8 (0.1) | 2.0 (0.1) | 75.9% (0.7%) | 62.3% (0.7%) |
| 25/65 cl. | 100.8% | (2.4%) | 96.5%^{G} | 2.8 (0.0) | 2.0 (0.1) | 75.9% (5.4%) | 61.4% (1.0%) |
| 25/65 o. | 84.2% | (25.6%)*** | 97.2%^{G} | 3.2 (0.1) | 2.0 (0.1) | 74.0% (2.1%) | 58.2% (2.8%) |
| 40° C. cl. | 97.0% | (0.6%) | 96.6%^{G} | 2.8 (0.1) | 2.0 (0.0) | 73.9% (1.3%) | 65.5% (1.6%) |

### Example 13

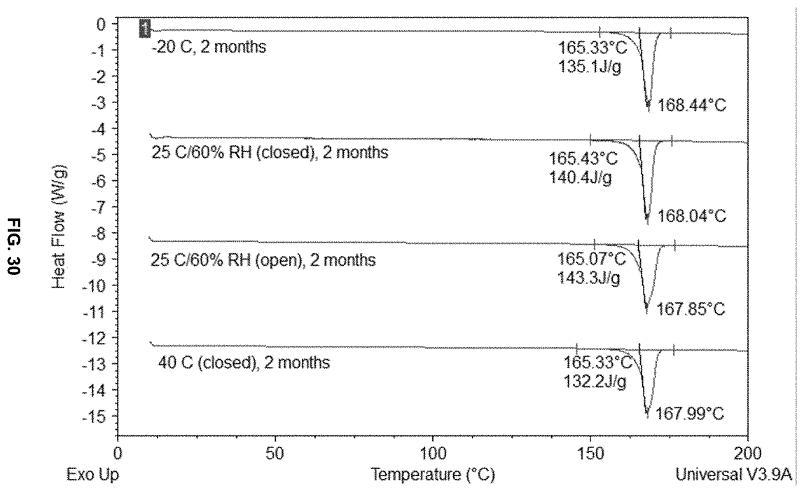

FIG. 31 shows NGI deposition data for 97:3 RBV:PVOH after 6 months of storage under various conditions (bars from left to right in the graph for each NGI stage represent samples stored for 2 months at (1) −20° C. in a closed container; (2) 25° C./60% RH in a closed container; (3) 25° C./60% RH in an open container, and; (4) 40° C. closed. N=2, except for −20° C. condition. Performance measures are tabulated below in Table 28.

TABLE 28

| | RBV content | RBV LC | MMAD | | ED | FPF |
|---|---|---|---|---|---|---|
| Sample | (%) | Peak Purity | (μm) | GSG | (% rec)** | (% ED) |
| −20° C. cl. | 96.2% (2.0%) | 99.5% (0.6%) | 1.55 (0.03) | 1.90 (0.00) | NA | 88% (0%) |
| 25/65 cl. | 93.5% (4.5%) | 99.9% (0.0%) | 1.60 (0.14) | 1.99 (0.02) | NA | 87% (2%) |
| 25/65 o. | 99.9% (1.4%) | 99.9% (0.0%) | 2.20 (0.14) | 1.90 (0.01) | NA | 75% (1%) |
| 40° C. cl. | 95.8% (1.5%) | 99.9% (0.0%) | 1.72 (0.04) | 1.97 (0.01) | NA | 85% (1%) |

*NGI data obtained using a monodose device at 95 L/min (n = 2 unless otherwise stated)
**Emitted doses not obtained since capsules and devices were not assayed

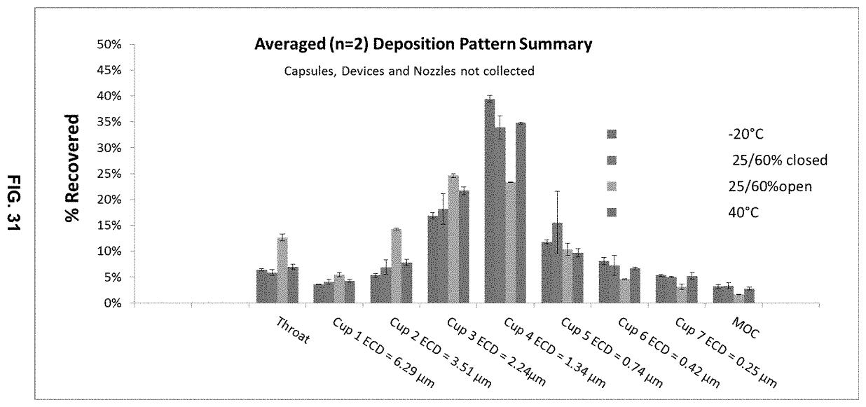

FIG. 32 depicts DSC data for 97:3 Ribavirin:PVOH (crystalline) material after 6 month stability under which samples stored at (1) −20° C. in a closed container; (2) 25° C./60% RH in a closed container; (3) 25° C./60% RH in an open container, or (4) 40° C. closed. Crystalline 97:3 Ribavirin:PVOH shows no change in crystalline form after 6 months. (ΔH_{Form I}/ΔH_{Form II}*100% for −20° C. closed samples=0.44%; for 25/65 closed samples=0.21%; for 25/65 open samples=0.17%, and for 40° C. closed samples=0.15%).

### Example 14

To investigate the effect of RBV when administered as a spray dried formulation, a study was conducted on 6 groups of 3 male rats/group that received a single 15 minute exposure via nose only inhalation of a spray dried particle containing 35% w/w Ribavirin, 55% trehalose and 10% (w/w) leucine. The nominal inhaled dose was 2mg/kg, and the actual inhaled dose: 2.036mg/kg/day. The particle size is indicated in Table 29.

In the study, a group of animals was killed immediately after the end of the exposure period and subsequently at 0.5, 1, 2, 6 and 24 hours after the end of the exposure period. From these animals, plasma and lung samples were collected for determination of RBV concentration. Spray dried particles containing 35% w/w Ribavirin with 55% w/w trehalose and 10% (w/w) leucine were prepared.

A target inhaled dose of 2 mg/kg ribavirin (single dose) was selected for each group as this represented an approximate rat equivalent dose of a possible human total dose of 20 mg (assuming a human body weight of 60 Kg and scaling factors of 6 [rat] and 37 [humans] when converting from mg/kg to mg/m²). The inhalation exposure system consisted of snout only, flow through ADG inhalation exposure chambers. The animals were restrained in polycarbonate restraint tubes which were attached to the chambers. Aerosols were generated into the top section of the inhalation chamber using a capsule based aerosol generator (CBAG). The diluent air connection located at the top of the exposure chamber remained open throughout the exposure period to balance airflow and maintain the chamber at near ambient pressure. The atmosphere in the chamber was extracted to ensure a flow of aerosol through the chamber.

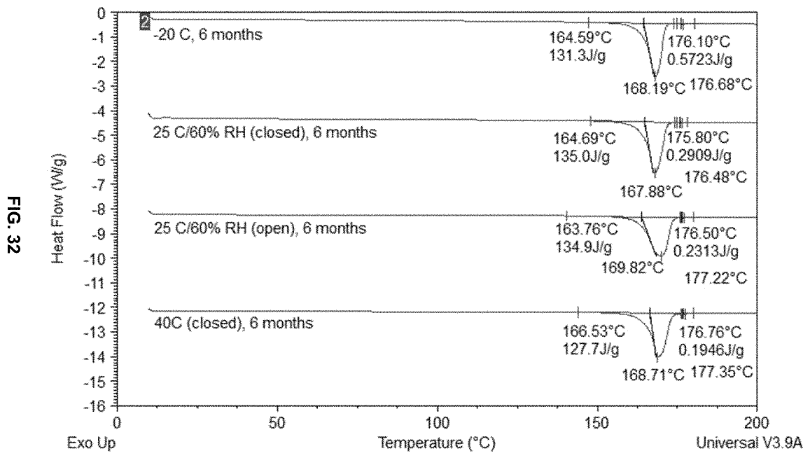

The results, shown in Table 30, indicate that it is possible to generate aerosols of ribavirin using spray dried particles containing 35% w/w Ribavirin with 55% w/w trehalose. Ribavirin was quantified in plasma and lung samples. FIG. 33 shows the Composite Mean Plasma and Lung Concentrations of Ribavirin Following a Single Inhalation Administration of a Spray Dried Formulation of Ribavirin at a Target Inhaled Dose of 2 mg/kg to Male Rats.

Table 29 details the Particle Size Distribution Results for Spray dried RBV particles RBV:Trehalose:Leucine (35:55:10% w/w) Particle Compositions

TABLE 29

| | Mean achieved | | |
|---|---|---|---|
| | chamber aerosol | MMAD | |
| Group No. | concentration (μg/L) | (μm) | GSD |
| PSD 1 | 194 (analytical | 1.3 | 3.0 |
| PSD 2 | 194 (analytical) | 1.2 | 3.5 |

TABLE 30

| | | Lung¹ | Lung¹ | Plasma¹ | Plasma¹ |
|---|---|---|---|---|---|
| | MMAD | Cₘₐₓ | AUC(0-t) | Cₘₐₓ | AUC(0-t) |
| | (±GSD) | (μg/g)/ | (μg*h/g)/ | (μg/mL)/ | (μg*h/mL)/ |
| | μm | (mg/kg) | (mg/kg) | (mg/kg) | (mg/kg) |
| Spray dried dispersion | 3.0 (1.3) | 0.688 | 0.363 | 0.0468 | 0.0737 |
| (35:55:10 w/w | 3.5 (1.2) | | | | |
| Ribvairin:Trehalose:Leucine) | | | | | |

¹Dose-normalized Cmax and AUC₀₋ₜ. Dose delivered: 2.036 mg/kg (measured by analytical method).

Studies in inhalation Pharmacokinetic (PK) rat lung models, represented by e.g., Tables 14 and 30, show that RBV PRINT particles achieve a greater than 8× increase in dose-normalized lung Cmax, and a greater than 11× increase in dose-normalized lung AUC₀₋ₜ when compared with data generated with Spray dried RBV particles (SDD).

As will be appreciated by the above, the present invention provides advantages in ribavirin therapy. As a dry powder, these RBV containing compositions, whether presented as an amorphous or crystalline solid, potentially avoid the problems of bronchospasm associated with nebulized aqueous formulations, especially in COPD patients. Current clinical data in human healthy volunteers suggest the dry-powder formulation containing 35% ribavirin in 55% trehalose and 10% trileucine does not cause abnormalities in pulmonary function in these subjects. Bronchospasm is explained by the Walsh, Respi Care (2016) article mentioned previously, which suggests the mechanism of bronchospasm is irritation caused by the hypotonic solution, and in which the authors suggest replacing the sterile water for dilution of the active with saline. Because the compositions of fabricated particles of the present invention are not a hypotonic solution, this may provide patients with a clinical benefit of avoiding this unfavorable reaction.

Further benefit of the particle compositions of the present invention include their ability to deliver a required dosage in a more concentrated manner. The required dosage can be delivered in a shorter time span when compared to nebulized formulations (Virasol®). As a powder inhaled from an inhaler is directly introduced to a patient's lungs in one to a few inhalation cycles, treatment time is reduced to seconds rather than many minutes to hours, largely avoiding the environmental exposure caregivers experience with dilute, nebulized formulations, an advantage with teratogenic compounds such as ribavirin.

While both crystalline and amorphous forms of templated particles of RBV described herein offer advantages over the available nebulized forms, the crystalline templated RBV particles of the present invention, which may be loaded with greater than 90% crystalline RBV, offers loading advantages over the amorphous RBV PRINT formulations described herein. This reduction in the volume of inhaled powder to deliver a desired dose of ribavirin promotes convenience and compliance in effective treatment, while potentially reducing pulmonary irritation. These and many other advantages will be appreciated by those of skill in the art based upon the description provided herein.

What is claimed is:

1. A composition comprising fabricated particles comprising ribavirin and one or more optional excipients.

2. The pharmaceutical composition according to claim 1, wherein the fabricated particles have a mass median aerodynamic diameter (MMAD) ranging from about 0.5 μm to about 6 μm.

3. The pharmaceutical composition according to claim 1, wherein the fabricated particles have a mass median aerodynamic diameter (MMAD) ranging from about 0.5 μm to about 3 μm.

4. The pharmaceutical composition according to claim 1, wherein the fabricated particles have a mass median aerodynamic diameter (MMAD) ranging from about 1.0 μm to about 2.5 μm.

5. The pharmaceutical composition according to claim 1, wherein the fabricated particles have a mass median aerodynamic diameter (MMAD) ranging from about 1.5 μm to about 2.5 μm.

6. The pharmaceutical composition according to claim 1, wherein the fabricated particles have a mass median aerodynamic diameter (MMAD) ranging from about 1.5 μm to about 2.0 μm.

7. The pharmaceutical composition according to any one of claims 1-6, wherein the fabricated particles have a substantially uniform shape.

8. The pharmaceutical composition according to any one of claims 1-7, wherein each fabricated particle is substantially non-spherical.

9. The pharmaceutical composition according to any one of claims 1-8, wherein each fabricated particle is substantially non-porous.

10. The pharmaceutical composition according to any one of claims 1-9, wherein each fabricated particle comprises an amount of ribavirin ranging from about 0.04 picograms to about 4.5 picograms total ribavirin weight per particle.

11. The pharmaceutical composition according to any one of claims 1-10, wherein each fabricated particle comprises a percentage loading of ribavirin ranging from about 1% w/w to about 100% w/w.

12. The pharmaceutical composition according to claim 11, wherein each fabricated particle comprises a percentage loading of ribavirin ranging from about 10% w/w to about 55% w/w.

13. The pharmaceutical composition according to claim 12, wherein each fabricated particle comprises a percentage loading of ribavirin ranging from about 20% w/w to about 50% w/w. 14. The pharmaceutical composition according to claim 11, wherein each fabricated particle comprises a percentage loading of ribavirin in a range of from about 95% w/w to about 99% w/w.

15. The pharmaceutical composition according to any one of claims 1-10, wherein each fabricated particle comprises a percentage loading of ribavirin of greater than about 10% w/w.

16. The pharmaceutical composition according to claim 15, wherein each fabricated particle comprises a percentage loading of ribavirin of greater than about 20% w/w.

17. The pharmaceutical composition according to claim 15, wherein each fabricated particle comprises a percentage loading of ribavirin of greater than about 30% w/w.

18. The pharmaceutical composition according to claim 15, wherein each fabricated particle comprises a percentage loading of ribavirin of greater than about 40% w/w.

19. The pharmaceutical composition according to claim 15, wherein each fabricated particle comprises a percentage loading of ribavirin of greater than about 50% w/w.

20. The pharmaceutical composition according to claim 15, wherein each fabricated particle comprises a percentage loading of ribavirin of greater than about 60% w/w.

21. The pharmaceutical composition according to any one of claims 1-10, wherein each fabricated particle comprises a percentage loading of ribavirin of about 99% w/w.

22. The pharmaceutical composition according to any one of claims 1-10, wherein each fabricated particle comprises a percentage loading of ribavirin of about 35% w/w.

23. The pharmaceutical composition according to any one of claims 1-22, wherein the excipient comprises a carbohydrate, an amino acid, a polypeptide, a synthetic polymer, or mixtures thereof.

24. The pharmaceutical composition according to claim 23, wherein the excipient comprises a mixture of a carbohydrate and an amino acid or a polypeptide.

25. The pharmaceutical composition according to claim 23, wherein the excipient is selected from one or more of trehalose, lactose, leucine, di-leucine, tri-leucine, dextran, cyclodextran, maltose, sucrose, glucose, sorbitol, erythritol, mannitol, dextrose, maltitol, maltose, mannilol, raffinose, galactose, xylose, ribose, xylitol, tryptophan, tyrosine, phenylalanine, and maltodextrin.

26. The pharmaceutical composition according to claim 24, wherein the excipient comprises a mixture of trehalose and trileucine.

27. The pharmaceutical composition according to claim 25, wherein the lactose is a lactose monohydrate.

28. The pharmaceutical composition according to claim 25 or claim 26, wherein the trehalose is trehalose dihydrate.

29. The pharmaceutical composition according to claim 25, 26, or 28, wherein each fabricated particle comprises a percentage loading of trehalose in a range of from about 50% w/w to about 94% w/w.

30. The pharmaceutical composition according to claim 29, wherein each fabricated particle comprises a percentage loading of trehalose in a range of from about 50% w/w to about 60% w/w.

31. The pharmaceutical composition according to any one of claim 25, 26, or 28, wherein each fabricated particle comprises a percentage loading of trehalose that is greater than about 50% w/w.

32. The pharmaceutical composition according to claim 30, wherein each fabricated particle comprises a percentage loading of trehalose that is about 55% w/w.

33. The pharmaceutical composition according to claim 25 or claim 26, wherein each fabricated particle comprises a percentage loading of trileucine in a range of from about 5% w/w to about 15% w/w.

34. The pharmaceutical composition according to claim 33, wherein each fabricated particle comprises a percentage loading of trileucine in a range of from about 7% w/w to about 12% w/w.

35. The pharmaceutical composition according to claim 25 or claim 26, wherein each fabricated particle comprises a percentage loading of trileucine that is greater than about 5% w/w.

36. The pharmaceutical composition according to claim 25 or claim 26, wherein each fabricated particle comprises a percentage loading of trileucine that is about 10% w/w.

37. The pharmaceutical composition according to any one of claims 1-36, wherein the composition is a dry powder composition.

38. The pharmaceutical composition according to any one of claims 1-37, wherein the fabricated particles are formed by molding the ribavirin and excipient in mold cavities.

39. The pharmaceutical composition according to claim 38, wherein the fabricated particles are formed by molding the ribavirin and excipient in polymeric molds. 40. The pharmaceutical composition according to claim 37, wherein the dry powder is encapsulated in a capsule or blister.

41. The pharmaceutical composition according to claim 40, wherein the capsule comprises hydroxypropyl methylcellulose, gelatin, hypromellose, pullulan and starch materials.

42. The pharmaceutical composition according to any one of claim 37, 40, or 41, wherein the dry powder has a moisture content below about 10% by weight water.

43. The pharmaceutical composition according to claim 42, wherein the dry powder has a moisture content below about 1% by weight water.

44. The pharmaceutical composition according to any one of claims 1-43, wherein the ribavirin is substantially amorphous.

45. The pharmaceutical composition according to any one of claim 37, 40, 41, 42, or 43, wherein: the dry powder has a bulk density of less than about 2.5 g/cm³.

46. The pharmaceutical composition according to claim 45, wherein: the dry powder has a bulk density of less than about 1.0 g/cm³.

47. The pharmaceutical composition according to any one of claims 1-46, wherein the fabricated particles comprise two substantially parallel surfaces.

48. The pharmaceutical composition according to claim 47, wherein each substantially parallel surface has a substantially equal linear dimension.

49. The pharmaceutical composition according to any one of claims 1-48, wherein the fabricated particles in cross-section comprise two substantially parallel surfaces and one or more substantially non-parallel surfaces.

50. The pharmaceutical composition according to claim 26, wherein the wherein the fabricated particles comprise a ratio of ribavirin to trileucine of greater than about 5 to about 1.

51. The pharmaceutical composition according to claim 26, wherein the fabricated particles comprise a ratio of ribavirin to trehalose to trileucine of about 3.5 to about 5.5 to about 1.

52. The pharmaceutical composition according to claim 26, wherein the fabricated particles comprise a ratio of ribavirin to trileucine of greater than about 3 to about 0.5, and wherein the fabricated particles comprise a ratio of trehalose to trileucine of greater than about 4 to about 0.5.

53. The pharmaceutical composition according to claim 26, wherein the fabricated particles comprise a ratio of trehalose to trileucine of greater than about 4 to about 0.5, and wherein the ratio of trehalose to trileucine substantially prevents pH dependent degradation of the ribavirin.

54. An inhaler comprising a pharmaceutical composition comprising dry powder fabricated particles comprising ribavirin and an excipient, wherein the fabricated particles have a substantially uniform distribution of solid material throughout, and are substantially non-porous, and have a mass median aerodynamic diameter (MMAD) ranging from about 0.5 μm to about 6 μm.

55. The inhaler according to claim 54, wherein the fabricated particles have a substantially uniform shape.

56. The inhaler according to claim 54 or claim 55, wherein each fabricated particle is substantially non-spherical.

57. The inhaler according to any one of claims 54-56, wherein each fabricated particle is substantially non-porous.

58. The inhaler according to any one of claims 54-57, wherein one emitted dose of the composition into the lungs of a human subject provides (i) 30 mg of ribavirin to the lungs of the subject, (ii) a Cₘₐₓ value of ribavirin that is less than about 0.5 μg/m L, in the human subject's systemic bloodstream, and (iii) a Tₘₐₓ greater than 3 hours in the human subject's systemic bloodstream.

59. The inhaler according to any one of claims 54-57, wherein one emitted dose of the composition into the lungs of a human subject provides a Cₘₐₓ value of ribavirin that is less than about 0.30 μg/mL in the human subject's systemic bloodstream.

60. The inhaler according to any one of claims 54-57, wherein one emitted dose of the composition into the lungs of a human subject provides a Cₘₐₓ value of ribavirin that is less than about 0.25 μg/mL in the human subject's systemic bloodstream.

61. The inhaler according to any one of claims 54-57, wherein one emitted dose of the composition into the lungs of a human subject provides a Cₘₐₓ value of ribavirin that is less than about 0.10 μg/mL in the human subject's systemic bloodstream.

62. The inhaler according to any one of claims 54-57, wherein one emitted dose of the composition into the lungs of a human subject provides a Cₘₐₓ value of ribavirin that is less than about 0.05 μg/mL in the human subject's systemic bloodstream.

63. The inhaler according to any one of claims 54-57, wherein one emitted dose of the composition into the lungs of a human subject provides an AUC value of ribavirin that is less than about 20 μg*hr/mL in the human subject's systemic bloodstream.

64. The inhaler according to claim 63, wherein one emitted dose of the composition into the lungs of a human subject provides an AUC value of ribavirin that is less than about 10 ug*hr/mL in the human subject's systemic bloodstream.

65. The inhaler according to claim 64, wherein one emitted dose of the composition into the lungs of a human subject provides an AUC value of ribavirin that is less than about 5 μg*hr/mL in the human subject's systemic bloodstream.

66. The inhaler according to claim 65, wherein one emitted dose of the composition into the lungs of a human subject provides an AUC value of ribavirin that is less than about 3 μg*hr/mL in the human subject's systemic bloodstream.

67. The inhaler according to claim 66, wherein one emitted dose of the composition into the lungs of a human subject provides an AUC value of ribavirin that is less than about 2 μg*hr/mL in the human subject's systemic bloodstream.

68. The inhaler according to any one of claims 54-67, wherein the inhaler provides an emitted dose of ribavirin that is more than about 4 mg.

69. The inhaler according to claim 68, wherein the inhaler provides an emitted dose of ribavirin that is more than about 5 mg.

70. The inhaler according to claim 69, wherein the inhaler provides an emitted dose of ribavirin that is more than about 7 mg.

71. The inhaler according to claim 70, wherein the inhaler provides an emitted dose of ribavirin that is more than about 10 mg.

72. The inhaler according to claim 71, wherein the inhaler provides an emitted dose of ribavirin that is more than about 15 mg.

73. The inhaler according to claim 72, wherein the inhaler provides an emitted dose of ribavirin that is more than about 20 mg.

74. The inhaler according to claim 73, wherein the inhaler provides an emitted dose of ribavirin that is more than about 50 mg.

75. The inhaler according to any one of claims 54-57, wherein the amount of ribavirin present in one emitted dose of the dry powder composition is less than about 30 mg and wherein the dry powder composition provides: a maximum blood plasma concentration of ribavirin (Cmax) of less than about 0.50 μg/mL; an AUC₀₋₂₄ hour value of ribavirin of less than 25 μg*hr/mL; and wherein the Cₘₐₓ of ribavirin and the AUC₀₋₂₄ hour value are measured after a single pulmonary administration of the dry powder composition to a human subject.

76. A unit dosage form comprising a container containing a pharmaceutical composition according to any one of claims 1 to 53.

77. The unit dosage form of claim 76, wherein the container comprises a capsule.

78. The unit dosage form of claim 76, wherein the container comprises a blister pack.

79. A method of making the fabricated particles according to claims 1 of claim 2, comprising:
preparing a particle precursor solution of ribavirin and one or more optional excipients in a solvent;
casting the particle precursor solution into a film on a backing sheet;
drying the film by removing the solvent, retaining the particle precursor on the backing sheet;
nipping the dried film sheet containing the dried particle precursor with a mold in a heated nip roller, wherein the mold defines mold cavities;
flowing and solidifying the dried film particle precursor into the mold cavities where it remains on the backing sheet creating isolated uniform shaped particles of the particle precursor in the mold cavities;
removing the sheet from the mold wherein the isolated uniform shaped particles remain on the backing sheet and are removed from the mold cavities;
removing the isolated uniform shaped particles from the sheet; and
collecting the isolated uniform shaped particles.

80. The method according to claim 79, wherein the solvent is water.

81. The method according to claim 79 or claim 80, wherein the temperature of the nip ranges from about 20° C. to about 300° C.

82. The method according to any one of claims 79-81, wherein the pressure of the roller ranges from about 5 psi to about 80 psi.

83. The method according to any one of claims 79-82, wherein the speed of the roller is greater than 0 ft/min and ranges up to about 25 ft/min.

84. Fabricated particles comprising 1wt %-100wt % ribavirin made according to a process comprising:
preparing a particle precursor solution of ribavirin and one or more optional excipients in a solvent;
casting the particle precursor solution into a film on a backing sheet;
drying the film by removing the solvent, retaining the particle precursor on the backing sheet;
nipping the dried film sheet containing the dried particle precursor with a mold in a heated nip roller, wherein the mold defines mold cavities;
flowing and solidifying the dried film particle precursor into the mold cavities where it remains on the backing sheet creating isolated uniform shaped particles of the particle precursor in the mold cavities;
removing the sheet from the mold wherein the isolated uniform shaped particles remain on the backing sheet and are removed from the mold cavities;
removing the isolated uniform shaped particles from the sheet; and
collecting the isolated uniform shaped particles.

85. A method of ameliorating respiratory infection-induced exacerbations of a respiratory disorder in a subject comprising administering to the subject a therapeutically effective amount of the pharmaceutical composition according to claims 1-53.

86. A method of preventing respiratory infection-induced exacerbations of a respiratory disorder in a human subject comprising administering to the human subject a therapeutically effective amount of the pharmaceutical composition according to claims 1-53.

87. A method of reducing the incidence of respiratory infection-induced exacerbations of a respiratory disorder in a human subject comprising administering to the human subject a therapeutically effective amount of the pharmaceutical composition according to claims 1-53.

88. A method of reducing the severity of respiratory infection-induced exacerbations of a respiratory disorder in a human subject comprising administering to the human subject a therapeutically effective amount of the pharmaceutical composition according to claims 1-53.

89. The method according to any one of claims 85-88, wherein the respiratory disorder is a viral respiratory infection or a bacterial respiratory infection, or both.

90. A method of treating a respiratory viral infection in a human subject comprising administering to the human subject a therapeutically effective amount of the pharmaceutical composition according to claims 1-53.

91. A method of preventing a respiratory viral infection in a human subject comprising administering to the human subject a therapeutically effective amount of the pharmaceutical composition according to claims 1-53.

92. The method according to any one of claims 85-88, wherein the respiratory disorder is a chronic respiratory disorder.

93. The method according to any one of claims 85-88, wherein the respiratory disorder is selected from the group consisting of chronic obstructive pulmonary disease (COPD), asthma, and cystic fibrosis.

94. The method according to any one of claims 89-91, wherein the viral respiratory infection is selected from the group consisting of human rhinovirus (HRV), Respiratory Syncytial virus (RSV), Middle East Respiratory Syndrome (MERS), Severe Acute Respiratory Syndrome (SARS), influenza, parainfluenza, human metapneumovirus, adenovirus, coronavirus, and picornavirus.

95. The method according to any one of claims 85-94, wherein pharmaceutical composition is administered to the human subject by a dry powder inhaler.

96. The method according to any one of claims 85-94, wherein pharmaceutical composition is administered to the human subject by the inhaler according to any one of claims 54-75.

97. The method according to claim 96, wherein the pharmaceutical composition is administered from the inhaler in less than 16 inhalations or less per day over a course of therapy of less than 15 days.

98. The method according to claim 96, wherein the pharmaceutical composition is administered as two inhalations per day from the dry powder inhaler over 14 days.

99. A plurality of substantially uniformly shaped and sized particles, comprising:
an engineered shape;
wherein each of the particles of the plurality comprise, in cross-section, non-parallel lateral surfaces and parallel top and bottom surfaces;
wherein the size of each particle is less than about 10 μm in a broadest dimension; and
each particle of the plurality of particles comprises
(i) ribavirin; and
(ii) optionally an excipient.

100. The particles according to claim 99, wherein the engineered shape is selected from the group consisting of an angle, an edge, an arc, a vertex, and a point.

101. The particles according to claim 99, wherein the engineered shape, in top-plan view, comprises a three spoked particle.

102. The particles according to claim 99, wherein the engineered shape is selected from the group consisting of a cylinder, a trapezoid, a cone, a rectangle, and an arrow.

103. The particles according to any one of claims 99-103, wherein the broadest dimension is less than about 3 μm.

104. The particles according to any one of claims 99-103, wherein said excipient is selected from a group consisting of trehalose, trileucine, dextran, and lactose.

105. The particles according to claim 104, wherein said excipient is lactose.

106. The particles according to claim 105, wherein the percentage of lactose in a particle is between 40 and 80 weight percent.

107. The particles according to claim 106, wherein the percentage of lactose in a particle is 65 weight percent.

108. The particles according to claim 104, wherein said excipient is trehalose.

109. The particles according to claim 108, wherein the percentage of trehalose in a particle is between 40 and 80 weight percent.

110. The particles according to claim 109, wherein the percentage of trehalose in a particle is 65 weight percent.

111. The particles according to claim 104, wherein said excipient is trileucine.

112. The particles according to claim 111, wherein the percentage of trileucine in a particle is between 5 and 20 weight percent.

113. The particles according to claim 112, wherein the percentage of trileucine in a particle is 15 weight percent.

114. The particles according to any one of claims 99-113, wherein the percentage of ribavirin in a particle is between 25 and 50 weight percent.

115. The particles according to claim 114, wherein the percentage of ribavirin in a particle is 95-100 weight percent.

116. The particles according to any one of claims 108-110, further comprising trileucine.

117. The particles according to claim 116, comprising 55 weight percent trehalose and 10 weight percent trileucine.

118. A dose container containing a single dose of the pharmaceutical composition of any of claims 1 to 53, wherein the fill weight of the composition of a single dose is less than about 30 mg.

119. A dose container, containing a single dose of the pharmaceutical composition of any of claims 1 to 53, wherein the density is about 0.18 g/cm³.

120. The dose container according to claim 118 or claim 119, wherein the dose container is a capsule or blister, and the pharmaceutical composition is contained therein.

121. The dose container according to claim 120, wherein the dose container is a blister, said blister comprising a base sheet defining a pocket, and a lid sheet sealingly attached thereto.

122. The dose container of claim 121, wherein the pocket is accessed by puncturing the base sheet or lid sheet, or both.

123. The dose container of claim 121, wherein the pocket is accessed by pealing the lid sheet from the base sheet.

124. A method of treatment comprising delivery of 30 mg of ribavirin from a blister or capsule filled with a specific amount of ribavirin, as measured by FPF or FPM in NGI, where the plasma Cₘₐₓ does not exceed 0.03 μg/mL at 4 hours after delivery.

125. A method of treatment comprising delivery of 30 mg of ribavirin from a blister or capsule filled with a specific amount of ribavirin, as measured by FPF or FPM in NGI, where ribavirin is delivered with a single unit inhaler provided with 4 capsules and where each capsule contains less than 30 mg of ribavirin.

126. A method of treating a human subject with an active pharmaceutical ingredient;
comprising:
dosing through a dry powder inhaler a plurality of particles, wherein each particle of the plurality comprises:
a substantially identical three-dimensional shape;
at least 20 wt % ribavirin ;
a largest linear dimension within 5% or less of each plurality member;
wherein the plurality has an MMAD of less than 6 μm; and
a single dose provides less than 60 mg of ribavirin to the lungs of the subject.

127. A composition comprising fabricated particles comprising ribavirin and one or more excipients.

128. A pharmaceutical composition comprising fabricated particles consisting of ribavirin and polyvinyl alcohol.

129. A pharmaceutical composition comprising fabricated particles consisting of ribavirin.

130. A crystalline ribavirin dry power inhaled composition, comprising: a plurality crystalline ribavirin particles;
wherein each particle comprises substantially equivalent volume and three-dimensional shape;
wherein each particle comprises between about 95% to 100% ribavirin; and
wherein the composition does not include a carrier/dilluent.

131. The composition of claim 130, wherein the composition provides greater than about 10 times lung Cmax compared to equivalent drug does of micronized ribavirin.

132. A composition comprising:
a plurality of fabricated particles wherein each particle of the plurality comprises:
a non-spherical engineered shape; and
95 to 99.5 wt % ribavirin and an excipient,
wherein the ribavirin is dispersed substantially throughout the particle and further
wherein at least 95 wt % of the ribavirin is present as polymorph Form II.

133. The composition of claim 132, wherein at least 99 wt % of the ribavirin is polymorph form II.

134. The composition of claim 132, wherein the excipient comprises polyvinyl alcohol.

135. The composition of claim 132, wherein the plurality of particles has a mass medial aerodynamic diameter (MMAD) ranging from about 0.5 μm to about 3 μm.

136. The composition of claim 132, wherein the plurality of particles has a mass median aerodynamic diameter (MMAD) ranging from about 1.0 μm to about 3 μm.

137. The composition of claim 132, wherein each particles of the plurality of particles has a substantial cylindrical shape having a width about 0.9 micrometers and a height about 1 micrometer.

138. The composition of claim 132, wherein each particles of the plurality of particles has a substantial cylindrical shape having a width selected between 1 and 3 micrometers and a height selected from between about 1 and 3 micrometers.

139. The composition of claim 132, further wherein the non-spherical engineered shape comprises two substantially parallel surfaces.

140. The composition of claim 139, wherein the two substantially parallel surfaces have substantially equal linear dimensions.

141. The composition of claim 132, further wherein the non-spherical engineered shape comprises, in cross-section, two substantially parallel lateral sides and substantially parallel top and bottom sides.

142. The composition of claim 141, wherein the two substantially parallel lateral sides comprise linear dimensions which are substantially equal.

143. The composition of claim 141, wherein the substantially parallel top and bottom sides comprise linear dimensions which are substantially equal.

144. The composition of claim 143, further comprising substantially parallel top and bottom sides having linear dimensions which are substantially equal.

145. A method of making a crystalline polymorph form controlled ribavirin dry powder inhalation particles, comprising:
partially dissolving crystalline ribavirin powder of a preferred polymorph form in an aqueous wetting agent to make a ribavirin solution;
casting the ribavirin solution onto a web and drying the solution;
mating the cast dried ribavirin solution with a polymer mold defining mold cavities, such that the cast dried ribavirin solution enters the cavities of the mold yet retains a flash layer interconnecting the cavities of the mold in communication; and
annealing the ribavirin solution in the cavities of the mold, wherein crystallization propagates between mold cavities through the flash layer to crystalize the dried ribavirin solution into ribavirin particles substantially mimicking the mold cavity shape and comprising greater than 95% polymorph form control.

146. The method of claim 145, wherein the ribavirin polymorph form is Form II.

147. The method of claim 145, wherein the ribavirin particles have a diameter 5 times smaller than median size of the ribavirin powder.

148. The method of claim 145, wherein the ribavirin particles comprise greater than 99% polymorph Form II ribavirin.

149. The method of claim 145, wherein the wetting agent is PVOH.

150. The method of claim 149, wherein the PVOH comprises less than about 5 wt % of the ribavirin particle.

151. The method of claim 149, wherein the PVOH comprises less than about 1.5 wt % of the ribavirin particle.

152. A pharmaceutical composition comprising dry powder particles which are from about 1 to about 10 microns in size and which comprise crystalline ribavirin, wherein said crystalline ribavirin is present in the amount of 0.001% to 99.9% by weight of said pharmaceutical composition.

153. The pharmaceutical composition of claim 153, further comprising PVOH.

154. The pharmaceutical composition of claim 152, wherein said ribavirin is present in the io amount of 90.0% to 99.5% by weight of said pharmaceutical composition.

155. A pharmaceutical composition of claim 152, wherein said ribavirin is the Form 2 polymorph, characterized by an XRPD profile comprising at one peak selected from the group consisting of about 12.0, 18.2, 23.0, and 25.4 degrees 2 Theta.

156. A pharmaceutical composition of claim 152, wherein said ribavirin is the Form 2 polymorph, characterized by an XRPD profile comprising peaks at about 12.0, 18.2, 23.0, and 25.4 degrees 2 Theta.

157. The pharmaceutical composition of claim 155, further comprising PVOH.

158. The pharmaceutical composition of claim 155, wherein said ribavirin is present in the amount of about 90.0% to about 99.5% by weight of said pharmaceutical composition.

159. The pharmaceutical composition of claim 155, wherein said ribavirin is present in the amount of about 95.0% to about 99.5% by weight of said pharmaceutical composition.

160. A powder ribavirin composition which achieves a determined Cmax of dissolved ribavirin in human bronchial epithelial lining fluid from 10 μM to 1.5 mM.

161. The composition of claim 160, wherein the determined Cmax is 10 μM to 1mM.

162. The composition of claim 160, wherein the determined Cmax is 10 μM to 500 μM.

163. The composition of claim 160, wherein the determined Cmax is 50 μM to 500 μM.

164. The composition of claim 160, wherein the determined Cmax is 50 μM to 100 μM.

165. The composition of claim 160, wherein the determined Cmax is 100 μM to 1 mM.
